(19) United States
(12) Patent Application Publication
(10) Pub. No.: US 2022/0363709 A1
(43) Pub. Date: Nov. 17, 2022

(54) ECTONUCLEOTIDASE INHIBITORS AND METHODS OF USE THEREOF

(71) Applicant: CALITHERA BIOSCIENCES, INC., South San Francisco, CA (US)

(72) Inventors: Lijing CHEN, Cupertino, CA (US); Roland Joseph BILLEDEAU, Santa Clara, CA (US); Jim LI, San Francisco (NY); Timothy Friend STANTON, Pacifica, CA (US)

(21) Appl. No.: 17/620,508

(22) PCT Filed: Jun. 18, 2020
(86) PCT No.: PCT/US2020/038395

§ 371 Date: Dec. 17, 2021

Related U.S. Application Data
(60) Provisional application No. 62/864,031, filed on Jun. 20, 2019.

(51) Int. Cl. C07H 19/167; A61K 31/7076; A61K 35/00

(52) U.S. Cl. CPC C07H 19/167; A61K 31/7076; A61K 35/00

(57) ABSTRACT

The invention relates to novel heterocyclic compounds and pharmaceutical preparations thereof. The invention further relates to methods of treating or preventing cancer using the novel heterocyclic compounds of the invention.

### RELATED APPLICATION

This application claims the benefit of priority to U.S. Provisional Patent Application No. 62/864,031, filed Jun. 20, 2019, which application is hereby incorporated by reference in its entirety.

### BACKGROUND

CD73, also referred to as 5′-nucleotidase (5′-NT) or ecto-5′-nucleotidase (Ecto 5′NTase), is a membrane-bound cell surface enzyme whose primary role is to catalyze the conversion of extracellular nucleotides (e.g., AMP) to their corresponding nucleosides (e.g., adenosine). CD73 is found in most tissues and expressed on lymphocytes, endothelial cells, and epithelial cells. It is also widely expressed in many tumor cell lines and, notably, is upregulated in cancerous tissues (Antonioli et al., Nat. Rev. Cancer, 13: 842-857, 2013).

In tandem with CD39 (ecto-ATPase), CD73 generates adenosine from ATP/AMP, which is often released from damaged or inflamed cells into the extracellular environment. Extracellular adenosine produced by CD73 interacts with G-protein coupled receptors on target cells. An important downstream effect of this signaling is increased immunosuppression via a number of pathways. For example, CD73 is a co-signaling molecule on T lymphocytes. Under normal circumstances, extracellular adenosine levels promote a self-limiting immune response that prevents excessive inflammation and tissue damage. For tumors, an advantage of abnormally increased CD73 is that the resulting increased CD73-catalyzed adenosine levels yield inhibition of anti-tumor immune system responses.

Even though CD73 plays a role in cancer immunosuppression, higher expression of CD73 is associated with a variety of stages of tumor progression, including tumor vascularization, invasiveness, and metastasis, and with shorter breast cancer patient survival time. Some of these observations result from CD73's enzyme-independent function as an adhesion molecule required for lymphocyte binding to the endothelium.

Overall, CD73 has become an important target for developing new cancer therapies, either as single agents or in combination with other cancer therapies. Indeed, combining CD73 monoclonal antibodies with antibodies for other chemotherapy targets enhances response and survival in animal cancer models (Allard et al., Clin. Cancer Res., 19:5626-35, 2013).

Many of the current cancer treatments and chemotherapeutic agents fail to successfully treat all patients or all symptoms in treated patients, and many of these therapies are associated with undesirable side effects. As certain cancers develop resistance to various chemotherapeutic agents, alternate cancer therapies are needed. Thus, there is a need for additional compounds and methods for treating cancer and other diseases.

### SUMMARY

Disclosed herein are compounds of Formula (I):

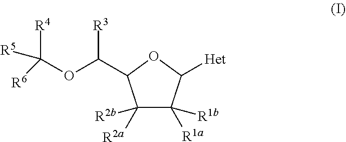

or a pharmaceutically acceptable salt and/or prodrug thereof, wherein

Het is heterocyclyl or heteroaryl;

R^{1a} is selected from H, halo, hydroxy, cyano, azido, amino, —O—C(O)—O—C₁₋₆alkyl, C₁₋₆acyloxy, and C₁₋₆alkoxy;

R^{1b} is selected from H and halo;

R^{2a} is selected from H, halo, hydroxy, cyano, azido, amino, C₁₋₆acyloxy, —O—C(O)—O—C₁₋₆alkyl, and C₁₋₆alkoxy;

R^{2b} is selected from H and halo;

R³ is selected from H and alkyl;

R⁴ is selected from aryl and heteroaryl;

R⁵ is selected from aralkyl and heteroaralkyl:

R⁶ is selected from —C(O)OR⁹, —C(O)NR¹³R¹⁴, —S(O)₂R¹⁰ and —P(O)(OR¹¹)(OR¹²);

R⁹ is independently selected from H, alkyl, cycloalkyl, cycloalkylalkyl, heterocyclyl, heterocyclylalkyl, aryl, aralkyl, heteroaryl, and heteroaralkyl;

R¹⁰ is independently selected from alkyl, alkenyl, alkynyl, amino, cycloalkyl, cycloalkylalkyl, heterocyclyl, heterocyclylalkyl, aryl, aralkyl, heteroaryl, and heteroaralkyl; and

R¹¹, R¹² and R¹⁴ are independently selected from H, alkyl, cycloalkyl, cycloalkylalkyl, heterocyclyl, heterocyclylalkyl, aryl, aralkyl, heteroaryl, heteroaralkyl; and

R¹³ is selected from H, hydroxy, alkyl, cycloalkyl, cycloalkylalkyl, heterocyclyl, heterocyclylalkyl, aryl, aralkyl, heteroaryl, heteroaralkyl;

provided that

if R⁴ is unsubstituted or substituted tetrazolyl, and

R⁶ is —C(O)OR⁹, then

R⁵ is not unsubstituted —CH₂-pyridyl, unsubstituted —CH₂-thienyl, —CH₂-thienyl substituted with a —C(O)OH group, unsubstituted benzyl, or benzyl substituted with a trifluoromethyl, trifluoromethoxy, methoxycarbonyl, —C(O)OH, benzyloxy, or phenyl group.

In certain embodiments, the present invention provides a pharmaceutical composition suitable for use in a subject in the treatment or prevention of cancer comprising an effective amount of any of the compounds described herein (e.g., a compound of the invention, such as a compound of Formula (I), or a pharmaceutically acceptable salt thereof, and one or more pharmaceutically acceptable excipients. In certain embodiments, the pharmaceutical preparations may be for use in treating or preventing a condition or disease as described herein.

Disclosed herein are methods of treating diseases and conditions that benefit from the inhibition of CD73, comprising administering to a subject in need thereof an effective amount of a compound as disclosed herein (e.g., a compound of Formula (I) or any of the embodiments thereof disclosed herein). In certain embodiments, the human subject is in need of such treatment. These diseases include, but are not limited to cancers, such as lung cancer, kidney cancer, skin cancer, breast cancer, and ovarian cancer. Other diseases and conditions that can be treated using the methods described herein include, but are not limited to, neurological, neurodegenerative and CNS disorders and diseases such as depression and Parkinson's disease, cerebral and cardiac ischemic diseases, sleep disorders, fibrosis, immune and inflammatory disorders.

Provided herein are combination therapies of compounds of formula (I) with monoclonal antibodies and other chemotherapeutic agents that can enhance the therapeutic benefit beyond the ability of the adjuvant therapy alone.

### DETAILED DESCRIPTION

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In some embodiments, chemical structures are disclosed with a corresponding chemical name. In case of conflict, the chemical structure controls the meaning, rather than the name.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not substantially changed by the presence of more than that which is recited, but excludes prior art embodiments.

Unless specifically stated or obvious from context, as used herein, the term “or” is understood to be inclusive. Unless specifically stated or obvious from context otherwise, as used herein, the terms “a”, “an”, and “the” are understood to be singular or plural.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

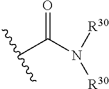

wherein each R³⁰ independently represents a hydrogen or hydrocarbyl group, or two R³⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

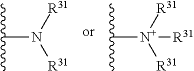

wherein each R³¹ independently represents a hydrogen or a hydrocarbyl group, or two R³¹ are taken together with the N atom to which they are attached complete a heterocycle having from 4 to 8 atoms in the ring structure. The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

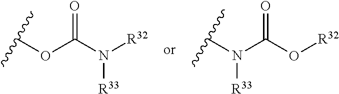

wherein R³² and R³³ independently represent hydrogen or a hydrocarbyl group, such as an alkyl group, or R³² and R³³ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R³⁴, wherein R³⁴ represents a hydrocarbyl group.

The term “carboxy”, as used herein, refers to a group represented by the formula —CO₂H.

The term “ester”, as used herein, refers to a group —C(O)OR³⁵ wherein R³⁵ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

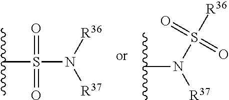

wherein R³⁶ and R³⁷ independently represent hydrogen or hydrocarbyl, such as alkyl, or R³⁶ and R³⁷ taken together with the intervening atom(s) complete a heterocycle having from 4 to 8 atoms in the ring structure.

The term “sulfoxide” is art-recognized and refers to the group —S(O)—R³⁸, wherein R³⁸ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R³⁹, wherein R³⁹ represents a hydrocarbyl.

The term “thioalkyl”, as used herein, refers to an alkyl group substituted with a thiol group.

The term “thioester”, as used herein, refers to a group —C(O)SR⁴⁰ or —SC(O)R⁴⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

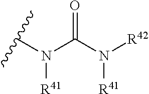

wherein R⁴¹ and R⁴² independently represent hydrogen or a hydrocarbyl, such as alkyl, or either occurrence of R⁴¹ taken together with R⁴² and the intervening atom(s) complete a heterocycle having from 4 to 8 atoms in the ring structure.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

In certain embodiments, compounds of the invention may be racemic. In certain embodiments, compounds of the invention may be enriched in one enantiomer. For example, a compound of the invention may have greater than about 30% ee, about 40% ee, about 50% cc, about 60% cc, about 70% cc, about 80% cc, about 90% cc, or even about 95% or greater cc. In certain embodiments, compounds of the invention may have more than one stereocenter. In certain such embodiments, compounds of the invention may be enriched in one or more diastereomer. For example, a compound of the invention may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., of Formula (I)). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of Formula (I)). A diastereomerically enriched mixture may comprise, for example, at least about 60 mol percent of one diastereomer, or more preferably at least about 75, about 90, about 95, or even about 99 mol percent.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of Formula (I)). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present invention. In certain embodiments, some or all of the compounds of Formula (I) in a formulation represented above can be replaced with the corresponding suitable prodrug, e.g., wherein a hydroxyl in the parent compound is presented as an ester or a carbonate or carboxylic acid.

An “effective amount”, as used herein, refers to an amount that is sufficient to achieve a desired biological effect. A “therapeutically effective amount”, as used herein, refers to an amount that is sufficient to achieve a desired therapeutic effect. For example, a therapeutically effective amount can refer to an amount that is sufficient to improve at least one sign or symptom of cancer.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

In some embodiments, the invention provides a compound of formula (I):

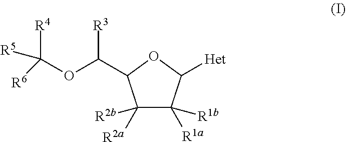

or a pharmaceutically acceptable salt and/or prodrug thereof, wherein

Het is heterocyclyl or heteroaryl;

R^{1a} is selected from H, halo, hydroxy, cyano, azido, amino, —O—C(O)—O—C₁₋₆alkyl, C₁₋₆acyloxy, and C₁₋₆alkoxy:

R^{1b} is selected from H and halo;

R^{2a} is selected from H, halo, hydroxy, cyano, azido, amino, C₁₋₆acyloxy, —O—C(O)—O—C₁₋₆alkyl, and C₁₋₆alkoxy;

R^{2b} is selected from H and halo;

R³ is selected from H and alkyl;

R⁴ is selected from aryl and heteroaryl;

R⁵ is selected from aralkyl and heteroaralkyl:

R⁶ is selected from —C(O)OR⁹, —C(O)NR¹³R¹⁴, —S(O)₂R¹⁰ and —P(O)(OR¹¹)(OR¹²);

R⁹ is independently selected from H, alkyl, cycloalkyl, cycloalkylalkyl, heterocyclyl, heterocyclylalkyl, aryl, aralkyl, heteroaryl, and heteroaralkyl;

R¹⁰ is independently selected from alkyl, alkenyl, alkynyl, amino, cycloalkyl, cycloalkylalkyl, heterocyclyl, heterocyclylalkyl, aryl, aralkyl, heteroaryl, and heteroaralkyl; and

R¹¹, R¹² and R¹⁴ are independently selected from H, alkyl, cycloalkyl, cycloalkylalkyl, heterocyclyl, heterocyclylalkyl, aryl, aralkyl, heteroaryl, heteroaralkyl; and

R¹³ is selected from H, hydroxy, alkyl, cycloalkyl, cycloalkylalkyl, heterocyclyl, heterocyclylalkyl, aryl, aralkyl, heteroaryl, heteroaralkyl;

provided that

if R⁴ is unsubstituted or substituted tetrazolyl, and

R⁶ is —C(O)OR⁹, then

R⁵ is not unsubstituted —CH₂-pyridyl, unsubstituted —CH₂-thienyl, —CH₂-thienyl substituted with a —C(O)OH group, unsubstituted benzyl, or benzyl substituted with a trifluoromethyl, trifluoromethoxy, methoxycarbonyl, —C(O)OH, benzyloxy, or phenyl group.

In certain embodiments, R^{1a} is H or hydroxy. In certain embodiments, R^{1b} is H. In other embodiments, R^{2a} is H or hydroxy. In some embodiments, R^{2b} is H. In preferred embodiments, R^{1a} is hydroxy, R^{1b} is H, R^{2a} is hydroxy, and R^{2b} is H. In some embodiments, R^{1a} is H and R^{1b} is halo, preferably F.

In certain preferred embodiments, R³ is H.

In certain embodiments, the compound of Formula (I) has the following structure:

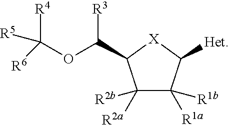

In certain such embodiments, R^{1a} is in the α-configuration. For example, the compound of Formula (I) may have the structure (IA):

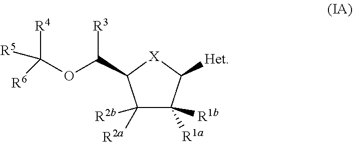

In alternative embodiments, R^{1a} is in the β-configuration. In some such embodiments, the compound of Formula (I) may have the structure (IB):

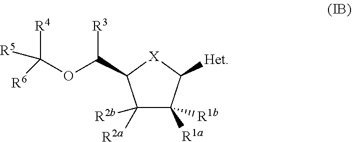

In further embodiments of compounds of Formula (I), e.g., as described above, R^{2a} is in the α-configuration. For example, the compound of Formula (I) may have the structure (IC):

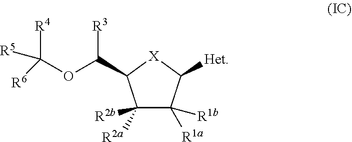

In alternative embodiments, R^{2a} is in the β-configuration. In some such embodiments, the compound of Formula (I) may have the structure (ID):

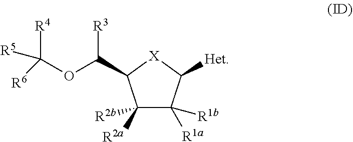

In certain preferred embodiments, the compound of Formula (I) has the structure (IE):

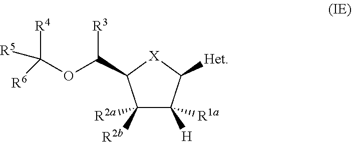

In certain embodiments, R⁴ is selected from aryl and heteroaryl, e.g., heteroaryl. In certain preferred embodiments, R⁴ is heteroaryl selected from thiazolyl, pyrazolyl, triazolyl, oxazolyl, and thienyl.

In certain embodiments, R⁵ is selected from aralkyl and heteroaralkyl. In certain such embodiments, each aralkyl and heteroaralkyl at R⁵ is unsubstituted or substituted with one or more substituents selected from carboxy, heteroaryl and aryl, preferably heteroaryl or aryl.

In certain preferred embodiments, R⁵ is aralkyl substituted on the aryl ring (e.g., a benzyl substituted at a para-position of the phenyl ring) with a second aryl or heteroaryl ring (preferably a phenyl ring) unsubstituted or substituted with one or more substituents, e.g., selected from hydroxyl, cyano, alkyl, alkoxy, amido, carboxy, alkoxycarbonyl, heterocyclyl, heteroaryl, and sulfonamido.

In certain preferred embodiments, R⁵ is benzyl substituted on the phenyl ring (e.g., at the 4-position) with

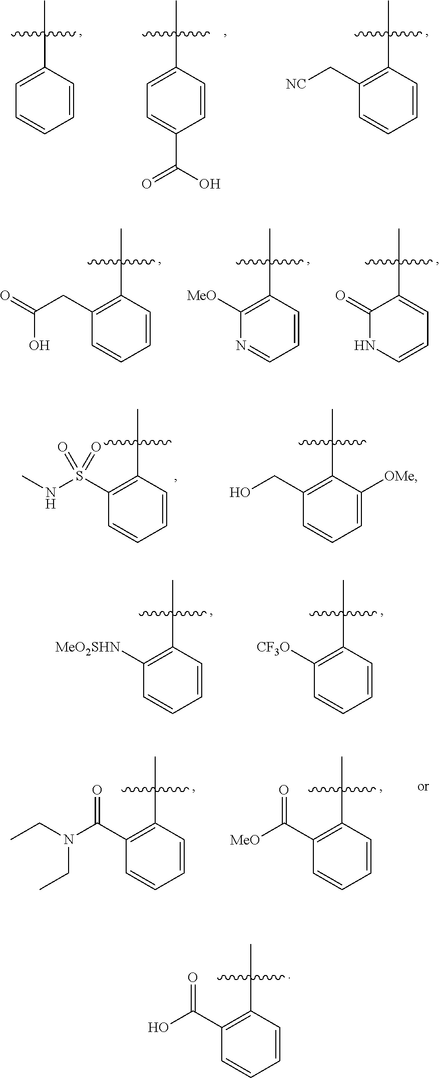

In some embodiments, R⁶ is —C(O)OR⁹ and R⁹ is H or alkyl, e.g., H or C₁₋₆alkyl.

In certain embodiments,

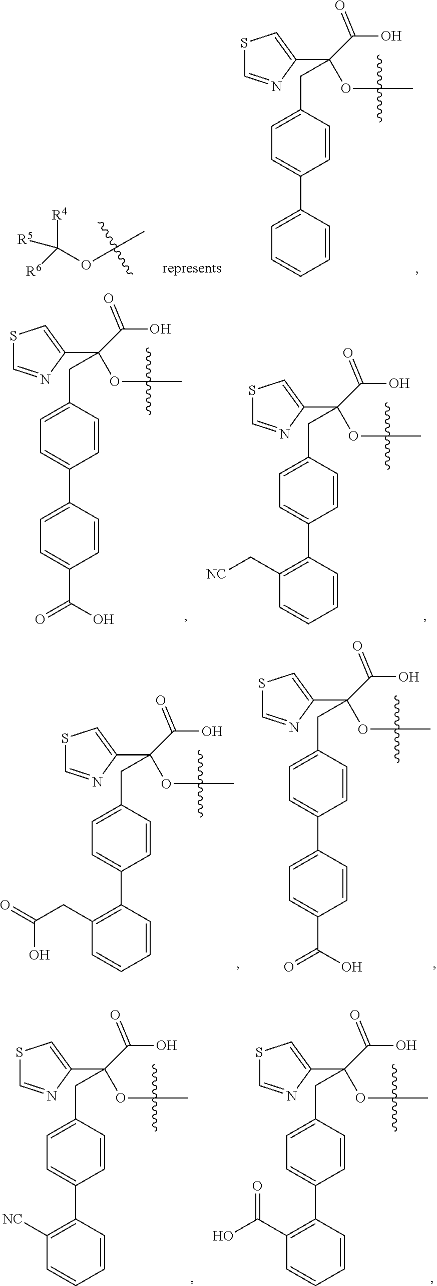

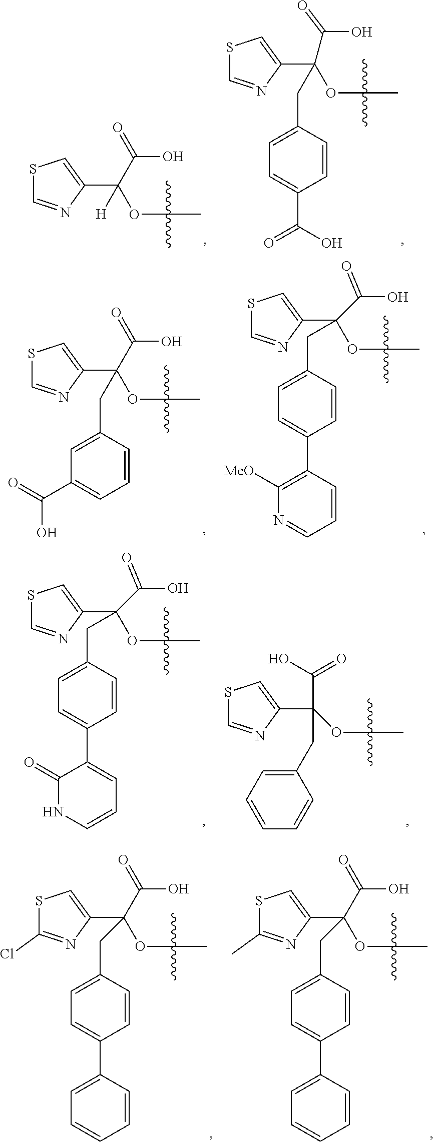

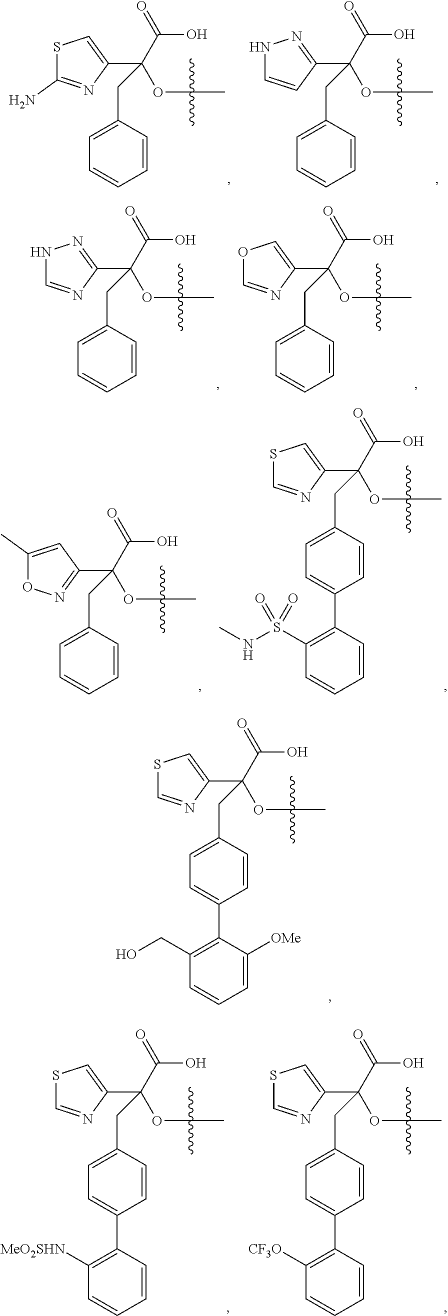

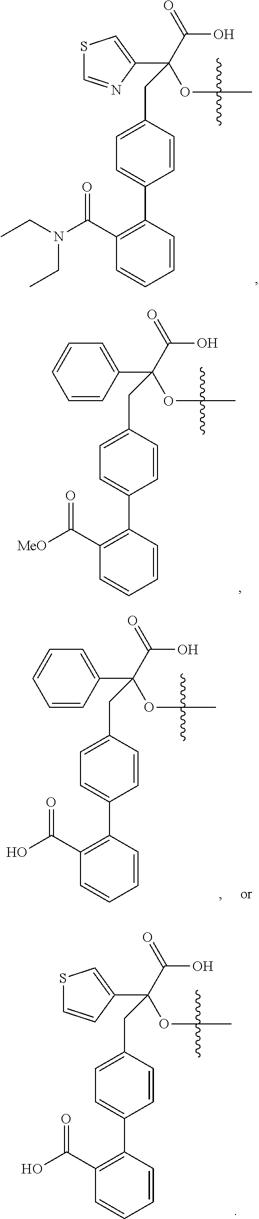

In certain embodiments, Het is selected from a 6- to 10-membered aryl, a 5- to 8-membered heterocyclyl, a 5- to 8-membered monocyclic or 5- to 10-membered bicyclic heteroaryl, and may be unsubstituted or substituted with one or more substituents selected from halo, alkoxy, and amino. In some embodiments, the Het substituents are selected from halo and amino. In certain embodiments, Het is a nitrogen-containing heterocyclyl or heteroaryl, preferably attached to the core ring via a nitrogen atom of the heterocyclyl or heteroaryl ring.

In other embodiments, Het is

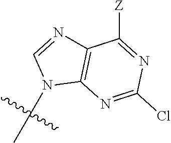

wherein

Z is OR⁷ or NR⁷R⁸

R⁷ is selected from H, alkyl, aralkyl, heteroaralkyl, cycloalkyl, and heterocyclyl; and
R⁸ is H or alkyl.

In some embodiments, R⁷ is alkyl and R⁸ is H.

### Methods of Use

Provided herein are methods of inhibiting CD73 in a cell, comprising contacting the cell with a compound of the invention, such as a compound of formula (I), or a pharmaceutically acceptable salt thereof. In certain embodiments, contacting the cell occurs in a subject in need thereof, thereby treating a disease or disorder mediated by adenosine.

Also, disclosed herein are methods of treating a disease or a disorder mediated by adenosine comprising administering a compound the invention, such as a compound of of Formula (I), or a pharmaceutically acceptable salt thereof. In some embodiments, disclosed herein are methods of treating cancer comprising administering a compound the invention, such as a compound of Formula (I), or a pharmaceutically acceptable salt thereof.

Adenosine acts on a variety of immune cells to induce immunosuppression, and the immunosuppressive effects of ectonucleotidases that enhance adenosine levels are also associated with enhanced infections of mammalian cells by parasites, fungi, bacteria, and viruses. Apart from immunosuppressive effects, adenosine also has a role in modulating the cardiovascular system (as a vasodilator and cardiac depressor), the central nervous system (CNS) (inducing sedative, anxiolytic and antiepileptic effects), the respiratory system (inducing bronchoconstriction), the kidney (having biphasic action; inducing vasoconstriction at low concentrations and vasodilation at high doses), fat cells (inhibiting lipolysis), and platelets (as an anti-aggregant). Furthermore, adenosine also promotes fibrosis (excess matrix production) in a variety of tissues. Therefore, improved treatments targeting CD73 would provide therapies for treating a wide range of conditions in addition to cancer, including cerebral and cardiac ischemic disease, fibrosis, immune and inflammatory disorders (e.g., inflammatory gut motility disorder), neurological, neurodegenerative and CNS disorders and diseases (e.g., depression, Parkinson's disease), and sleep disorders.

In some embodiments, the disease or the disorder mediated by adenosine is selected from cerebral ischemic disease, cancer, cardiac ischemic disease, depression, fibrosis, an immune disorder, an inflammatory disorder (e.g., inflammatory gut motility disorder), neurological disorder or disease, neurodegenerative disorder or disease (e.g., Parkinson's disease). CNS disorders and diseases, and sleep disorders.

The methods described herein are useful for the treatment of a wide variety of cancers, including bladder cancer, bone cancer, brain cancer (including glioblastoma), breast cancer, cardiac cancer, cervical cancer, colon cancer, colorectal cancer, esophageal cancer, fibrosarcoma, gastric cancer, gastrointestinal cancer, head & neck cancer, Kaposi's sarcoma, kidney cancer (including renal cell adenocarcinoma), leukemia, liver cancer, lung cancer (including non-small cell lung cancer, small cell lung cancer, and mucoepidermoid pulmonary carcinoma), lymphoma, melanoma, myeloma, ovarian cancer (including ovarian adenocarcinoma), pancreatic cancer, penile cancer, prostate cancer, testicular germcell cancer, thymoma and thymic carcinoma.

In some embodiments, the subject has a cancer selected from breast cancer, brain cancer, colon cancer, fibrosarcoma, kidney cancer, lung cancer, melanoma, ovarian cancer, and prostate cancer. In certain embodiments, the subject has a cancer selected from breast cancer, colon cancer, fibrosarcoma, melanoma, ovarian cancer, and prostate cancer. In other embodiments, the subject has a cancer selected from brain cancer, breast cancer, kidney cancer, lung cancer, melanoma, and ovarian cancer. In some embodiments, the subject has head and neck squamous cell carcinoma, ovarian cancer, breast cancer or esophageal cancer. In other embodiments, the subject has pancreatic cancer, esophageal cancer, stomach cancer, head and neck cancer, colon cancer, lung cancer or kidney cancer. In yet other embodiments, the subject has breast cancer. In some embodiments, the breast cancer is breast adenocarcinoma. In certain embodiments, the breast cancer is triple-negative breast cancer.

In certain embodiments, the methods for treating or preventing cancer can be demonstrated by one or more responses such as increased apoptosis, inhibition of tumor growth, reduction of tumor metastasis, inhibition of tumor metastasis, reduction of microvessel density, decreased neovascularization, inhibition of tumor migration, tumor regression, and increased survival of the subject.

In certain embodiments, the disease or the disorder mediated by adenosine is a disease or disorder mediated by CD73 activity. In some embodiments, the compounds of the invention, such as compounds of Formula (I), are useful as inhibitors of CD73.

In some embodiments, the methods described herein treat or prevent cardiovascular disease using inhibitors of CD73. Mutant genes encoding CD73 lead to extensive calcification of lower-extremity arteries and small joint capsules, which is associated with increased risk of cardiovascular disease (Hilaire et al., N. Engl. J. Med., 364(5): 432-442, 2011).

In some embodiments, the methods disclosed herein treat or prevent cancer using inhibitors of CD73. A CD73 small interfering RNA and anti-CD73 monoclonal antibodies showed a significant effect in treating or preventing cancer (Antonioli et al., Nat. Rev. Cancer, 13: 842-857, 2013). A tight correlation exists between CD73 expression and the ability of cancer cells to migrate, invade, and adhere to the extracellular matrix (ECM) (Antonioli 2013; Antonioli et al., Trends Cancer, 2(2): 95-109, 2016).

In some embodiments, the treatment or prevention of cancer by inhibitors of CD73 can be demonstrated by one or more responses selected from activation, clonal expansion, and homing of tumor-specific T cells (Antonioli 2016). In other embodiments, the methods disclosed herein increase the number of effector T lymphocytes (e.g., cytolytic effector T lymphocytes).

### Combination Treatments

In some embodiments, the method of treating or preventing cancer may comprise administering a CD39 inhibitor conjointly with one or more other chemotherapeutic agent(s). In one embodiment, the CD73 inhibitor is a compound of the invention, such as a compound of Formula (I). Other chemotherapeutic agents can include CD73-specific monoclonal antibodies which enhance the effects of other antibodies and therapies because of increased overall immune system activity (lower T-regulatory function and higher T-effector function, etc.) (Antonioli 2016).

In certain embodiments, the method of treating or preventing cancer may comprise administering a compound of the invention conjointly with one or more other chemotherapeutic agent(s).

Chemotherapeutic agents that may be conjointly administered with compounds of the invention include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, I-amino-4-[2-anthracenylamino]-9,10-dioxo-9,10-dihydroanthracene-2-sulfonate, ABT-263, afatinib dimaleate, axitinib, aminoglutethimide, amsacrine, anastrozole, APCP, asparaginase, AZD5363, Bacillus Calmette-Guérin vaccine (bcg), bicalutamide, bleomycin, bortezomib, β-methylene-ADP (AOPCP), buserelin, busulfan, cabazitaxel, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, N-(4-sulfamoylphenylcarbamothioyl) pivalamide, NF279, NF449, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, PPADS, procarbazine, quercetin, raltitrexed, ramucirumab, reactive blue 2, rituximab, rolofylline, romidepsin, rucaparib, selumetinib, sirolimus, sodium 2,4-dinitrobenzenesulfonate, sorafenib, streptozocin, sunitinib, suramin, talazoparib, tamoxifen, temozolomide, temsirolimus, teniposide, testosterone, thalidomide, thioguanine, thiotepa, titanocene dichloride, tonapofylline, topotecan, trametinib, trastuzumab, tretinoin, veliparib, vinblastine, vincristine, vindesine, vinorelbine, and vorinostat (SAHA). In other embodiments, chemotherapeutic agents that may be conjointly administered with compounds of the invention include: ABT-263, dexamethasone, 5-fluorouracil, PF-04691502, romidepsin, and vorinostat (SAHA). In other embodiments, chemotherapeutic agents that may be conjointly administered with compounds of the invention include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, APCP, β-methylene-ADP (AOPCP), capecitabine, cladribine, cytarabine, fludarabine, doxorubicin, gemcitabine, N-(4-sulfamoylphenylcarbamothioyl) pivalamide, NF279, NF449, PPADS, quercetin, reactive blue 2, rolofylline sodium 2,4-dinitrobenzenesulfonate, sumarin, and tonapofylline.

Many combination therapies have been developed for the treatment of cancer. In certain embodiments, compounds of the invention (e.g., compounds of Formula (I)) may be conjointly administered with a combination therapy. Examples of combination therapies with which compounds of the invention may be conjointly administered are included in Table 1.

TABLE 1
Exemplary combinatorial therapies for the treatment of cancer

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or |
| | without/Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic | Chlorambucil, Prednisone |
| lymphocytic leukemia) | |
| CP (Ovarian Cancer) | Cyclophosphamide, Cisplatin |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple | Melphalan, Prednisone |
| myeloma) | |
| MP (prostate cancer) | Mitoxantrone, Prednisone |
| MTX76-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PCV | Lomustine, Procarbazine, Vincristine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, |
| | Procarbazine, Hydroxyurea, Cisplatin, Cytarabine, |
| | Dacarbazine |

In some embodiments, the chemotherapeutic agents that may be conjointly administered with compounds of the invention, such as a compound of Formula (I), include a CD39 inhibitor. CD39 or ecto-nucleoside triphosphate diphosphohydrolase 1 (E-NTPDase 1 or ENTPD 1) is a membrane-bound enzyme that catalyzes the conversion of extracellular adenosine triphosphate (ATP) and/or ADP (adenosine driphosphate) to adenosine monophosphate (AMP). In one embodiment, the CD39 inhibitor is polyoxometalate-1 (POM-1).

In other embodiments, the chemotherapeutic agents that may be conjointly administered with compounds of the invention, such as a compound of Formula (I), include known CD73 inhibitors. In some embodiments, the CD73 inhibitor is an anthraquinone derivative (Baqi et al., J. Med. Chem., 53(5): 2076-2086, 2010, herein incorporated by reference). In other embodiments, the CD73 inhibitor is an sulfonic acid derivative (Raza et al., Med. Chem., 8: 1133-1139, 2012, herein incorporated by reference). In yet other embodiments, the CD73 inhibitor is selected from 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, I-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, sodium 2,4-dinitrobenzenesulfonate, N-(4-sulfamoylphenylcarbamothioyl) pivalamide, APCP, β-methylene-ADP (AOPCP), PPADS, NF279, NF449, quercetin, reactive blue 2, and sumarin (Baqi 2010; Raza 2012).

In certain embodiments, the combination of a compound of the invention, such as a compound of Formula (I), with a second CD73 inhibitor or a CD39 inhibitor may have a synergistic effect in the treatment of cancer and other diseases or disorders mediated by adenosine. Without wishing to be bound by any theory, this synergy may be observed because CD39 and CD73 are often on different cell types. The hypoxic tumor microenvironment also induces greater levels of CD39 and CD73.

In some embodiments, the chemotherapeutic agents that may be conjointly administered with compounds of the invention, such as a compound of Formula (I), include an adenosine receptor inhibitor. In other embodiments, the adenosine receptor inhibitor is selected from roofylline, tonapofylline, ATL-444, istradefylline, MSX-3, preladenant, SCH-58,261, SCH-412,348, SCH-442,416, ST-1535, VER-6623, VER-6947, VER-7835, vipadenant, and ZM-241,385. In some embodiments, the adenosine receptor inhibitor targets the A_{2A} receptor as this subtype is predominantly expressed in most immune cells.

In other embodiments, the chemotherapeutic agents that may be conjointly administered with compounds of the invention, such as a compound of Formula (I), include a nucleoside-based drug. In certain embodiments, the nucleoside-based drug is selected from gemcitabine, capecitabine, cytarabine, fludarabine and cladribine.

In further embodiments, the combination therapy comprises a compound of the invention, such as a compound of Formula (I), conjointly administered with an anthracycline. In other embodiments, the combination therapy comprises a compound of the invention, such as a compound of Formula (I), conjointly administered with doxorubicin. Combination treatment with an anti-CD73 antibody and doxorubicin has demonstrated a significant chemotherapeutic effect (Young et al., Cancer Discov., 4(8): 1-10, 2014, herein incorporated by reference).

In certain embodiments, the combination therapy comprises a compound of the invention, such as a compound of Formula (I), conjointly administered with an A_{2A} receptor inhibitor and an anthracycline. In some embodiments, the anthracycline is doxorubicin. Combination treatment with an anti-CD73 antibody, an A_{2A} receptor inhibitor, and doxorubicin has demonstrated an increased chemotherapeutic effect (Antonioli 2013).

In certain embodiments, the conjoint therapies of the invention comprise conjoint administration with other types of chemotherapeutic agents, such as immuno-oncology agents. Cancer cells often have specific cell surface antigens that can be recognized by the immune system. Thus, immuno-oncology agents, such as monoclonal antibodies, can selectively bind to cancer cell antigens and effect cell death. Other immuno-oncology agents can suppress tumor-mediated inhibition of the native immune response or otherwise activate the immune response and thus facilitate recognition of the tumor by the immune system. Exemplary antibody immuno-oncology agents, include, but are not limited to, abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, and tremelimumab. In some embodiments, the antibody immune-oncology agents are selected from anti-CD73 monoclonal antibody (mAb), anti-CD39 mAb, anti-PD-1 mAb, and anti-CTLA4 mAb. Thus, in some embodiments, the methods of the invention comprise conjoint administration of one or more immuno-oncology agents, such as the agents mentioned above.

In some embodiments, the combination therapy comprises a compound of the invention, such as a compound of Formula (I), conjointly administered with anti-PD-1 therapy and anti-CTLA4 therapy. Combination treatment with an anti-CD73 monoclonal antibody (mAb), anti-PD-1 mAb, and anti-CTLA4 mAb showed a significant chemotherapeutic effect (Young 2014; Antonioli 2013).

In some embodiments, the combination therapy comprises conjoint administration of a compound of the invention, such as a compound of Formula (I), with anti-PD-1 therapy. In certain embodiments, the combination therapy comprises conjoint administration of a compound of the invention, such as a compound of Formula (I), with oxaliplatin. In other embodiments, the combination therapy comprises conjoint administration of a compound of the invention, such as a compound of Formula (I), with doxorubicin.

In certain embodiments, a compound of the invention may be conjointly administered with non-chemical methods of cancer treatment. In certain embodiments, a compound of the invention may be conjointly administered with radiation therapy. In certain embodiments, a compound of the invention may be conjointly administered with surgery, with thermoablation, with focused ultrasound therapy, with cryotherapy, or with any combination of these.

In certain embodiments, compounds of the invention may be conjointly administered with one or more other compounds of the invention. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound of the invention provides a synergistic effect. In certain embodiments, conjointly administering one or more additional chemotherapeutic agents provides an additive effect.

### Pharmaceutical Compositions

In certain embodiments, the present invention provides a pharmaceutical preparation suitable for use in a human patient, comprising any of the compounds shown above (e.g., a compound of the invention, such as a compound of formula (I), and one or more pharmaceutically acceptable excipients. In certain embodiments, the pharmaceutical preparations may be for use in treating or preventing a condition or disease as described herein. Any of the disclosed compounds may be used in the manufacture of medicaments for the treatment of any diseases or conditions disclosed herein.

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, the dosing follows a 3+3 design. The traditional 3+3 design requires no modeling of the dose-toxicity curve beyond the classical assumption for cytotoxic drugs that toxicity increases with dose. This rule-based design proceeds with cohorts of three patients; the first cohort is treated at a starting dose that is considered to be safe based on extrapolation from animal toxicological data, and the subsequent cohorts are treated at increasing dose levels that have been fixed in advance. In some embodiments, the three doses of a compound of formula (I) range from about 100 mg to about 1000 mg orally, such as about 200 mg to about 800 mg, such as about 400 mg to about 700 mg, such as about 100 mg to about 400 mg, such as about 500 mg to about 1000 mg, and further such as about 500 mg to about 600 mg. Dosing can be three times a day when taken with without food, or twice a day when taken with food. In certain embodiments, the three doses of a compound of formula (I) range from about 400 mg to about 800 mg, such as about 400 mg to about 700 mg, such as about 500 mg to about 800 mg, and further such as about 500 mg to about 600 mg twice a day. In certain preferred embodiments, a dose of greater than about 600 mg is dosed twice a day.

If none of the three patients in a cohort experiences a dose-limiting toxicity, another three patients will be treated at the next higher dose level. However, if one of the first three patients experiences a dose-limiting toxicity, three more patients will be treated at the same dose level. The dose escalation continues until at least two patients among a cohort of three to six patients experience dose-limiting toxicities (i.e., ≥about 33% of patients with a dose-limiting toxicity at that dose level). The recommended dose for phase II trials is conventionally defined as the dose level just below this toxic dose level.

In certain embodiments, the dosing schedule can be about 40 mg/m² to about 100 mg/m², such as about 50 mg/m² to about 80 mg/m², and further such as about 70 mg/m² to about 90 mg/m² by IV for 3 weeks of a 4 week cycle.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially.

In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of formula I or Ia) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The invention now being generally described, it will be more readily understood by reference to the following examples which are included merely for purposes of illustration of certain aspects and embodiments of the present invention, and are not intended to limit the invention.

### General Synthetic Procedures

Compound numbers 1-50 as used in the general synthesis section below refer only to genus structures in this section and do not apply to compounds disclosed elsewhere in this application. Compounds disclosed herein can be made by methods depicted in the reaction schemes below.

The starting materials and reagents used in preparing these compounds are either available from commercial supplier such as Aldrich Chemical Co., Bachem, etc., or can be made by methods well known in the art. The schemes are merely illustrative of some methods by which the compounds disclosed herein can be synthesized and various modifications to these schemes can be made and will be suggested to POSITA having referred to this disclosure. The starting materials and the intermediates and the final products of the reaction may be isolated and purified if desired using conventional techniques, including but not limited to filtration, distillation, crystallization, chromatography, and the like and may be characterized using conventional means, including physical constants and spectral data.

Unless specified otherwise, the reactions described herein take place at atmospheric pressure over a temperature range from about −78° C. to about 150° C.

### General Scheme

Compounds of Formula (I) having the structure:

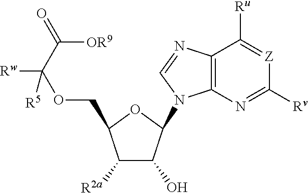

where Z, R^{u}, R^{v}, R^{2a}, R⁵, and R⁹ are analogous to variables Z, R^{2a}, R⁵, and R⁹ as defined in the Summary, can be synthesized as illustrated and described in Scheme 1 below.

Converting the acetate ester A-1 where R^{w} is either an aryl or heteroaryl group and R⁹ is an alkyl group, to the required diazo intermediate A-3 by the treatment of diazotization reagent such as 4-acetamidobenezensulfonyl azide (A-2) in the presence of a base such as DBU, TEA or Cs₂CO₃ in a solvent such as MeCN, or THF. The primary alcohol A-4, where R=^{2a} is H or OH, R⁹ is an alkyl group, P is a protecting group such as t-Boc, Ac, or TBS, and R^{v} and R^{u} are common substitutions such as H, alkyl, aryl, amino, alkoxy ether and thioether, is prepared according to the reported procedures (WO2018119284 and WO2018049145). Coupling of the resulting diazo intermediate A-3 with primary alcohol A-4 to provide A-5 via an insertion reaction catalyzed by a metal catalyst such as Rh₂(OAc)₄ in a solvent such as toluene, dichloromethane and dichloroethane. Alkylation of A-5 with an electrophile A-6 such as alkyl halide, triflate, tosylate or mesylate in the presence of a base such as Cs₂CO₃, K₂CO₃, LiHMDS, DBU or NaH, to provide A-7. Removal of the protecting groups in A-7 by TFA for P is a t-Boc group to give intermediate A-8. The ester group in A-8 is finally removed by a base such as LiOH, NaOH, KOH and NH₃ in an aqueous media to provide the desired product in formula (I).

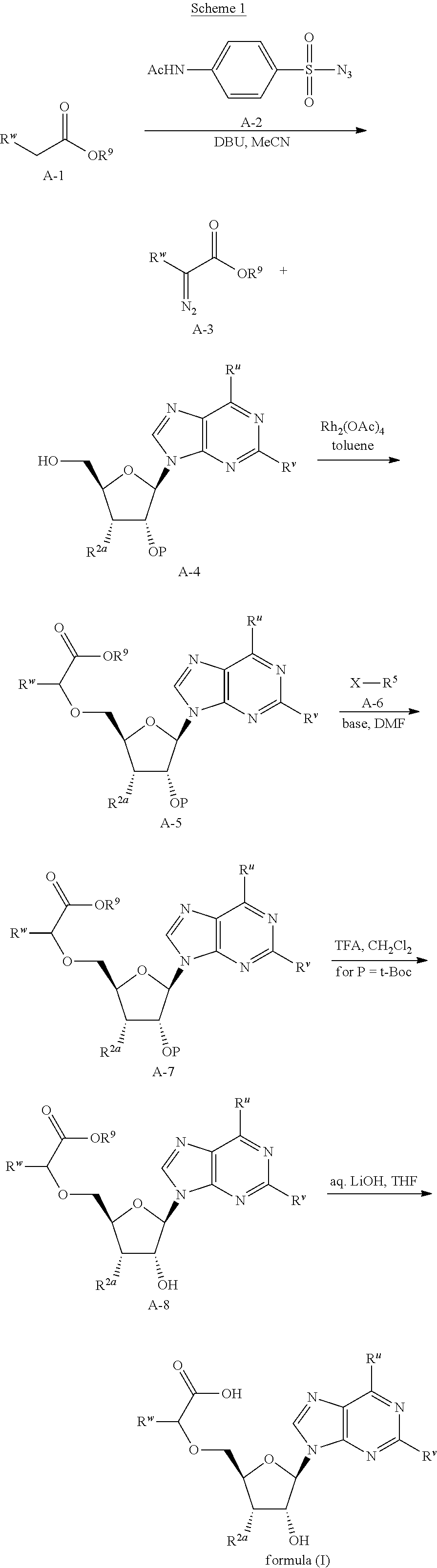

Those having skill in the art will recognize that the starting materials and reaction conditions may be varied, the sequence of the reactions altered, and additional steps employed to produce compounds encompassed by the present invention, as demonstrated by the following examples. In some cases, protection of certain reactive functionalities may be necessary to achieve some of the above transformations. In general, the need for such protecting groups as well as the conditions necessary to attach and remove such groups will be apparent to experienced organic chemists. The disclosures of all articles and references mentioned in this application, including patents, are incorporated herein by reference.

The preparation of the compounds of the present invention is illustrated further by the following examples, which are not to be construed as limiting the invention in scope or spirit to the specific procedures and compounds described in them.

### SYNTHETIC EXAMPLES

### Example 1

### Synthesis of 3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(thiazol-4-yl)propanoic acid

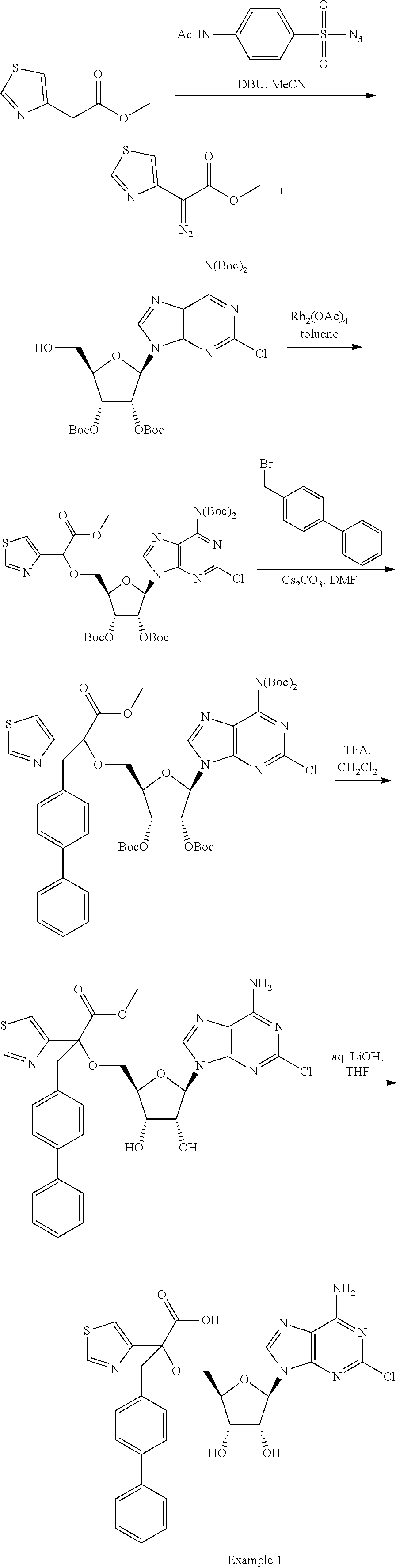

### Step 1:

To a solution of methyl 2-(thiazol-4-yl)acetate (1.84 g, 11.7 mmole) in CH₃CN (15 mL) at 0° C. was added DBU (2.62 ml, 17.6 mmole) and 4-acetamidibenzene sulfonylazide (3.4 g, 14.1 mmole) in CH₃CN (10 mL). The reaction mixture was stirred at 25° C. for 1.5 h before it was concentrated under reduced pressure to dryness. The resulting crude was purified by silica gel column chromatography (0-40% EtOAc in hexanes) to provide methyl 2-diazo-2-(thiazol-4-yl)acetate (2.0 g).

### Step 2:

To a solution of tert-butyl (9-((2R,3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate (1.0 g, 1.42 mmol) in toluene (10 mL) was methyl 2-diazo-2-(thiazol-4-yl)acetate (365 mg, 1.85 mmol) and Rh₂(OAc)₄ (63 mg, 0.14 mmol) under argon atmosphere. The resulting mixture was stirred at 70° C. for 1.5 h before it was allowed to cool to room temperature. The organic volatile was removed under reduced pressure. The resulting crude was purified by silica gel column chromatography (0-40% EtOAc in hexanes) to provide a diastereomeric mixture (ca. 1:1) of methyl 2-(((2R,3R,4R,5R)-5-(6-N,N′-(bis-(tert-butoxycarbonyl)amino)-2-chloro-9H-purin-9-yl)-3,4-bis((tert-butoxycarbonyl)oxy)tetra-hydrofuran-2-yl)methoxy)-2-(thiazol-4-yl)acetate.

### Step 3:

To a solution of a diastereomeric mixture (ca. 1:1) of ethyl 2-(((2R,3R,4R,5R)-5-(6-N,N′-(bis-(tert-butoxycarbonyl)amino)-2-chloro-9H-purin-9-yl)-3,4-bis((tert-butoxy-carbonyl)oxy)tetrahydrofuran-2-yl)methoxy)-2-(thiazol-4-yl)acetate (458 mg, 0.526 mmol) in DMF (2 mL) at 25° C. was added Cs₂CO₃ (145 mg, 0.446 mmol). The reaction mixture was stirred for 30 min and followed by addition of 4-(bromomethyl)-1,1′-biphenyl (260 mg, 1.051 mmol). The reaction mixture was stirred for overnight before it was diluted with H₂O (20 mL) and extracted with EtOAc (3×30 mL). The combined organic layer was washed further with H₂O (2×40 mL), brine, dried over Na₂SO₄ and concentrated. The resulting crude was purified by flash silica gel column chromatography (0-50% EtOAc in hexanes) to provide a diastereomeric mixture (ca. 1:1) of methyl 3-([1,1′-biphenyl]-4-yl)-2-(((2R,3R,4R,5R)-5-(6-N,N′-(bis-(tert-butoxycarbonyl)-amino)-2-chloro-9H-purin-9-yl)-3,4-bis((tert-butoxycarbonyl)oxy)tetrahydrofuran-2-yl)methoxy)-2-(thiazol-4-yl)propanoate.

### Step 4:

To a solution of a diastereomeric mixture (ca. 1:1) of methyl 3-([1,1′-biphenyl]-4-yl)-2-(((2R,3R,4R,5R)-5-(6-N,N′-(bis-(tert-butoxycarbonyl)amino)-2-chloro-9H-purin-9-yl)-3,4-bis((tert-butoxycarbonyl)oxy)tetra-hydrofuran-2-yl)methoxy)-2-(thiazol-4-yl)propanoate (190 mg, 0.183 mmol) in CH₂Cl₂ (2 mL) at 0° C. was added TFA (2 mL). The resulting mixture was stirred at room temperature for 2 h before it was concentrated under reduced pressure. The residue was azeotroped with CH₂Cl₂ (3×5 mL) under reduced pressure to provide crude methyl 3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(thiazol-4-yl)propanoate.

### Step 5:

To a solution of a diastereomeric mixture (ca. 1:1) of crude methyl 3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(thiazol-4-yl)propanoate in THF (2 mL) and H₂O (2 mL) at 0° C. was added LiOH monohydrate (150 mg). The resulting mixture was stirred at room temperature overnight before it was cooled to 0° C. and acidified to pH ˜6 with 1N HCl(aq) solution and concentrated under reduced pressure. The crude residue was purified by preparative reversed-phase HPLC to provide a diastereomeric mixture (ca. 1:1) of 3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(thiazol-4-yl)propanoic acid as a white solid.

¹H NMR (CD₃OD, 300 MHz) δ 9.05-9.07 (m, 1H), 8.46 (s, 0.5H), 8.25 (s, 0.5H), 7.71-7.75 (m, 1H), 7.22-7.58 (m, 9H), 6.00-6.02 (d, J=5.4 Hz, 0.5H) 5.93-5.95 (d, J=5.91 Hz, 0.5H), 4.73-4.76 (t, J=5.34, 5.16 Hz, 0.5H), 4.66-4.70 (t, J=5.19, 5.49 Hz, 0.5H), 4.36-4.40 (q, J=3.93, 4.26, 3.3 Hz, 1H), 4.18-4.23 (m, 1H), 3.66-3.93 (m, 3H), 3.48-3.54 (m, 1H); LC/MS [M+H]=609.1.

### Examples 2 & 3

### Synthesis of 4′-((S)-2-(((2R,3S,4R,5R)-5-(6-amino-2-chloro-9H-purin-9-yl)-3,4-dihydroxytetrahydrofuran-2-yl)methoxy)-2-carboxy-2-(thiazol-4-yl)ethyl)-[1,1′-biphenyl]-4-carboxylic acid and 4′-((R)-2-(((2R,3S,4R,5R)-5-(6-amino-2-chloro-9H-purin-9-yl)-3,4-dihydroxytetrahydrofuran-2-yl)methoxy)-2-carboxy-2-(thiazol-4-yl)ethyl)-[1,1′-biphenyl]-4-carboxylic acid

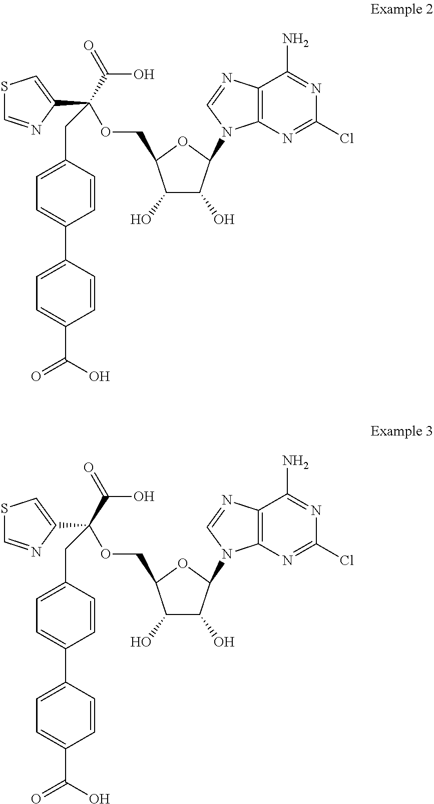 [SMILES: Nc1nc(Cl)nc2c1ncn2[C@@H]1O[C@H](CO[C@](Cc2ccc(-c3ccc(C(=O)O)cc3)cc2)(C(=O)O)c2cscn2)[C@@H](O)[C@H]1O ; Nc1nc(Cl)nc2c1ncn2[C@@H]1O[C@H](CO[C@@](Cc2ccc(-c3ccc(C(=O)O)cc3)cc2)(C(=O)O)c2cscn2)[C@@H](O)[C@H]1O]

Proceeding as described in Example 1 above but substituting 4-(bromomethyl)-1,1′-biphenyl with ethyl 4′-(bromomethyl)-[1,1′-biphenyl]-4-carboxylate provided a pair of diastereomeric products (ca. 1:1) which the stereo configuration was assigned arbitrarily. Both products were purified by preparative HPLC and isolated as white solids.

4′-((S)-2-(((2R,3S,4R,5R)-5-(6-amino-2-chloro-9H-purin-9-yl)-3,4-dihydroxytetrahydrofuran-2-yl)methoxy)-2-carboxy-2-(thiazol-4-yl)ethyl)-[1,1′-biphenyl]-4-carboxylic acid: ¹H NMR (CD₃OD, 300 MHz) δ 9.060-9.064 (d, J=1.38 Hz, 1H), 8.22 (s, 1H), 8.01-8.04 (d, J=8.13 Hz, 2H), 7.713-7.718 (d, J=1.44 Hz, 1H), 7.58-7.61 (d, J=8.25 Hz, 2H), 7.45-7.47 (d, J=7.95 Hz, 2H), 7.27-7.30 (d, J=8.04 Hz, 2H), 5.93-5.95 (d, J=5.79 Hz, 1H), 4.66-4.69 (t, J=4.77, 5.43 Hz, 1H), 4.37-4.39 (t, J=4.02, 4.26 Hz, 1H), 4.19-4.20 (m, 1H), 3.67-3.87 (m, 3H), 3.49-3.53 (m, 1H); LC/MS [M+H]=653.

4′-((R)-2-(((2R,3S,4R,5R)-5-(6-amino-2-chloro-9H-purin-9-yl)-3,4-dihydroxytetrahydrofuran-2-yl)methoxy)-2-carboxy-2-(thiazol-4-yl)ethyl)-[1,1′-biphenyl]-4-carboxylic acid: ¹H NMR (CD₃OD, 300 MHz) δ 9.08 (s, 1H), 8.48 (s, 1H), 7.98-8.01 (m, 2H), 7.78 (s, 1H), 7.27-7.52 (m, 6H), 6.00-6.02 (m, 1H), 4.72-4.76 (m, 1H), 4.39-4.40 (m, 1H), 4.23-4.25 (m, 1H), 3.69-3.96 (m, 3H), 3.48-3.51 (m, 11H); LC/MS [M+H]=653.

### Examples 4 & 5

### Synthesis of (S)-2-(((2R,3S,4R,5R)-5-(6-amino-2-chloro-9H-purin-9-yl)-3,4-dihydroxytetrahydrofuran-2-yl)methoxy)-3-(2′-(cyanomethyl)-[1,1′-biphenyl]-4-y)-2-(thiazol-4-yl)propanoic acid and (R)-2-(((2R,3S,4R,5R)-5-(6-amino-2-chloro-9H-purin-9-yl)-3,4-dihydroxytetrahydrofuran-2-yl)methoxy)-3-(2′-(cyanomethyl)-[1,1′-biphenyl]-4-yl)-2-(thiazol-4-yl)propanoic acid

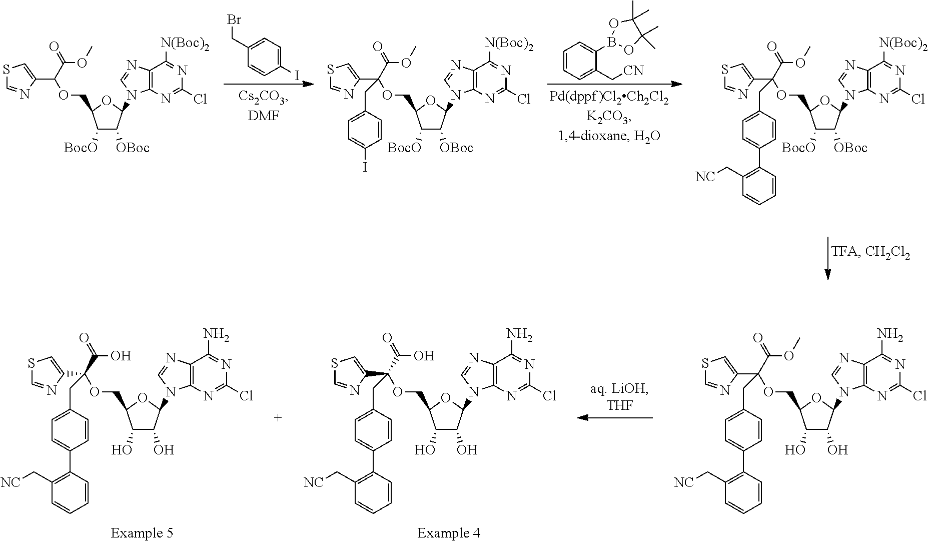

### Step 1:

To a solution of a diastereomeric mixture (ca. 1:1) of methyl 2-(((2R,3R,4R,5R)-5-(6-N,N′-(bis-tert-butoxycarbonyl)amino)-2-chloro-9H-purin-9-yl)-3,4-bis((tert-butoxy-carbonyl)oxy)tetra-hydrofuran-2-yl)methoxy)-2-(thiazol-4-yl)acetate (2 g, 2.33 mmol, 1 eq) in DMF (6 mL) at 25° C. was added Cs₂CO₃ (1.52 g, 4.67 mmol, 2 eq) and 4-iodobenzyl bromide (1.39 g, 4.67 mmol, 2 eq). The resulting mixture was stirred for 4 h before it was diluted with H₂O (25 mL) and extracted with EtOAc (30 mL). The organic layer was washed with H₂O (20 mL), brine (30 mL), dried (Na₂SO₄) and concentrated. The crude residue was purified by flash column chromatography on silica gel (0-40% EtOAc in hexanes) to provide a diastereomeric mixture (ca. 1:1) of methyl 2-(((2R,3R,4R,5R)-5-(6-N,N′-(bis-(tert-butoxy-carbonyl)amino)-2-chloro-9H-purin-9-yl)-3,4-bis((tert-butoxycarbonyl)oxy)tetrahydrofuran-2-yl)methoxy)-3-(4-iodophenyl)-2-(thiazol-4-yl)propanoate (2.45 g).

### Step 2:

To a solution of a diastereomeric mixture (ca. 1:1) of methyl 2-(((2R,3R,4R,5R)-5-(6-N,N′-(bis-(tert-butoxy-carbonyl)amino)-2-chloro-9H-purin-9-yl)-3,4-bis((tert-butoxy-carbonyl)oxy)tetrahydrofuran-2-yl)methoxy)-3-(4-iodophenyl)-2-(thiazol-4-yl)propanoate (200 mg, 0.186 mmole) and 2-cyanomethylphenylboronic acid, pinacol ester (91 mg, 0.373 mmole) in dioxane (2 mL) was added K₂CO₃ (129 mg, 0.932 mmole), Pd(dppf)Cl₂.CH₂Cl₂ (15 mg, 0.0186 mmole) and H₂O (0.7 ml). The mixture was degassed with bubbling argon through for 5 min and then was irradiated in a microwave reactor at 110° C. for 25 minutes. The reaction mixture then cooled to 25° C. before it was diluted with H₂O and extracted with EtOAc (3×5 mL). The combined organic layer was washed with brine (5 mL), dried over Na₂SO₄ then concentrated under reduced pressure. The resulting crude residue was purified by silica gel column chromatography (0-50% EtOAc in hexanes) to provide a pair of diastereomers of methyl 2-(((2R,3R,4R,5R)-5-(6-N,N′-(bis-(tert-butoxycarbonyl)amino)-2-chloro-9H-purin-9-yl)-3,4-bis((tert-butoxycarbonyl)oxy)tetrahydrofuran-2-yl)methoxy)-3-(2′-(cyanomethyl)-[1,1′-biphenyl]-4-yl)-2-(thiazol-4-yl)propanoate (ca. 1:1).

(S)-2-(((2R,3S,4R,5R)-5-(6-amino-2-chloro-9H-purin-9-yl)-3,4-dihydroxytetrahydrofuran-2-yl)methoxy)-3-(2′-(cyanomethyl)-[1,1′-biphenyl]-4-yl)-2-(thiazol-4-yl)propanoic acid: ¹H NMR (CD₃OD, 300 MHz) δ 9.05-9.06 (d, J=1.86 Hz, 1H), 8.26 (s, 1H), 7.69-7.70 (d, J=1.86 Hz, 1H), 7.11-7.49 (m, 8H), 5.94-5.96 (d, J=5.7 Hz, 1H), 4.63-4.67 (t, J=5.31 Hz, 1H), 4.35-4.38 (m, 1H), 4.19-4.22 (m, 1H), 3.82-3.87 (m, 2H), 3.52-3.75 (m, 4H); LC/MS [M+H]=648.

(R)-2-(((2R,3S,4R3R)-5-(6-amino-2-chloro-9H-purin-9-yl)-3,4-dihydroxytetrahydrofuran-2-yl)methoxy)-3-(2′-(cyanomethyl)-[1,1′-biphenyl]-4-yl)-2-(thiazol-4-yl)propanoic acid: ¹H NMR (MeOD, 300 MHz) δ 9.066-9.069 (d, J=1.05 Hz, 1H), 8.46 (s, 1H), 7.75-7.76 (d, J=1.47 Hz, 1H), 7.05-7.48 (m, 8H), 5.99-6.01 (d, J=5.31 Hz, 1H), 4.70-4.73 (t, J=5.16 Hz, 1H), 4.37-4.39 (t, J=4.26 Hz, 1H), 4.22-4.23 (m, 1H), 3.55-3.92 (m, 6H); LC/MS [M+H]=648.

### Examples 6 & 7

### Synthesis of (S)-2-(((2R,3S,4R,5R)-5-(6-amino-2-chloro-9H-purin-9-yl)-3,4-dihydroxytetrahydrofuran-2-yl)methoxy)-3-(2′-(carboxymethyl)-[1,1′-biphenyl]-4-yl)-2-(thiazol-4-yl)propanoic acid and (R)-2-(((2R,3S,4R,5R)-5-(6-amino-2-chloro-9H-purin-9-yl)-3,4-dihydroxytetrahydrofuran-2-yl)methoxy)-3-(2′-(carboxymethyl)-[1,1′-biphenyl]-4-yl)-2-(thiazol-4-yl)propanoic acid

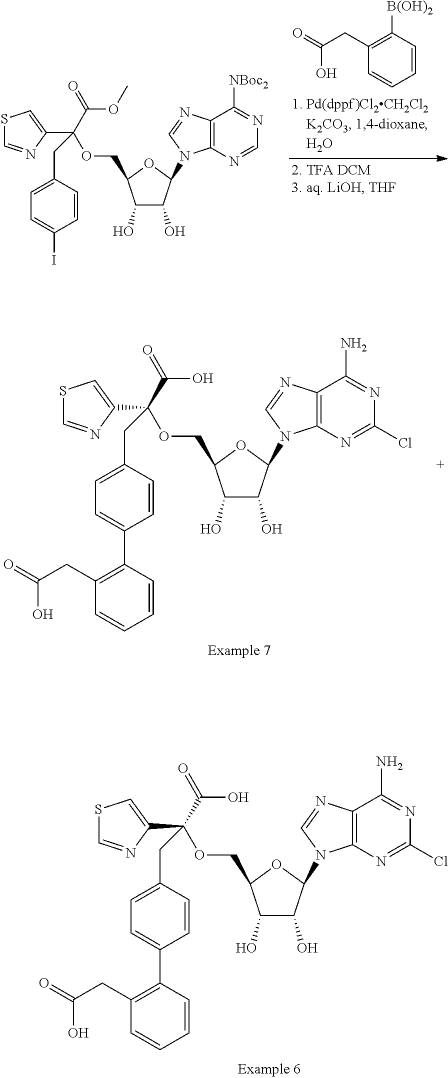

Proceeding as described in Examples 4 and 5 above but substituting 2-cyanomethylphenylboronic acid, pinacol ester with 2-(2-boronophenyl)acetic acid provided a pair of diastereomeric title products (ca. 1:1) which the stereo configuration was assigned arbitrarily. Both products were purified by preparative HPLC and isolated as white solids.

(S)-2-(((2R,3S,4R,5R)-5-(6-amino-2-chloro-9H-purin-9-yl)-3,4-dihydroxytetrahydrofuran-2-yl)methoxy)-3-(2′-(carboxymethyl)-[1,1′-biphenyl]-4-yl)-2-(thiazol-4-yl)propanoic acid: ¹H NMR (MeOD, 300 MHz) δ 9.03 (s, 1H), 8.30 (s, 1H), 7.63 (s, 1H), 7.05-7.28 (m, 8H), 5.95-5.96 (d, J=3.03 Hz, 1H), 4.67 (m, 1H), 4.38 (m, 1H), 4.22 (m, 1H), 3.46-3.89 (m, 6H); LC/MS [M+H]=667.1.

(R)-2-(((2R,3S,4R,5R)-5-(6-amino-2-chloro-9H-purin-9-yl)-3,4-dihydroxytetrahydrofuran-2-yl)methoxy)-3-(2′-(carboxymethyl)-[1,1′-biphenyl]-4-yl)-2-(thiazol-4-yl)propanoic acid: ¹H NMR (MeOD, 300 MHz) δ 9.05-9.06 (d, J=1.68 Hz, 1H), 8.50 (s, 1H), 7.73-7.74 (d, J=1.71 Hz, 1H), 7.05-7.29 (m, 8H), 5.99-6.01 (d, J=5.46 Hz, 1H), 4.74-4.77 (t, J=4.95, 5.22 Hz, 1H), 4.36-4.39 (t, J=3.69, 4.47 Hz, 1H), 4.22-4.23 (m, 1H), 3.49-3.92 (m, 6H); LC/MS [M+H]=667.1.

### Examples 8 & 9

### Synthesis of (S)-3-([1,1′-biphenyl]-4-yl)-2-(((2R,3R,4S,5R)-5-(6-amino-2-chloro-9H-purin-9-yl)-4-fluoro-3-hydroxytetrahydrofuran-2-yl)methoxy)-2-(thiazol-4-yl)propanoic acid and (R)-3-([1,1′-biphenyl]-4-yl)-2-(((2R,3R,4S,5R)-5-(6-amino-2-chloro-9H-purin-9-yl)-4-fluoro-3-hydroxytetrahydrofuran-2-yl)methoxy)-2-(thiazol-4-yl)propanoic acid

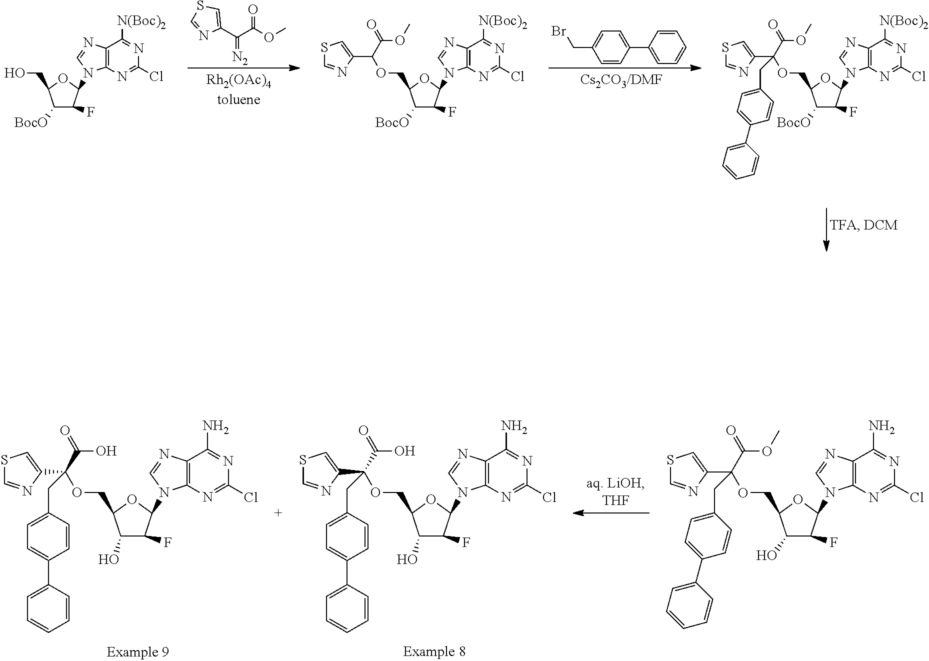

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate with tert-butyl (9-((2R,3S,4R,5R)-4-((tert-butoxycarbonyl)oxy)-3-fluoro-5-(hydroxymethyl)tetrahydrofuran-2-yl)-6-((tert-butoxy-carbonyl)amino)-2-chloro-9H-purin-6-yl)carbamate provided a pair of diastereomeric title products (ca. 1:1) which the stereo configuration was assigned arbitrarily. Both products were purified by preparative HPLC and isolated as white solids. (S)-3-([1,1′-biphenyl]-4-yl)-2-(((2R,3R,4S,5R)-5-(6-amino-2-chloro-9H-purin-9-yl)-4-fluoro-3-hydroxytetrahydrofuran-2-yl)methoxy)-2-(thiazol-4-yl)propanoic acid: ¹H NMR (CD₃OD, 300 MHz) δ 9.05 (s, 1H), 8.14 (s, 1H), 7.68 (s, 1H), 7.49-7.51 (d, J=7.71 Hz, 2H), 7.18-7.41 (m, 7H), 6.35-6.42 (dd, J=4.71, 15.3, 4.2 Hz, 1H), 5.03-5.23 (dt, J=2.88, 51.96, 3.15 Hz, 1H), 4.63-4.71 (dt, J=3.75, 17.61, 3.6 Hz, 1H), 4.09-4.16 (m, 1H), 3.92-3.97 (m, 1H), 3.60-3.84 (m, 3H); LC/MS [M+H]=611.1.

(R)-3-([1,1′-biphenyl]-4-yl)-2-(((2R,3R,4S,5R)-5-(6-amino-2-chloro-9H-purin-9-yl)-4-fluoro-3-hydroxytetrahydrofuran-2-yl)methoxy)-2-(thiazol-4-yl)propanoic acid: ¹H NMR (CD₃OD, 300 MHz) δ 9.04 (s, 1H), 8.35 (s, 1H), 8.27 (s, 1H), 7.71 (s, 1H), 7.25-7.43 (m, 8H), 6.36-6.41 (dd, J=4.74, 11.85 Hz, 1H), 5.06-5.27 (dt, J=4.32, 52.53 Hz, 1H), 4.66-4.76 (dt, J=9.27, 23.1 Hz, 1H), 4.05-4.10 (m, 1H), 3.66-3.81 (m, 4H); LC/MS [M+H]=611.1.

### Examples 10 & 11

### Synthesis of 4′-((S)-2-(((2R,3R,4S,5R)-5-(6-amino-2-chloro-9H-purin-9-yl)-4-fluoro-3-hydroxytetrahydrofuran-2-yl)methoxy)-2-carboxy-2-(thiazol-4-yl)ethyl)-[1,1′-biphenyl]-4-carboxylic acid and 4′-((R)-2-(((2R,3R,4S,5R)-5-(6-amino-2-chloro-9H-purin-9-yl)-4-fluoro-3-hydroxytetrahydrofuran-2-yl)methoxy)-2-carboxy-2-(thiazol-4-yl)ethyl)-[1,1′-biphenyl]-4-carboxylic acid

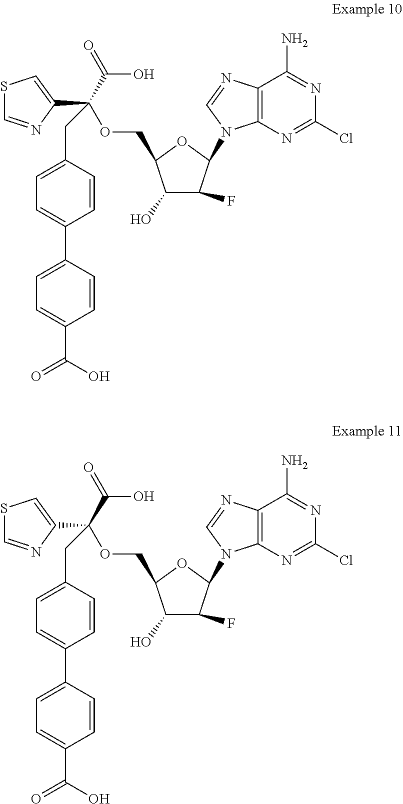 [SMILES: Nc1nc(Cl)nc2c1ncn2[C@@H]1O[C@H](CO[C@](Cc2ccc(-c3ccc(C(=O)O)cc3)cc2)(C(=O)O)c2cscn2)[C@@H](O)[C@@H]1F ; Nc1nc(Cl)nc2c1ncn2[C@@H]1O[C@H](CO[C@@](Cc2ccc(-c3ccc(C(=O)O)cc3)cc2)(C(=O)O)c2cscn2)[C@@H](O)[C@@H]1F]

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate and 4-(bromomethyl)-1,1′-biphenyl with tert-butyl (9-((2R,3S,4R,5R)-4-((tert-butoxycarbonyl)oxy)-3-fluoro-5-(hydroxylmethyl)-tetrahydrofuran-2-yl)-6-((tert-butoxycarbonyl)amino)-2-chloro-9H-purin-6-yl)carbamate and ethyl 4′-(bromomethyl)-[1,1′-biphenyl]-4-carboxylate provided a pair of diastereomeric title products (ca. 1:1) which the stereo configuration was assigned arbitrarily. Both products were purified by preparative HPLC and isolated as white solids.

4′-((S)-2-(((2R,3R,4S,5R)-5-(6-amino-2-chloro-9H-purin-9-yl)-4-fluoro-3-hydroxytetrahydro-furan-2-yl)methoxy)-2-carboxy-2-(thiazol-4-yl)ethyl)-[1,1′-biphenyl]-4-carboxylic acid: ¹H NMR (MeOD, 300 MHz) δ 9.05-9.06 (d, J=1.89 Hz, 1H), 8.03-8.11 (m, 3H), 7.61-7.68 (m, 3H), 7.44-7.47 (d, J=8.16 Hz, 2H), 7.22-7.25 (d, J=8.28 Hz, 2H), 6.35-6.42 (dd, J=4.35, 15.27 Hz, 1H), 5.03-5.23 (dt, J=3.18, 52.77 Hz, 1H), 4.63-4.71 (dt, J=3.78, 17.79 Hz, 1H), 4.12-4.16 (q, J=4.47, 4.41 Hz, 1H), 3.94-3.99 (m, 1H), 3.62-3.84 (m, 3H); LC/MS [M+H]=655.1.

4′-((R)-2-(((2R,3R,4S,5R)-5-(6-amino-2-chloro-9H-purin-9-yl)-4-fluoro-3-hydroxytetrahydro-furan-2-yl)methoxy)-2-carboxy-2-(thiazol-4-yl)ethyl)-[1,1′-biphenyl]-4-carboxylic acid: ¹H NMR (MeOD, 300 MHz) δ 9.07 (s, 1H), 8.34 (s, 1H), 8.00-8.03 (d, J=8.4 Hz, 2H), 7.76 (s, 1H), 7.50-7.52 (d, J=8.19 Hz, 2H), 7.28-7.38 (q, J=8.01, 22.1 Hz, 4H), 6.38-6.43 (dd, J=4.77, 11.43 Hz, 1H), 5.05-5.28 (dt, J=4.14, 52.71 Hz, 1H), 4.67-4.78 (m, 1H), 4.08-4.11 (m, 1H), 3.66-3.87 (m, 4H); LC/MS [M+H]=655.1.

### Example 12

### Synthesis of 2-(((2R3R,4S,5R)-5-(6-amino-2-chloro-9H-purin-9-yl)-4-fluoro-3-hydroxytetrahydrofuran-2-yl)methoxy)-3-(2′-cyano-[1,1′-biphenyl]-4-yl)-2-(thiazol-4-yl)propanoic acid

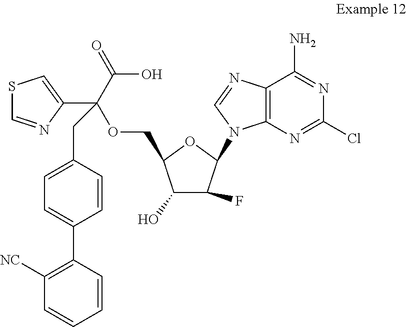 [SMILES: N#Cc1ccccc1-c1ccc(CC(OC[C@H]2O[C@@H](n3cnc4c(N)nc(Cl)nc43)[C@@H](F)[C@@H]2O)(C(=O)O)c2cscn2)cc1]

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate and 4-(bromomethyl)-1,1′-biphenyl with tert-butyl (9-((2R,3S,4R,5R)-4-((tert-butoxycarbonyl)oxy)-3-fluoro-5-(hydroxymethyl)-tetrahydrofuran-2-yl)-6-((tert-butoxycarbonyl)amino)-2-chloro-9H-purin-6-yl)carbamate and 4′-(bromomethyl)-[1,1′-biphenyl]-2-carbonitrile provided the title compound as a mixture of diastereomers (ca. 1:1) and isolated as a white solid.

¹H NMR (MeOD, 300 MHz) δ 9.06 (m, 1H), 8.32 (s, 0.5H), 8.11 (s, 0.5H), 7.62-7.79 (m, 3H), 7.25-7.50 (m, 6H), 6.35-6.43 (m, 1H), 5.03-5.26 (m, 1H), 4.62-4.76 (m, 1H), 4.09-4.17 (m, 1H), 3.65-4.00 (m, 4H); LC/MS [M+H]=636.1.

### Example 13

### Synthesis of 4′-(2-(((2R,3R,4S,5R)-5-(6-amino-2-chloro-9H-purin-9-yl)-4-fluoro-3-hydroxytetrahydrofuran-2-yl)methoxy)-2-carboxy-2-(thiazol-4-yl)ethyl)-[1,1′-biphenyl]-2-carboxylic acid

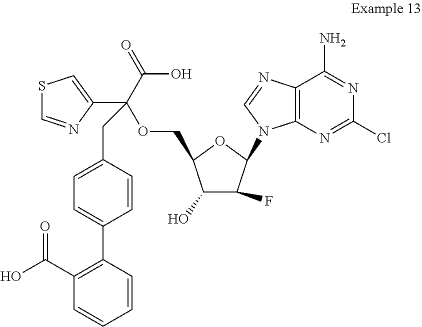 [SMILES: Nc1nc(Cl)nc2c1ncn2[C@@H]1O[C@H](COC(Cc2ccc(-c3ccccc3C(=O)O)cc2)(C(=O)O)c2cscn2)[C@@H](O)[C@@H]1F]

Proceeding as described in Example 1 above but substituting tert-butyl (9-((2R,3R4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate and 4-(bromomethyl)-1,1′-biphenyl with tert-butyl (9-((2R,3S,4R,5R)-4-((tert-butoxycarbonyl)oxy)-3-fluoro-5-(hydroxymethyl)-tetrahydrofuran-2-yl)-6-((tert-butoxycarbonyl)amino)-2-chloro-9H-purin-6-yl)carbamate and methyl 4′-(bromomethyl)-[1,1′-biphenyl]-2-carboxylate provided the title compound as a mixture of diastereomers (ca. 1:1) and isolated as a white solid.

¹H NMR (MeOD, 300 MHz) δ 9.03-9.05 (m, 1H), 8.343-8.347 (d, J=1.41 Hz, 0.5H), 8.17-8.18 (d, J=1.86 Hz, 0.5H), 7.52-7.76 (m, 2H), 7.07-7.50 (m, 7H), 6.36-6.44 (m, 1H), 5.02-5.26 (m, 1H), 4.61-4.72 (m, 1H), 4.09-4.16 (m, 1H), 3.61-3.97 (m, 4H); LC/MS [M+H]=655.1.

### Example 14

### Synthesis of 2-(((2R3R,4S,5R)-5-(6-amino-2-chloro-9H-purin-9-yl)-4-fluoro-3-hydroxytetrahydrofuran-2-yl)methoxy)-2-(thiazol-4-yl)acetic acid

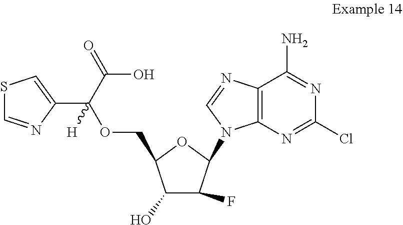 [SMILES: Nc1nc(Cl)nc2c1ncn2[C@@H]1O[C@H](COC(C(=O)O)c2cscn2)[C@@H](O)[C@@H]1F]

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate with tert-butyl (9-((2R,3S,4R,5R)-4-((tert-butoxycarbonyl)oxy)-3-fluoro-5-(hydroxymethyl)tetrahydrofuran-2-yl)-6-((tert-butoxycarbonyl)amino)-2-chloro-9H-purin-6-yl)carbamate and without the alkylation with 4-(bromomethyl)-1,1′-biphenyl provided the title compound as a mixture of diastereomers (ca. 1:1) and isolated as a white solid.

¹H NMR (MeOD, 300 MHz) δ 9.020-9.024 (m, 1H), 8.35-8.44 (d, J 28.26 Hz, 1H), 7.68-7.72 (dd, J=1.86, 10.53 Hz, 1H), 6.39-6.45 (dd, J=4.5, 12.81 Hz, 1H), 5.36 (s, 1H), 5.08-5.29 (m, 1H), 4.60-4.69 (m, 1H), 4.10-4.17 (m, 1H), 3.78-4.00 (m, 2H); LC/MS [M+H]=445.0.

### Examples 15 & 16

### Synthesis of 4-((S)-2-(((2R,3R,4S,5R)-5-(6-amino-2-chloro-9H-purin-9-yl)-4-fluoro-3-hydroxytetrahydrofuran-2-yl)methoxy)-2-carboxy-2-(thiazol-4-yl)ethyl)benzoic acid and 4-((R)-2-(((2R,3R,4S,5R)-5-(6-amino-2-chloro-9H-purin-9-yl)-4-fluoro-3-hydroxytetrahydrofuran-2-yl)methoxy)-2-carboxy-2-(thiazol-4-yl)ethyl)benzoic acid

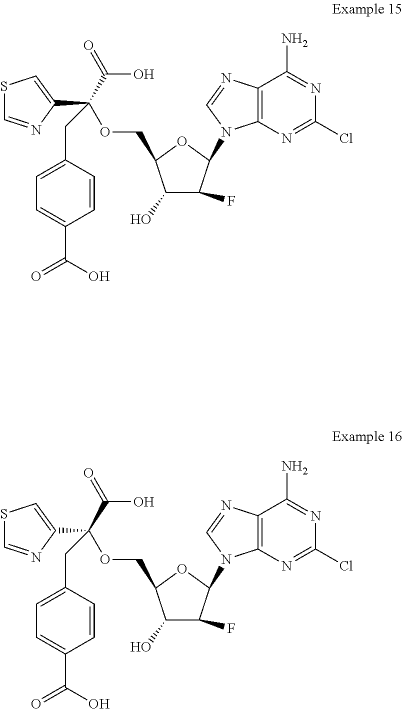 [SMILES: Nc1nc(Cl)nc2c1ncn2[C@@H]1O[C@H](CO[C@](Cc2ccc(C(=O)O)cc2)(C(=O)O)c2cscn2)[C@@H](O)[C@@H]1F ; Nc1nc(Cl)nc2c1ncn2[C@@H]1O[C@H](CO[C@@](Cc2ccc(C(=O)O)cc2)(C(=O)O)c2cscn2)[C@@H](O)[C@@H]1F]

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate and 4-(bromomethyl)-1,1′-biphenyl with tert-butyl (9-((2R,3S,4R,5R)-4-((tert-butoxycarbonyl)oxy)-3-fluoro-5-(hydroxymethyl)-tetrahydrofuran-2-yl)-6-((tert-butoxycarbonyl)amino)-2-chloro-9H-purin-6-yl)carbamate and methyl 4-(bromomethyl)benzoate provided a pair of diastereomeric title products (ca. 1:1) which the stereo configuration was assigned arbitrarily. Both products were purified by preparative HPLC and isolated as white solids.

4-((S)-2-(((2R,3R,4S,5R)-5-(6-amino-2-chloro-9H-purin-9-yl)-4-fluoro-3-hydroxytetrahydro-furan-2-yl)methoxy)-2-carboxy-2-(thiazol-4-yl)ethyl)benzoic acid: ¹H NMR (MeOD, 300 MHz) δ 9.04-9.05 (d, J=1.89 Hz, 1H), 8.09-8.10 (d, J=1.89 Hz, 1H), 7.79-7.81 (d, J=8.19 Hz, 2H), 7.64-7.65 (d, J=1.95 Hz, 1H), 7.21-7.24 (d, J=8.16 Hz, 2H), 6.35-6.42 (dd, J=4.17, 15.72 Hz, 1H), 5.02-5.21 (dt, J=3.36, 52.2 Hz, 1H), 4.59-4.67 (dt, J=3.75, 16.86 Hz, 1H), 4.09-4.16 (m, 11H), 3.92-3.97 (m, 1H), 3.62-3.85 (m, 3H); LC/MS [M+H]=579.0.

4-((R)-2-(((2R,3R,4S,5R)-5-(6-amino-2-chloro-9H-purin-9-yl)-4-fluoro-3-hydroxytetrahydro-furan-2-yl)methoxy)-2-carboxy-2-(thiazol-4-yl)ethyl)benzoic acid: ¹H NMR (MeOD, 300 MHz) δ 9.05 (s, 1H), 8.27 (s, 1H), 7.69-7.75 (m, 3H), 7.25-7.27 (d, J=8.16 Hz, 2H), 6.38-6.44 (dd, J=4.35, 13.35 Hz, 1H), 5.05-5.25 (dt, J=4.11, 52.71 Hz, 1H), 4.64-4.73 (dt, J=4.29, 18 Hz, 1H), 4.07-4.12 (m, 1H), 3.62-3.86 (m, 4H); LC/MS [M+H]=579.1.

### Example 17

### Synthesis of 3-(2-(((2R,3R,4S,5R)-5-(6-amino-2-chloro-9H-purin-9-yl)-4-fluoro-3-hydroxytetrahydrofuran-2-yl)methoxy)-2-carboxy-2-(thiazol-4-yl)ethyl)benzoic acid

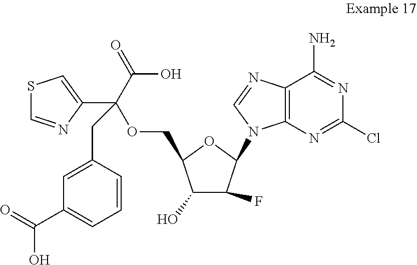 [SMILES: Nc1nc(Cl)nc2c1ncn2[C@@H]1O[C@H](COC(Cc2cccc(C(=O)O)c2)(C(=O)O)c2cscn2)[C@@H](O)[C@@H]1F]

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate and 4-(bromomethyl)-1,1′-biphenyl with tert-butyl (9-((2R,3S,4R,5R)-4-((tert-butoxycarbonyl)oxy)-3-fluoro-5-(hydroxymethyl)-tetrahydrofuran-2-yl)-6-((tert-butoxycarbonyl)amino)-2-chloro-9H-purin-6-yl)carbamate and methyl 3-(bromomethyl)benzoate provided the title compound as a mixture of diastereomers (ca. 1:1) and isolated as a white solid.

¹H NMR (MeOD, 300 MHz) δ 9.04-9.06 (m, 1H), 8.291-8.296 (d, J=1.41 Hz, 0.4H), 8.10-8.11 (d, J=1.86 Hz, 0.6H), 7.63-7.85 (m, 3H), 7.17-7.42 (m, 2H), 6.35-6.43 (m, 1H), 5.01-5.25 (m, 1H), 4.60-4.76 (m, 1H), 4.08-4.16 (m, 1H), 3.62-3.97 (m, 4H); LC/MS [M+H]=579.1.

### Example 18

### Synthesis of 2-(((2R3R,4S,5R)-5-(6-amino-2-chloro-9H-purin-9-yl)-4-fluoro-3-hydroxytetrahydrofuran-2-yl)methoxy)-3-(2′-(carboxymethyl)-[1,1′-biphenyl]-4-yl)-2-(thiazol-4-yl)propanoic acid

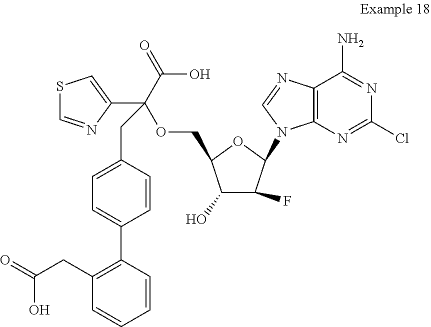 [SMILES: Nc1nc(Cl)nc2c1ncn2[C@@H]1O[C@H](COC(Cc2ccc(-c3ccccc3CC(=O)O)cc2)(C(=O)O)c2cscn2)[C@@H](O)[C@@H]1F]

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxy-carbonyl)amino)-2-chloro-9H-purin-6-yl)carbamate and 4-(bromomethyl)-1,1′-biphenyl with tert-butyl (9-((2R,3S,4R,5R)-4-((tert-butoxycarbonyl)oxy)-3-fluoro-5-(hydroxymethyl)-tetrahydrofuran-2-yl)-6-((tert-butoxycarbonyl)amino)-2-chloro-9H-purin-6-yl)carbamate and 22-(2-boronophenyl)acetic acid provided the title compound as a mixture of diastereomers (ca. 1:1) and isolated as a white solid.

¹H NMR (MeOD, 300 MHz) δ 9.04 (s, 1H), 8.35 (s, 0.5H), 8.13 (s, 0.5H), 7.64-7.72 (m, 1H), 7.05-7.30 (m, 8H), 6.35-6.41 (m, 1H), 5.02-5.25 (m, 1H), 4.62-4.73 (m, 1H), 4.10-4.17 (m, 1H), 3.62-3.84 (m, 4H), 3.47-3.49 (d, J=5.37 Hz, 2H); LC/MS [M+H]=669.1.

### Examples 19 & 20

### Synthesis of (S)-2-(((2R,3R4S,5R)-5-(6-amino-2-chloro-9H-purin-9-yl)-4-fluoro-3-hydroxytetrahydrofuran-2-yl)methoxy)-3-(4-(2-methoxypyridin-3-yl)phenyl)-2-(thiazol-4-yl)propanoic acid and (R)-2-(((2R,3R,4S,5R)-5-(6-amino-2-chloro-9H-purin-9-yl)-4-fluoro-3-hydroxytetrahydrofuran-2-yl)methoxy)-3-(4-(2-methoxypyridin-3-yl)phenyl)-2-(thiazol-4-yl)propanoic acid

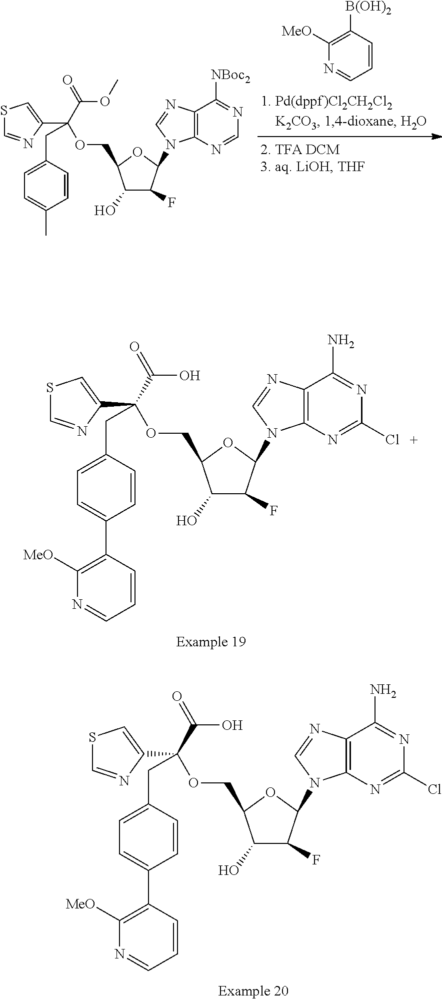

Proceeding as described in Examples 4 and 5 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate and 2-cyanomethylphenylboronic acid, pinacol ester with tert-butyl (9-((2R,3S,4R,5R)-4-((tert-butoxycarbonyl)-oxy)-3-fluoro-5-(hydroxymethyl)tetrahydrofuran-2-yl)-6-((tert-butoxycarbonyl)amino)-2-chloro-9H-purin-6-yl)carbamate and (2-methoxypyridin-3-yl)boronic acid provided a pair of diastereomeric title products (ca. 1:1) which the stereo configuration was assigned arbitrarily. Both products were purified by preparative HPLC and isolated as white solids.

(S)-2-(((2R,3R,4S,5R)-5-(6-amino-2-chloro-9H-purin-9-yl)-4-fluoro-3-hydroxytetrahydro-furan-2-yl)methoxy)-3-(4-(2-methoxypyridin-3-yl)phenyl)-2-(thiazol-4-yl)propanoic acid: ¹H NMR (MeOD, 300 MHz) δ 9.051-9.058 (d, J=2.01 Hz, 1H), 8.05-8.10 (m, 2H), 7.68-7.69 (d, J=1.98 Hz, 1H), 7.58-7.61 (dd, J=1.89, 7.29 Hz, 1H), 7.32-7.35 (d, J=8.31 Hz, 2H), 7.16-7.19 (d, J=8.25 Hz, 2H), 6.96-7.00 (m, 1H), 6.35-6.41 (dd, J=3.99, 14.97 Hz, 1H), 5.03-5.22 (dt, J=3.21, 52.83 Hz, 1H), 4.62-4.70 (m, 1H), 4.11-4.15 (q, J=4.35 Hz, 1H), 3.60-3.96 (m, 7H); LC/MS [M+H]=642.1.

(R)-2-(((2R,3R,4S,5R)-5-(6-amino-2-chloro-9H-purin-9-yl)-4-fluoro-3-hydroxytetrahydro-furan-2-yl)methoxy)-3-(4-(2-methoxypyridin-3-yl)phenyl)-2-(thiazol-4-yl)propanoic acid: ¹H NMR (MeOD, 300 MHz) δ 9.061-9.067 (d, J=1.92 Hz, 1H), 8.34 (s, 1H), 8.01-8.03 (dd, J=1.92, 5.01 Hz, 1H), 7.75-7.76 (d, J=1.92 Hz, 1H), 7.45-7.48 (dd. J=1.92, 7.38 Hz, 1H), 7.22-7.29 (q, J=8.34, 4.29 Hz, 4H), 6.90-6.94 (m, 1H), 6.38-6.43 (dd, J=4.47, 11.52 Hz, 1H), 5.07-5.28 (dt, J=4.41, 52.56 Hz, 1H), 4.67-4.77 (dt, J=4.74, 18.36 Hz, 1H), 4.08-4.11 (q, J=3.72, 5.1 Hz, 1H), 3.64-3.87 (m, 7H); LC/MS [M+H]=642.1.

### Examples 21 & 22

### Synthesis of (S)-2-(((2R,3R,4S,5R)-5-(6-amino-2-chloro-9H-purin-9-yl)-4-fluoro-3-hydroxytetrahydrofuran-2-yl)methoxy)-3-(4-(2-oxo-1,2-dihydropyridin-3-yl)phenyl)-2-(thiazol-4-yl)propanoic acid and (R)-2-(((2R,3R,4S,5R)-5-(6-amino-2-chloro-9H-purin-9-yl)-4-fluoro-3-hydroxytetrahydrofuran-2-yl)methoxy)-3-(4-(2-oxo-1,2-dihydropyridin-3-yl)phenyl)-2-(thiazol-4-yl)propanoic acid

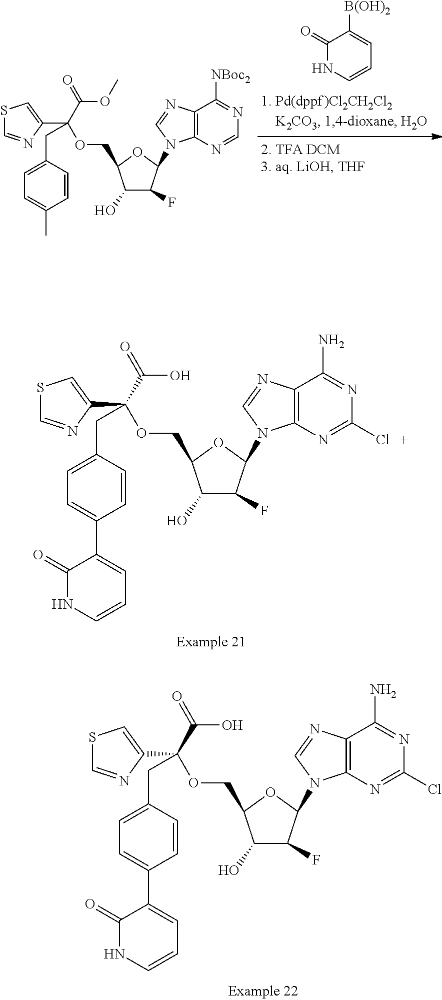

Proceeding as described in Examples 4 and 5 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate and 2-cyanomethylphenylboronic acid, pinacol ester with tert-butyl (9-((2R,3S,4R,5R)-4-((tert-butoxycarbonyl)-oxy)-3-fluoro-5-(hydroxymethyl)tetrahydrofuran-2-yl)-6-((tert-butoxycarbonyl)amino)-2-chloro-9H-purin-6-yl)carbamate and (2-oxo-1,2-dihydropyridin-3-yl)boronic acid provided a pair of diastereomeric title products (ca. 1:1) which the stereo configuration was assigned arbitrarily. Both products were purified by preparative HPLC and isolated as white solids.

(S)-2-(((2R,3R,4S,5R)-5-(6-amino-2-chloro-9H-purin-9-yl)-4-fluoro-3-hydroxytetrahydro-furan-2-yl)methoxy)-3-(4-(2-oxo-1,2-dihydropyridin-3-yl)phenyl)-2-(thiazol-4-yl)propanoic acid: ¹H NMR (MeOD, 300 MHz) δ 9.04-9.05 (d, J=2.01 Hz, 1H), 8.13-8.14 (d, J=1.95 Hz, 1H), 7.66-7.67 (d, J=1.98 Hz, 1H), 7.59-7.62 (dd, J=2.01, 7.05 Hz, 1H), 7.45-7.48 (d, J=8.19 Hz, 2H), 7.36-7.39 (dd, J=1.98, 6.36 Hz, 1H), 7.16-7.19 (d, J=8.25 Hz, 2H), 6.35-6.47 (m, 2H), 5.02-5.21 (dt, J=3.54, 52.38 Hz, 1H), 4.60-4.68 (dt, J=3.6, 18.06 Hz, 1H), 4.10-4.15 (q, J=4.89 Hz, 1H), 3.60-3.95 (m, 4H); LC/MS [M+H]=628.0.

(R)-2-(((2R,3R,4S,5R)-5-(6-amino-2-chloro-9H-purin-9-yl)-4-fluoro-3-hydroxytetrahydro-furan-2-yl)methoxy)-3-(4-(2-oxo-1,2-dihydropyridin-3-yl)phenyl)-2-(thiazol-4-yl)propanoic acid: ¹H NMR (MeOD, 300 MHz) δ 9.04-9.05 (d, J=1.95 Hz, 1H), 8.321-8.326 (d, J=1.53 Hz, 1H), 7.71-7.72 (d, J=1.98 Hz, 1H), 7.50-7.53 (dd, J=1.95, 6.96 Hz, 1H), 7.33-7.42 (m, 3H), 7.20-7.23 (d, J=8.22 Hz, 2H), 6.37-6.44 (m, 2H), 5.05-5.25 (dt, J=4.53, 52.5 Hz, 1H), 4.64-4.73 (dt, J=9.15, 13.29 Hz, 1H), 4.06-4.10 (q, J=3.81, 4.74 Hz, 2H), 3.63-3.83 (m, 3H); LC/MS [M+H]=628.0.

### Example 23

### Synthesis of 2-(((2R,3R,4S,5R)-5-(6-butoxy-2-chloro-9H-purin-9-yl)-4-fluoro-3-hydroxytetrahydrofuran-2-yl)methoxy)-3-phenyl-2-(thiazol-4-yl)propanoic acid

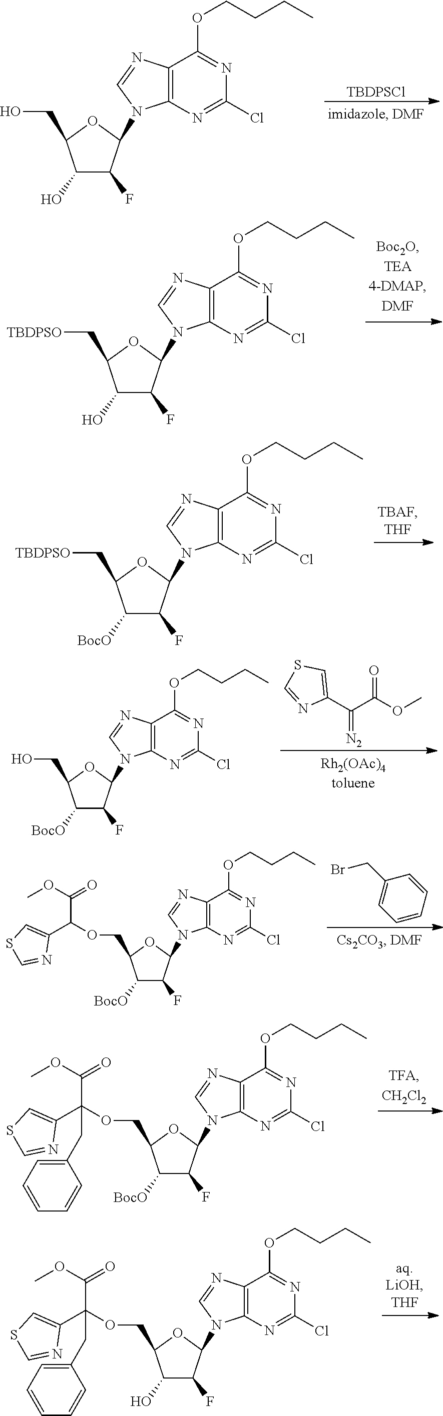

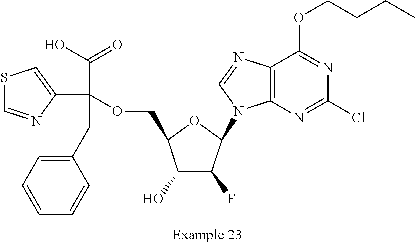 [SMILES: CCCCOc1nc(Cl)nc2c1ncn2[C@@H]1O[C@H](COC(Cc2ccccc2)(C(=O)O)c2cscn2)[C@@H](O)[C@@H]1F]

### Step 1:

To a solution of (2R,3R,4S,5R)-5-(6-butoxy-2-chloro-9H-purin-9-yl)-4-fluoro-2-(hydroxymethyl)tetrahydrofuran-3-ol (377 mg, 1.045 mmol) which was prepared according to the procedure described previously (WO2018049145 and WO2018119284) in DMF (2 mL) at 0° C. under argon atmosphere was added imidazole (179 mg, 2.62 mmol) and followed by TBDPSCl (312 uL, 1.2 mmol). The reaction mixture was stirred for 2 hours at 0° C. and then allowed to warm up to room temperature and stirred for 18 h. The solvent was removed under reduced pressure and the residue was diluted with H₂O (10 mL) and extracted with EtOAc (3×25 mL). The combined organic layer was washed further with H₂O (30 mL), brine (30 mL), dried over Na₂SO₄ and concentrated. The crude residue was purified by flash column chromatography on silica gel (0-40% EtOAc in hexanes) to provide (2R,3R,4S,5R)-5-(6-butoxy-2-chloro-9H-purin-9-yl)-2-(((tert-butyldiphenylsilyl)oxy)methyl)-4-fluorotetra-hydrofuran-3-ol (650 mg).

### Step 2:

To a solution of (2R,3R,4S,5R)-5-(6-butoxy-2-chloro-9H-purin-9-yl)-2-(((tert-butyl-diphenylsilyl)oxy)methyl)-4-fluorotetrahydrofuran-3-ol (650 mg, 1.085 mmol) in dry DMF (5 mL) under argon atmosphere at 0° C. was added Et₃N (166 uL, 1.193 mmol), 4-DMAP (22 mg, 0.1807 mmol) and followed by dropwise addition of a solution of Boc₂O (249 mg, 1.139 mmol) in dry DMF (1 mL). The reaction mixture was stirred for 1 h at 0° C. and then at 25° C. for 18 h before it was concentrated under reduced pressure. The residue was purified by silica gel column chromatography (0-40% EtOAc in hexanes) to provide (2R3R4S,5R)-5-(6-butoxy-2-chloro-9H-purin-9-yl)-2-(((tert-butyldiphenylsilyl)oxy)-methyl)-4-fluorotetrahydro-furan-3-yl tert-butyl carbonate (600 mg).

### Step 3:

To a solution of (2R,3R,4S,5R)-5-(6-butoxy-2-chloro-9H-purin-9-yl)-2-(((tert-butyldiphenyl-silyl)oxy)-methyl)-4-fluorotetrahydrofuran-3-yl tert-butyl carbonate (600 mg, 0.858 mmole) was dissolved in dry THF (10 mL) was added a solution of TBAF (1.3 mL, 1.287 mmol, 1 M in THF) dropwise. The reaction mixture was stirred 18 h before it was evaporated to dryness. The residue was purified by silica gel column chromatography (0-40% EtOAc in hexanes) to provide (2R,3R4S,5R)-5-(6-butoxy-2-chloro-9H-purin-9-yl)-4-fluoro-2-(hydroxymethyl)-tetrahydrofuran-3-yl tert-butyl carbonate (301 mg).

### Step 4:

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate with (2R,3R4S,5R)-5-(6-butoxy-2-chloro-9H-purin-9-yl)-4-fluoro-2-(hydroxymethyl)tetra-hydrofuran-3-yl tert-butyl carbonate provided the title compound as a mixture of diastereomers (ca. 1:1) and isolated as a white solid.

¹H NMR (MeOD, 300 MHz) δ 9.03-9.04 (d, J=1.68 Hz, 1H), 8.45-8.46 (d, J=1.74 Hz, 0.5H), 8.30-8.31 (d, J=1.8 Hz, 0.5H), 7.63-7.66 (dd, J=1.98, 7.44 Hz, 1H), 7.08-7.16 (m, 5H), 6.44-6.52 (dt, J=3.9, 14.88 Hz, 1H), 5.06-5.26 (m, 1H), 4.79-4.59 (m, 3H), 4.10-4.17 (m, 1H), 3.55-3.93 (m, 4H), 1.83-1.92 (m, 2H), 1.50-1.62 (m, 2H), 1.00-1.05 (t, J=7.35 Hz, 3H); LC/MS [M+H]=592

### Example 24

### Synthesis of 2-(((2R,3R4S,5R)-5-(2-chloro-6-(propylamino)-9H-purin-9-yl)-4-fluoro-3-hydroxytetrahydrofuran-2-yl)methoxy)-3-phenyl-2-(thiazol-4-yl)propanoic acid

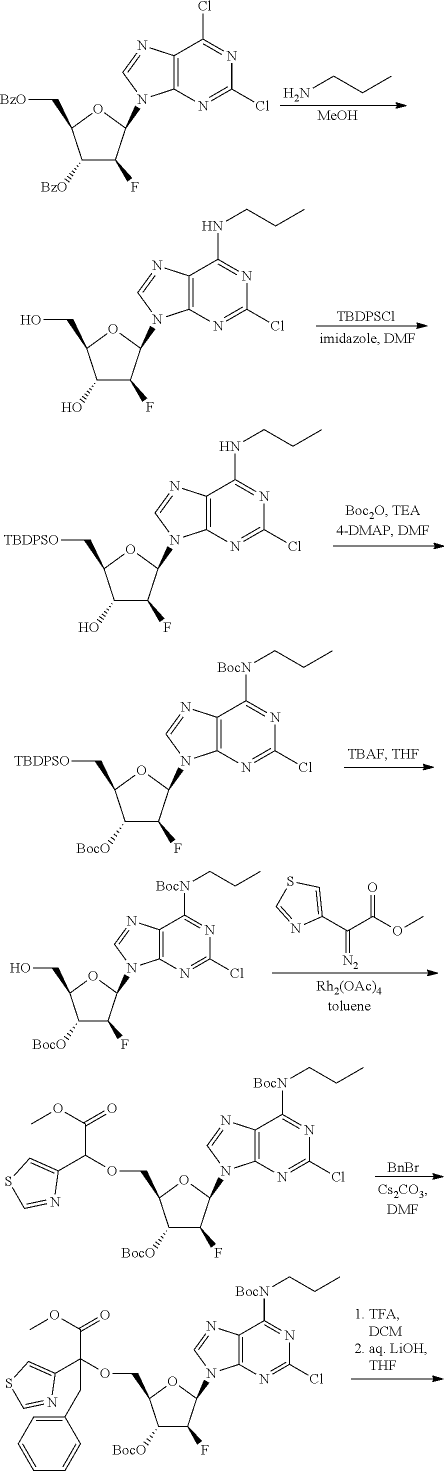

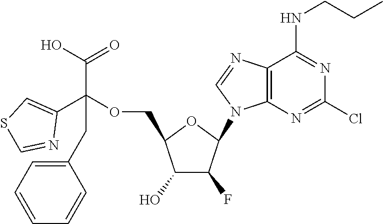 [SMILES: CCCNc1nc(Cl)nc2c1ncn2[C@@H]1O[C@H](COC(Cc2ccccc2)(C(=O)O)c2cscn2)[C@@H](O)[C@@H]1F]

### Step 1:

A mixture of ((2R,3R,4S,5R)-3-(benzoyloxy)-5-(2,6-dichloro-9H-purin-9-yl)-4-fluorotetrahydrofuran-2-yl)methyl benzoate (5.00 g, 0.94 mmol, 1 eq) and propylamine (4.44 g, 75.28 mmol, 8 eq) in MeOH (50 mL) was stirred at 25° C. for 5 h before it was concentrated. The crude was dissolved in a mixture of 1N aq. LiOH (20 mL) and THF (10 ml). The mixture was stirred for 1 h before the organic volatile was removed. The aq. layer was cooled to 0° C. and acidified to pH ˜6 with 2N aq. HCl solution. The precipitate was collected by suction filtration and dried to provide crude (2R,3R,4S,5R)-5-(2-chloro-6-(propylamino)-9H-purin-9-yl)-4-fluoro-2-(hydroxymethyl)tetrahydrofuran-3-ol (1.20 g).

### Step 2:

To a solution of crude (2R3R,4S,5R)-5-(2-chloro-6-(propylamino)-9H-purin-9-yl)-4-fluoro-2-(hydroxymethyl)tetrahydrofuran-3-ol (1.20 g, 3.47 mmol, 1 eq) in DMF (8 mL) at 0° C. under argon atmosphere was added imidazole (709 mg, 10.41 mmol, 3 eq) and TBDPSCl (1.08 mL, 4.16 mmol, 1.2 eq). The reaction mixture was stirred at 25° C. for 5 h before it was diluted with H₂O (10 mL) and extracted with EtOAc (100 mL). The organic layer was washed further with H₂O (2×30 mL), brine (30 mL), dried over Na₂SO₄ and concentrated. The crude residue was purified by flash column chromatography on silica gel (10-80% EtOAc in hexanes) to provide (2R,3R,4S,5R)-2-(((tert-butyldiphenylsilyl)oxy)methyl)-5-(2-chloro-6-(propylamino)-9H-purin-9-yl)-4-fluorotetrahydrofuran-3-ol (780 mg).

### Step 3:

To a solution of (2R,3R,4S,5R)-2-(((tert-butyldiphenylsilyl)oxy)methyl)-5-(2-chloro-6-(propylamino)-9H-purin-9-yl)-4-fluorotetrahydrofuran-3-ol (700 mg, 1.20 mmol, 1 eq) in dry THF (5 mL) under argon atmosphere at 0° C. was added Et₃N (668 uL, 4.79 mmol, 4 eq), 4-DMAP (60 mg) and Boc₂O (1.05 mg, 4.79 mmol). The reaction mixture was stirred at 25° C. for 4 h before it was concentrated under reduced pressure. The residue was purified by silica gel column chromatography (10-40% EtOAc in hexanes) to provide tert-butyl (9-((2R,3S,4R,5R)-4-((tert-butoxycarbonyl)oxy)-5-(((tert-butyldiphenylsilyl)oxy)methyl)-3-fluorotetrahydrofuran-2-yl)-2-chloro-9H-purin-6-yl)(propyl)carbamate (780 mg).

### Step 4:

To a solution of tert-butyl (9-((2R,3S,4R,5R)-4-((tert-butoxycarbonyl)oxy)-5-(((tert-butyldiphenylsilyl)oxy)methyl)-3-fluorotetrahydrofuran-2-yl)-2-chloro-9H-purin-6-yl)(propyl)carbamate (700 mg, 0.99 mmole, 1 eq) was dissolved in dry THF (10 mL) at 25° C. was added a solution of TBAF (2.0 mL, 1.98 mmol, 1 M in THF) dropwise. The reaction mixture was stirred 4 h before it was diluted with water (10 mL) and EtOAc (40 mL). The organic layer was washed with brine (10 mL), dried (Na₂SO₄) and concentrated. The residue was purified by silica gel column chromatography (10-50% EtOAc in hexanes) to provide (2R,3R4S,5R)-5-(6-butoxy-2-chloro-9H-purin-9-yl)-4-fluoro-2-(hydroxymethyl)-tetrahydrofuran-3-yl tert-butyl carbonate (301 mg).

### Step 5:

Proceeding as described in Example 1 above but substituting tert-butyl (9-((2R3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate with (2R3R,4S,5R)-5-(6-butoxy-2-chloro-9H-purin-9-yl)-4-fluoro-2-(hydroxymethyl)-tetrahydrofuran-3-yl tert-butyl carbonate provided the title compound as a mixture of diastereomers (ca. 1:1) and isolated as a white solid.

¹H NMR (MeOD, 300 MHz) δ 9.02-9.03 (d, J=1.92 Hz, 1H), 8.03-8.19 (m, 1H), 7.61-7.64 (dd, J=1.89, 6.39 Hz, 1H), 7.10-7.15 (m, 5H), 6.33-6.41 (dt, J=4.11, 2.4, 3.81, 14.88 Hz, 1H), 4.99-5.22 (m, 1H), 4.63-4.69 (m, 1H), 4.08-4.13 (m, 1H), 3.52-3.88 (m, 6H), 1.64-1.76 (m, 2H), 0.99-1.04 (t, J=7.47 Hz, 3H); LC/MS [M+H]=577.0.

### Example 25

### Synthesis of 4′-(2-carboxy-2-(((2R,3R,4S,5R)-5-(2-chloro-6-(propylamino)-9H-purin-9-yl)-4-fluoro-3-hydroxytetrahydrofuran-2-yl)methoxy)-2-(thiazol-4-yl)ethyl)-[1,1′-biphenyl]-2-carboxylic acid

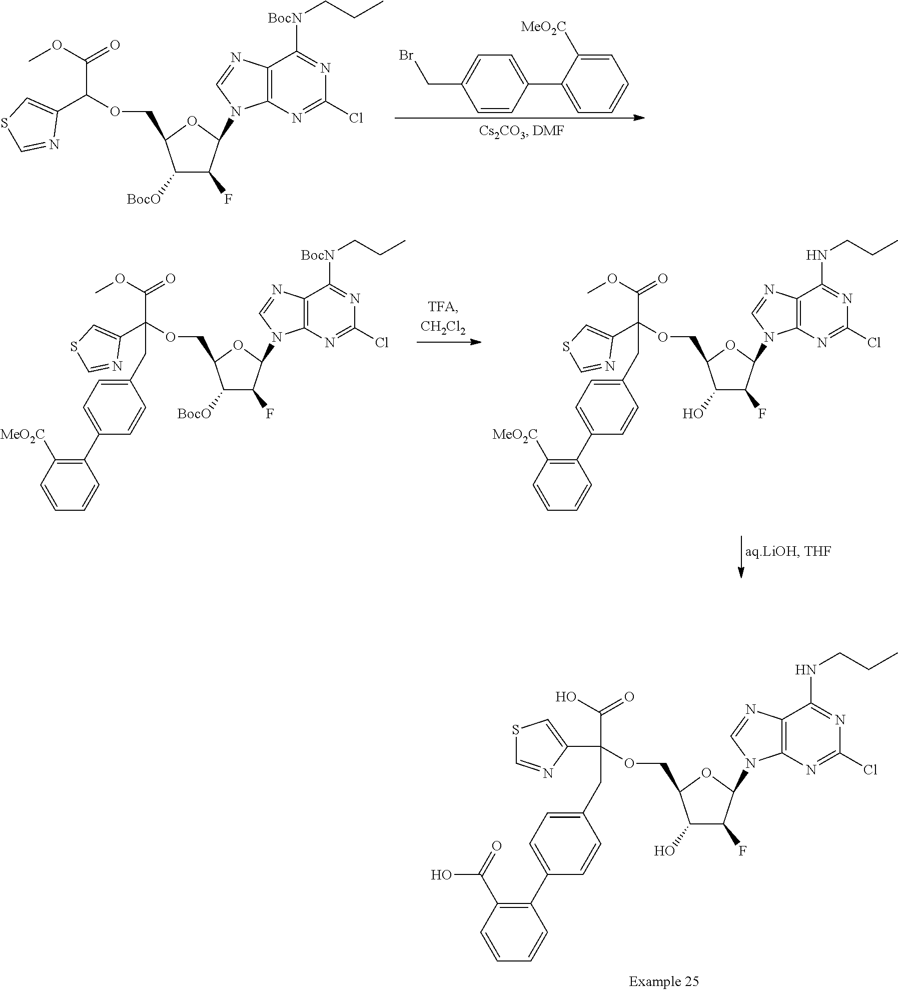

Proceeding as described in Example 1 above but substituting tert-butyl (9-((2R,3R,4R,5R)-3,4-bis((tert-butoxycarbonyl)oxy)-5-(hydroxymethyl)tetrahydrofuran-2-yl)-6-((tert-butoxycarbonyl)amino)-2-chloro-9H-purin-6-yl)carbamate and 4-(bromomethyl)-1,1′-biphenyl with methyl 2-(((2R,3R,4S,5R)-5-(6-((tert-butoxycarbonyl)-(propyl)amino)-2-chloro-9H-purin-9-yl)-3-((tert-butoxycarbonyl)oxy)-4-fluorotetrahydro-furan-2-yl)methoxy)-2-(thiazol-4-yl)acetate and methyl 4′-(bromomethyl)-[1,1′-biphenyl]-2-carboxylate provided the title compound as a mixture of diastereomers (ca. 1:1) and isolated as a white solid.

¹H NMR (MeOD, 300 MHz) δ 9.03 (s, 1H), 8.08-8.25 (d, J=51.09 Hz, 1H), 7.09-7.76 (m, 9H), 6.34-6.42 (dt, J=4.35, 3.66, 5.97, 15.48 Hz, 1H), 4.99-5.24 (m, 1H), 4.62-4.68 (m, 1H), 4.10-4.14 (m, 1H), 3.50-3.97 (m, 6H), 1.64-1.71 (m, 2H), 0.97-1.02 (t, J=7.23 Hz, 3H); LC/MS [M+H]=697.1.

### Example 26

### Synthesis of 2-(((2R,3S,4R,5R)-5-(6-amino-2-chloro-9H-purin-9-yl)-3,4-dihydroxytetrahydrofuran-2-yl)methoxy)-3-phenyl-2-(thiazol-4-yl)propanoic acid

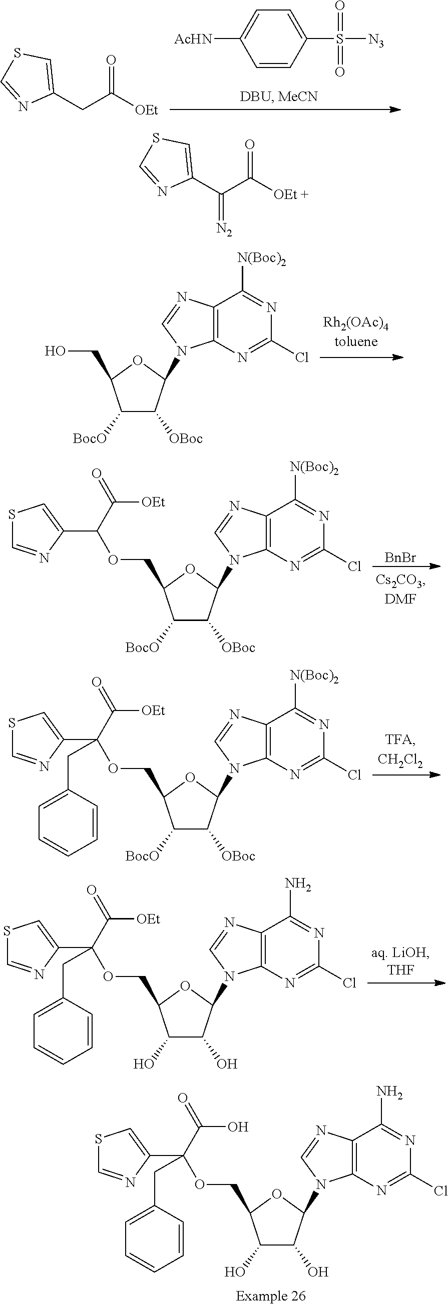

### Step 1:

To a solution of ethyl 2-(thiazol-4-yl)acetate (2.00 g, 11.7 mmole) in CH₃CN (15 mL) at 0° C. was added DBU (2.62 ml, 17.6 mmole) and 4-acetamidibenzene sulfonylazide (3.4 g, 14.1 mmole) in CH₃CN (10 mL). The reaction mixture was stirred at 25° C. for 1.5 h before it was concentrated under reduced pressure to dryness. The resulting crude was purified by silica gel column chromatography (0-40% EtOAc in hexanes) to provide ethyl 2-diazo-2-(thiazol-4-yl)acetate (2.0 g).

### Steps 2-5:

Proceeding as described in Example 1 above but substituting methyl 2-diazo-2-(thiazol-4-yl)acetate and 4-(bromomethyl)-1,1′-biphenyl with ethyl 2-diazo-2-(thiazol-4-yl)acetate and BnBr provided the title compound as a mixture of diastereomers (ca. 1:1) and isolated as a white solid.

¹H NMR (CD₃OD, 300 MHz): Isomer 1: δ 9.04-9.06 (m, 1H), 8.40 (s, 1H), 7.68-7.71 (m, 1H), 7.06-7.19 (m, 5H), 5.99 (d, J=5.76 Hz, 1H), 4.75 (t, J=5.31 Hz, 1H), 4.30-4.37 (m, 1H), 4.16-4.22 (m, 1H), 3.49-3.86 (m, 4H); Isomer 2: δ 9.02-9.04 (m, 1H), 8.29 (s, 1H), 7.64-7.67 (m, 1H), 7.06-7.19 (m, 5H), 5.95 (d, J=5.67 Hz, 1H), 4.69 (t, J=5.40 Hz, 1H), 4.16-4.22 (m, 1H), 4.30-4.37 (m, 1H), 3.49-3.86 (m, 4H); LC/MS [M+H]=533.2.

### Example 27

### Synthesis of 3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(2-chlorothiazol-4-yl)propanoic acid

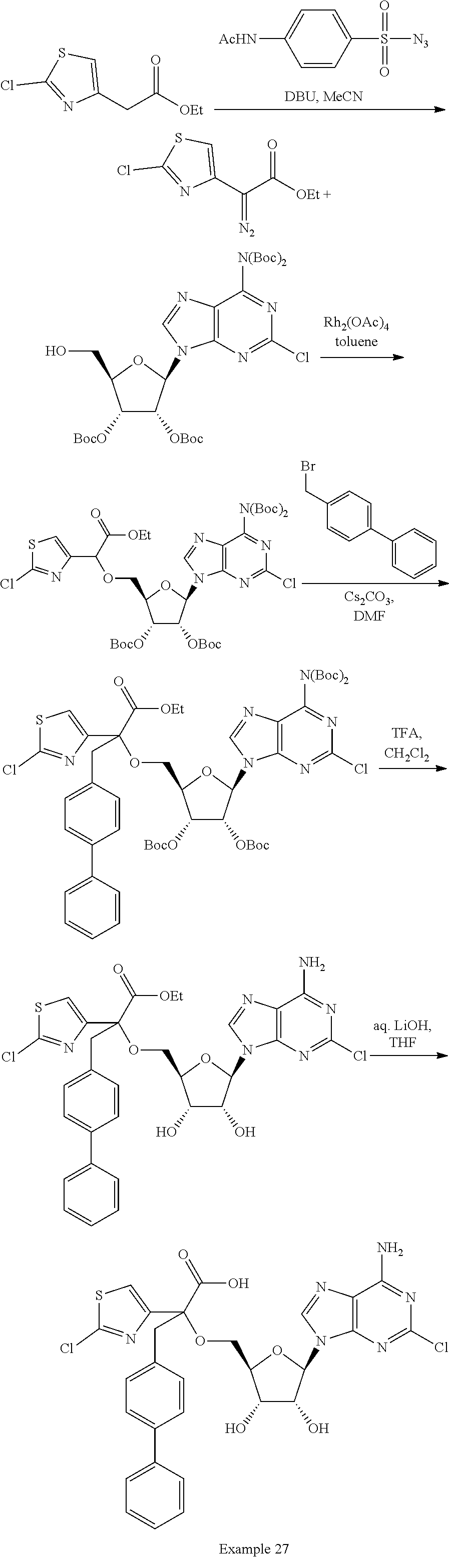

### Step 1:

To a solution of ethyl 2-(2-chlorothiazol-4-yl)acetate (2.40 g, 11.7 mmole) in CH₃CN (15 mL) at 0° C. was added DBU (2.62 ml, 17.6 mmole) and 4-acetamidibenzene sulfonylazide (3.4 g, 14.1 mmole) in CH₃CN (10 mL). The reaction mixture was stirred at 25° C. for 1.5 h before it was concentrated under reduced pressure to dryness. The resulting crude was purified by silica gel column chromatography (0-40% EtOAc in hexanes) to provide ethyl 2-diazo-2-(thiazol-4-yl)acetate (2.0 g).

### Steps 2-5:

Proceeding as described in Example 26 above but substituting ethyl 2-diazo-2-(thiazol-4-yl)acetate and 4-(bromomethyl)-1,1′-biphenyl with ethyl 2-(2-chlorothiazol-4-yl)-2-diazoacetate and 4-(bromomethyl)-1,1′-biphenyl provided the title compound as a mixture of diastereomers (ca. 1:1) and isolated as an off-white solid.

¹H NMR (CD₃OD, 300 MHz): Isomer 1: δ 8.46 (s, 1H), 7.60 (s, 1H), 7.18-7.51 (m, 9H), 5.96 (d, J=5.79 Hz, 1H), 4.75 (t, J=5.19 Hz, 1H), 4.35-4.39 (m, 1H), 4.20-4.24 (m, 1H), 3.85 (dd, J=10.17, 2.88 Hz, 1H), 3.55-3.75 (m, 3H); Isomer 2: δ 8.26 (s, 1H), 7.63 (s, 1H), 7.18-7.51 (m, 9H), 6.02 (d, J=5.52 Hz, 1H), 4.69 (t, J=5.30 Hz, 1H), 4.39-4.43 (m, 1H), 4.24-4.28 (m, 1H), 3.91 (dd, J=10.39, 2.85 Hz, 1H), 3.55-3.75 (m, 3H); LC/MS [M+H]=643.1.

### Examples 28 & 29

### Synthesis of (S)-3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(2-methylthiazol-4-yl)propanoic acid and (R)-3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(2-methylthiazol-4-yl)propanoic acid

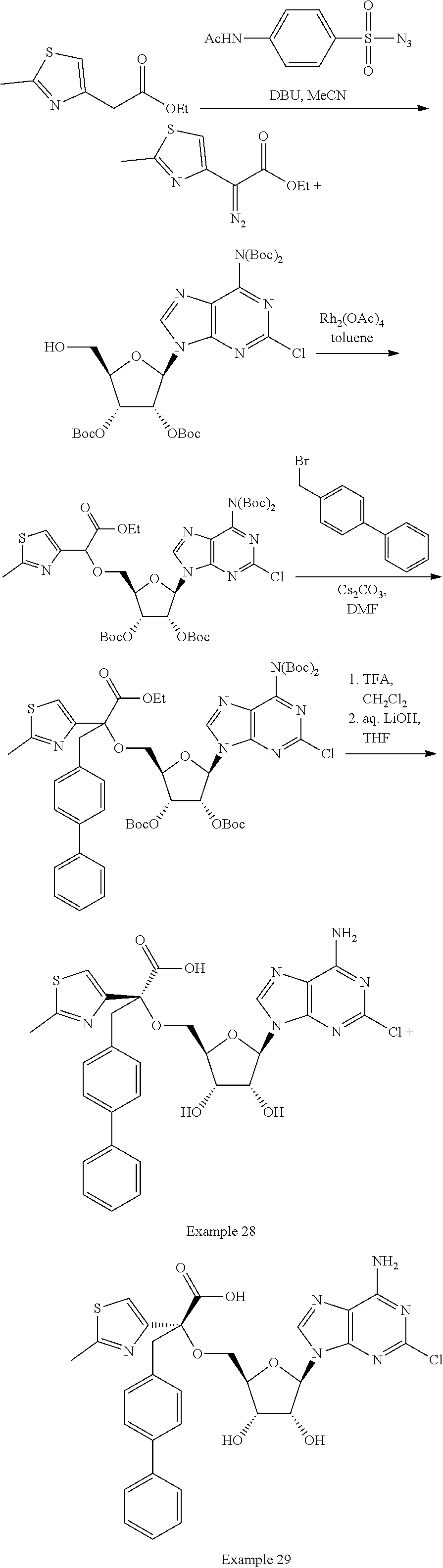

Proceeding as described in Example 1 above but substituting methyl 2-(thiazol-4-yl)acetate with ethyl 2-(2-methylthiazol-4-yl)acetate provided a pair of diastereomeric title products (ca. 1:1) which the stereo configuration was assigned arbitrarily. Both products were purified by preparative HPLC and isolated as white solids.

(S)-3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(2-methylthiazol-4-yl)propanoic acid: ¹H NMR (CD₃OD, 300 MHz) δ 8.29 (s, 1H), 7.22-7.53 (m, 10H), 5.95 (d, J=5.88 Hz, 1H), 4.68-4.73 (m, 1H), 4.36 (dd, J=4.83, 3.09 Hz, 1H), 4.18-4.23 (m, 1H), 3.79 (dd, J=10.24, 3.03 Hz, 1H), 3.76 (d, J=14.08 Hz, 1H), 3.64 (d, J=14.10 Hz, 1H), 3.55 (dd, J=10.19, 3.29 Hz, 1H), 4.73 (s, 3H); LC/MS [M+H]=623.2.

(R)-3-([1,1′-biphenyl]-4-yl)-2-(((2R,3S,4R,5R)-5-(6-amino-2-chloro-9H-purin-9-yl)-3,4-dihydroxytetrahydrofuran-2-yl)methoxy)-2-(2-methylthiazol-4-yl)propanoic acid: ¹H NMR (CD₃OD, 300 MHz) δ 8.49 (s, 1H), 7.52 (s, 1H), 7.22-7.49 (m, 9H), 6.01 (d, J=5.37 Hz, 1H), 4.73 (t, J=5.09 Hz, 1H), 4.37-4.42 (m, 1H), 4.21-4.26 (m, 1H), 3.92 (dd, J=10.40, 2.71 Hz, 1H), 3.78 (d, J=14.32 Hz, 1H), 3.65 (d, J=14.20 Hz, 1H), 3.53 (dd, J=10.38, 2.49 Hz, 1H), 2.75 (s, 3H); LC/MS [M+H]=623.2.

### Examples 30 & 31

### Synthesis of (S)-2-(((2R,3S,4R,5R)-5-(6-amino-2-chloro-9H-purin-9-yl)-3,4-dihydroxytetrahydrofuran-2-yl)methoxy)-2-(2-aminothiazol-4-yl)-3-phenylpropanoic acid and (R)-2-(((2R,3S,4R,5R)-5-(6-amino-2-chloro-9H-purin-9-yl)-3,4-dihydroxytetrahydrofuran-2-yl)methoxy)-2-(2-aminothiazol-4-yl)-3-phenylpropanoic acid

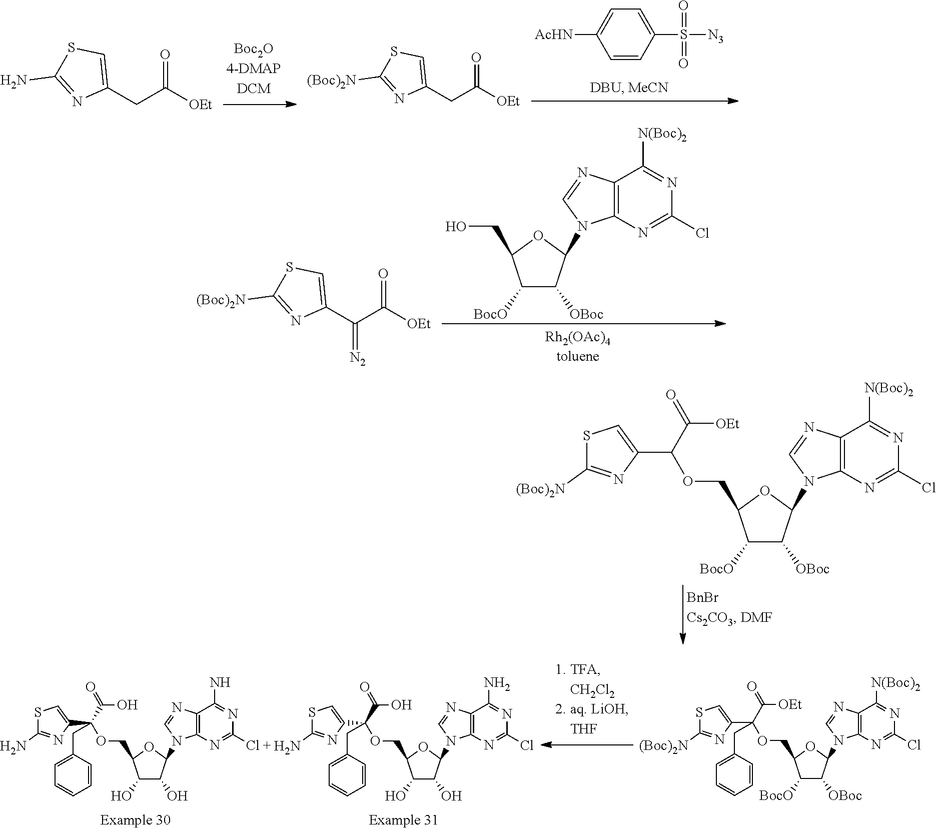

### Step 1:

To a solution of ethyl 2-(2-aminothiazol-4-yl) acetate (1.5 g, 8.06 mmol) in dry DCM (40 mL) at 25° C. under argon atmosphere was added 4-DMAP (110 mg, 0.9 mmol) and di-tert-butyl dicarbonate (4.574 g, 20.96 mmol). The reaction mixture was stirred overnight before it was concentrated. The crude residue was purified by CombiFlash chromatography on silica gel (10-68% EtOAc in hexanes) to provide ethyl 2-(2-N,N′-(bis-(tert-butoxy-carbonyl)amino)thiazol-4-yl)acetate (2.775 g) as a viscous oil.

### Steps 2-6:

Proceeding as described in Example 1 above but substituting methyl 2-(thiazol-4-yl)acetate with ethyl 2-(2-N,N′-(bis-(tert-butoxycarbonyl)amino)thiazol-4-yl)acetate provided a pair of diastereomeric title products (ca. 1:1) which the stereo configuration was assigned arbitrarily. These diastereomers also existed as a pair of tautomers. Both products were purified by preparative HPLC and isolated as off-white solids.

(S)-2-(((2R,3S,4R,5R)-5-(6-amino-2-chloro-9H-purin-9-yl)-3,4-dihydroxytetrahydrofuran-2-yl)methoxy)-2-(2-aminothiazol-4-yl)-3-phenylpropanoic acid: ¹H NMR (CD₃OD, 300 MHz): Tautomer 1: δ 8.33 (s, 1H), 7.10-7.31 (m, 5H), 6.59 (s, 1H), 5.93 (d, J=6.06 Hz, 1H), 4.66-4.71 (m, 1H), 4.30-4.37 (m, 2H), 3.70-3.81 (m, 1H), 3.54 (d, J=14.01 Hz, 1H), 3.46 (d, J=14.04 Hz, 1H), 3.07-3.13 (m, 1H); LC/MS [M+H]=548.1. Tautomer 2: δ 8.29 (s, 1H), 7.64 (s, 1H), 7.10-7.31 (m, 5H), 5.95 (d, J=5.94 Hz, 1H), 4.44-4.61 (m, 11H), 4.10-4.27 (m, 2H), 3.82-3.94 (m, 1H), 3.59-3.69 (m, 1H), 3.49-3.55 (m, 2H); LC/MS [M+H]=548.1.

(R)-2-(((2R,3S,4R,5R)-5-(6-amino-2-chloro-9H-purin-9-yl)-3,4-dihydroxytetrahydrofuran-2-yl)methoxy)-2-(2-aminothiazol-4-yl)-3-phenylpropanoic acid: ¹H NMR (CD₃OD, 300 MHz): Tautomer 1: δ 8.16 (s, 1H), 7.10-7.30 (m, 5H), 6.60 (s, 1H), 5.98 (d, J=5.67 Hz, 1H), 4.69-4.75 (m, 1H), 4.38-4.44 (m, 1H), 4.19-4.27 (m, 1H), 3.42-3.89 (m, 3H), 3.09-3.15 (m, 1H); LC/MS [M+H]=548.1. Tautomer 2: δ 8.27 (s, 1H), 7.56-7.61 (m, 1H), 7.10-7.30 (m, 5H), 6.60 (s, 1H), 5.95 (d, J=5.40 Hz, 1H), 4.53-4.60 (m, 1H), 4.44-4.51 (m, 1H), 4.30-4.37 (m, 1H), 3.42-3.89 (m, 3H), 3.09-3.15 (m, 1H); LC/MS [M+H]=548.1.

### Example 32

### Synthesis of 2-(((2R,3S,4R,5R)-5-(6-amino-2-chloro-9H-purin-9-yl)-3,4-dihydroxytetrahydrofuran-2-yl)methoxy)-3-phenyl-2-(1H-pyrazol-3-yl)propanoic acid

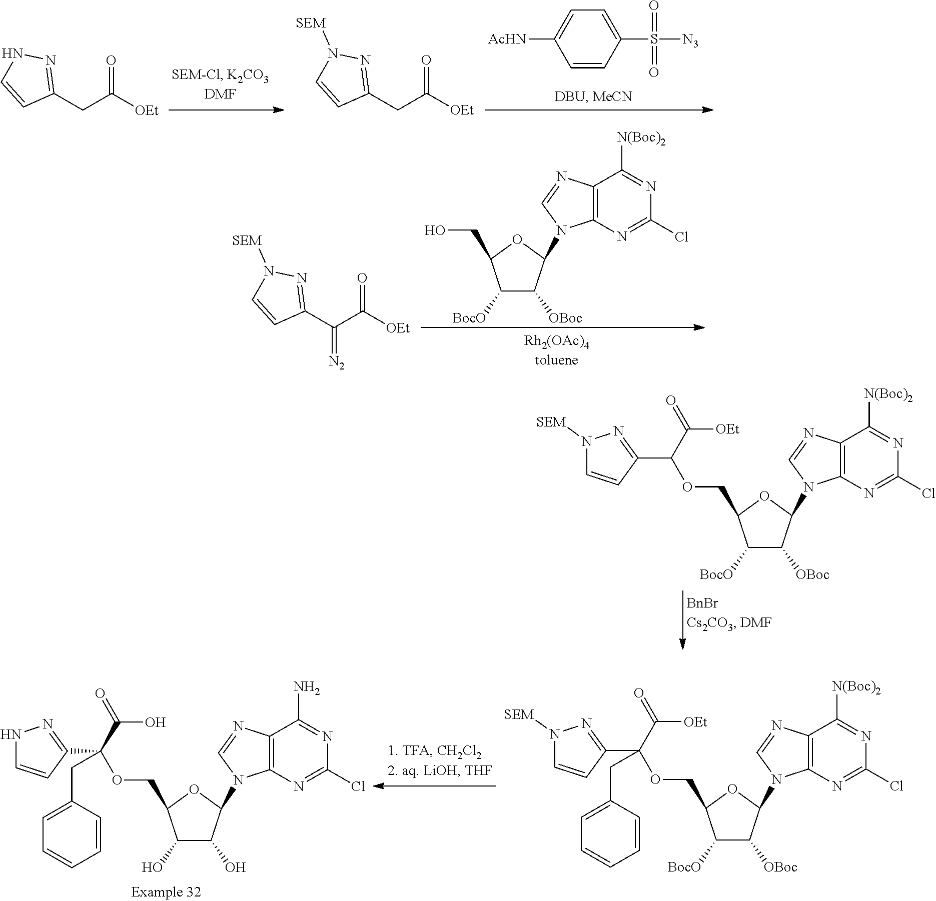

### Step 1:

To a solution of ethyl 2-(1H-pyrazol-3-yl)acetate (500 mg, 3.24 mmol) and trimethylsilyl)ethoxymethyl chloride (0.69 mL, 3.89 mmol) in dry DMF (7 mL) under argon atmosphere at 25° C. was added powdered potassium carbonate (896 mg, 6.48 mmol). The reaction mixture was stirred overnight before it was diluted with brine (30 mL) and EtOAc (30 mL). The organic layer was separated. The aqueous phase was extracted with EtOAc (2×30 mL). The combined organic layer was washed consecutively with brine (30 mL) and water (30 mL) and then dried over Na₂SO₄ and concentrated. The residue was purified by CombiFlash silica gel column chromatography (8-58% EtOAc in hexanes) to provide ethyl 2-(1-((2-(trimethylsilyl)ethoxy)methyl)-1H-pyrazol-3-yl)acetate (259 mg) as an oil.

### Steps 2-6:

Proceeding as described in Example 1 above but substituting methyl 2-(thiazol-4-yl)acetate with ethyl 2-(1-((2-(trimethylsilyl)ethoxy)methyl)-1H-pyrazol-3-yl)acetate provided the title compound as a mixture of diastereomers (ca. 1:1) and isolated as an off-white solid.

¹H NMR (CD₃OD, 300 MHz): Isomer 1: δ 8.40 (s, 1H), 7.68 (d, J=2.10 Hz, 1H), 7.07-7.29 (m, 5H), 6.43 (d, J=2.10 Hz, 1H), 6.01 (d, J=5.67 Hz, 1H), 4.77 (t, J=5.28 Hz, 1H), 4.14-4.33 (m, 2H), 3.47-3.90 (m, 4H); Isomer 2: δ 8.39 (s, 1H), 7.63 (d, J=2.16 Hz, 1H), 7.07-7.29 (m, 5H), 6.39 (d, J=2.13 Hz, 1H), 5.96 (d, J=5.97 Hz, 1H), 4.66 (t, J=5.10 Hz, 1H), 4.14-4.33 (m, 2H), 3.47-3.90 (m, 4H); LC/MS [M+H]=516.2.

### Examples 33 & 34

### Synthesis of (S)-2-(((2R,3S,4R,5R)-5-(6-amino-2-chloro-9H-purin-9-yl)-3,4-dihydroxytetrahydrofuran-2-yl)methoxy)-3-phenyl-2-(1H-1,2,4-triazol-3-yl)propanoic acid and (R)-2-(((2R,3S,4R,5R)-5-(6-amino-2-chloro-9H-purin-9-yl)-3,4-dihydroxytetrahydrofuran-2-yl)methoxy)-3-phenyl-2-(1H-1,2,4-triazol-3-yl)propanoic acid

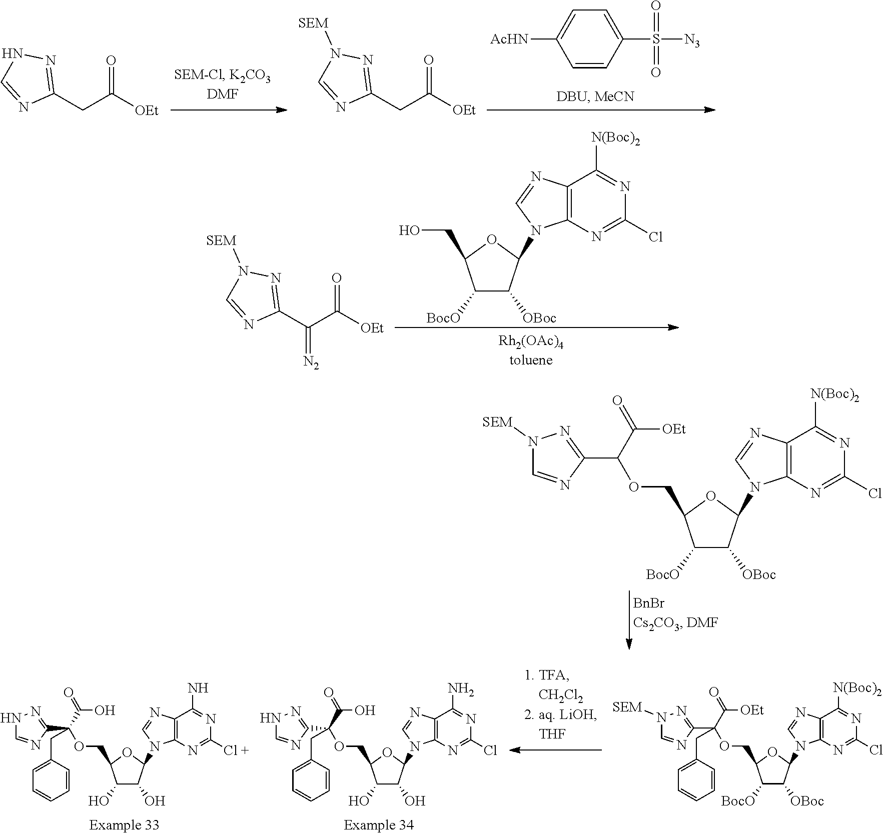

### Step 1:

To a solution of ethyl 2-(1H-1,2,4-triazol-3-yl)acetate (500 mg, 3.24 mmol) and trimethylsilyl)ethoxymethyl chloride (0.69 mL, 3.89 mmol) in dry DMF (7 mL) under argon atmosphere at 25° C. was added powdered potassium carbonate (896 mg, 6.48 mmol). The reaction mixture was stirred overnight before it was diluted with H₂O (30 mL) and extracted with EtOAc (3×30 mL). The combined organic layer was washed with brine (30 mL) and water (30 mL) and then dried over Na₂SO₄ and concentrated. The residue was purified by CombiFlash silica gel column chromatography (8-58% EtOAc in hexanes) to provide ethyl 2-(1-((2-(trimethylsilyl)ethoxy)methyl)-1H-1,2,4-triazol-3-yl)acetate (240 mg) as an oil.

### Steps 2-6:

Proceeding as described in Example 1 above but substituting methyl 2-(thiazol-4-yl)acetate with ethyl 2-(1-((2-(trimethylsilyl)ethoxy)methyl)-1H-1,2,4-triazol-3-yl)acetate provided a pair of diastereomeric title products (ca. 1:1) which the stereo configuration was assigned arbitrarily. Both products were purified by preparative HPLC and isolated as off-white solids.

(S)-2-(((2R,3S,4R,5R)-5-(6-amino-2-chloro-9H-purin-9-yl)-3,4-dihydroxytetrahydrofuran-2-yl)methoxy)-3-phenyl-2-(1H-1,2,4-triazol-3-yl)propanoic acid: ¹H NMR (CD₃OD, 300 MHz) δ 8.40 (s, 1H), 8.32 (s, 1H), 7.10-7.26 (m, 5H), 5.96 (d, J=5.91 Hz, 1H), 4.63-4.69 (m, 1H), 4.27-4.32 (m, 1H), 4.15-4.20 (m, 1H), 3.80 (d, J=14.20 Hz, 1H), 3.73-3.79 (m, 1H), 3.63 (d, J=14.2 Hz, 1H), 3.56 (dd, J=10.16, 3.29 Hz, 1H); LC/MS [M+H]=517.2.

(R)-2-(((2R,3S,4R,5R)-5-(6-amino-2-chloro-9H-purin-9-yl)-3,4-dihydroxytetrahydrofuran-2-yl)methoxy)-3-phenyl-2-(1H-1,2,4-triazol-3-yl)propanoic acid: ¹H NMR (CD₃OD, 300 MHz) δ 8.47 (s, 1H), 8.44 (s, 1H), 7.17-7.27 (m, 2H), 7.04-7.14 (m, 3H), 6.01 (d, J=5.67 Hz, 1H), 4.77 (t, J=5.27 Hz, 1H), 4.34-4.39 (m, 1H), 4.17-4.23 (m, 1H), 3.88-3.96 (m, 1H), 3.81 (d, J=14.71 Hz, 1H), 3.66 (d, J=14.70 Hz, 1H), 3.44-3.52 (m, 1H); LC/MS [M+H]=517.2.

### Examples 35 & 36

### Synthesis of (S)-2-(((2R,3S,4R,5R)-5-(6-amino-2-chloro-9H-purin-9-yl)-3,4-dihydroxytetrahydrofuran-2-yl)methoxy)-2-(oxazol-4-yl)-3-phenylpropanoic acid and (R)-2-(((2R,3S,4R,5R)-5-(6-amino-2-chloro-9H-purin-9-yl)-3,4-dihydroxytetrahydrofuran-2-yl)methoxy)-2-(oxazol-4-yl)-3-phenylpropanoic acid

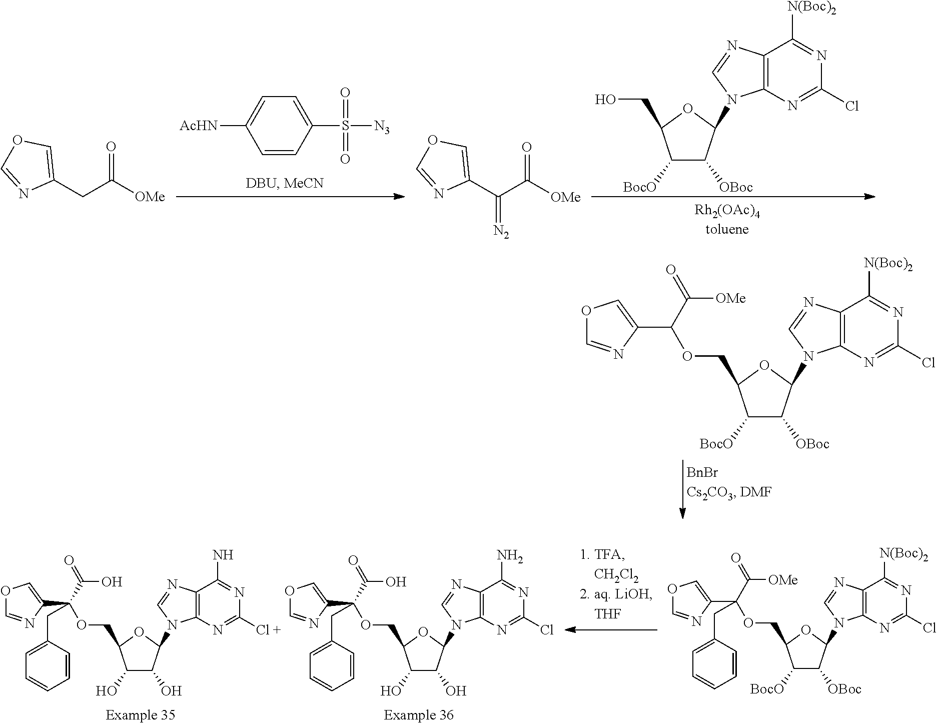

Proceeding as described in Example 1 above but substituting methyl 2-(thiazol-4-yl)acetate with methyl 2-(oxazol-4-yl)acetate provided a pair of diastereomeric title products (ca. 1:1) which the stereo configuration was assigned arbitrarily. Both products were purified by preparative HPLC and isolated as off-white solids.

(S)-2-(((2R,3S,4R,5R)-5-(6-amino-2-chloro-9H-purin-9-yl)-3,4-dihydroxytetrahydrofuran-2-yl)methoxy)-2-(oxazol-4-yl)-3-phenylpropanoic acid: ¹H NMR (CD₃OD, 300 MHz) δ 8.37 (s, 1H), 8.25 (d, J=0.70 Hz, 1H), 7.97 (d, J=0.71 Hz, 1H), 7.09-7.20 (m, 5H), 5.98 (d, J=5.60 Hz, 1H), 4.65 (t, J=5.31 Hz 1H), 4.37-4.41 (m, 1H), 4.16-4.21 (m, 1H), 3.72 (dd. J=10.21, 2.97 Hz, 1H), 3.58 (d, J=13.50 Hz, 1H), 3.51 (d, J=13.49 Hz, 1H), 3.48 (dd, J=10.13, 3.11 Hz, 1H); LC/MS [M+H]=517.2.

(R)-2-(((2R,3S,4R,5R)-5-(6-amino-2-chloro-9H-purin-9-yl)-3,4-dihydroxytetrahydrofuran-2-yl)methoxy)-2-(oxazol-4-yl)-3-phenylpropanoic acid: ¹H NMR (CD₃OD, 300 MHz) δ 8.55 (s, 1H), 8.26 (bs, 1H), 8.02 (bs, 1H), 7.11 (bs, 5H), 6.00 (d, J=5.43 Hz, 1H), 4.71 (t, J=5.13 Hz 1H), 4.30-4.44 (m, 1H), 4.20-4.24 (m, 1H), 3.75 (dd, J=10.24, 2.91 Hz, 1H), 3.48-3.62 (m, 3H); LC/MS [M+H]=517.2.

### Example 37

### Synthesis of 2-(((2R,3S,4R,5R)-5-(6-amino-2-chloro-9H-purin-9-yl)-3,4-dihydroxytetrahydrofuran-2-yl)methoxy)-2-(5-methylisoxazol-3-yl)-3-phenylpropanoic acid

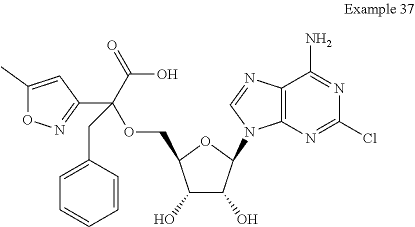 [SMILES: Cc1cc(C(Cc2ccccc2)(OC[C@H]2O[C@@H](n3cnc4c(N)nc(Cl)nc43)[C@H](O)[C@@H]2O)C(=O)O)no1]

Proceeding as described in Example 1 above but substituting methyl 2-(thiazol-4-yl)acetate with methyl 2-(5-methylisoxazol-3-yl)acetate provided the title compound as a mixture of diastereomers (ca. 1:1) and isolated as an off-white solid.

¹H NMR (CD₃OD, 300 MHz); Isomer 1: δ 8.25 (s, 1H), 7.14-7.22 (m, 5H), 6.46 (s, 1H), 5.95-6.01 (m, 1H), 4.66 (t, J=5.50 Hz, 1H), 4.28-4.32 (m, 1H), 4.18-4.25 (m, 1H), 3.76-3.89 (m, 1H), 3.50-3.67 (m, 3H), 2.28 (s, 3H); Isomer 2: δ 8.23 (s, 1H), 7.14-7.22 (m, 5H), 6.46 (s, 1H), 5.95-6.01 (m, 1H), 4.72 (t, J=5.50 Hz, 1H), 4.33-4.38 (m, 1H), 4.18-4.25 (m, 1H), 3.76-3.89 (m, 1H), 3.50-3.67 (m, 3H), 2.28 (s, 3H); LC/MS [M+H]=643.1.

### Example 38

### Synthesis of 2-(((2R,3S,4R,5R)-5-(6-amino-2-chloro-9H-purin-9-yl)-3,4-dihydroxytetrahydrofuran-2-yl)methoxy)-3-(2′-(N-methylsulfamoyl)-[1,1′-biphenyl]-4-yl)-2-(thiazol-4-yl)propanoic acid

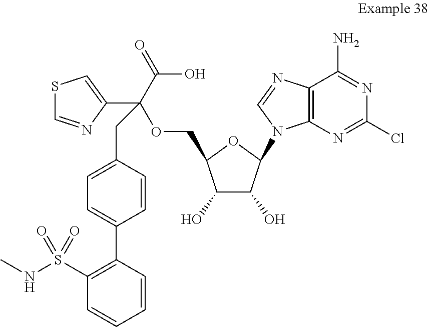 [SMILES: CNS(=O)(=O)c1ccccc1-c1ccc(CC(OC[C@H]2O[C@@H](n3cnc4c(N)nc(Cl)nc43)[C@H](O)[C@@H]2O)(C(=O)O)c2cscn2)cc1]

Proceeding as described in Examples 4 and 5 above but substituting 2-cyanomethylphenylboronic acid, pinacol ester with (2-(N-methylsulfamoyl)phenyl)boronic acid provided the title compound as a mixture of diasteromers (ca. 1:1) and isolated as an off-white solid.

¹H NMR (CD₃OD, 300 MHz); Isomer 1: δ 9.04-9.08 (m, 1H), 8.52 (s, 1H), 7.98-8.00 (m, 1H), 7.74 (d, J=1.98 Hz 1H), 7.49-7.63 (m, 2H), 7.15-7.29 (m, 5H), 5.99 (d, J=5.31 Hz, 1H), 4.73 (t, J=5.13 Hz, 11H), 4.34-4.41 (m, 1H), 4.17-4.24 (m, 1H), 3.83 (d, J=14.22 Hz, 1H), 3.72 (d, J=14.17 Hz, 1H), 3.64 (dd, J=10.25, 3.07 Hz, 1H), 3.55 (dd, J=10.10, 3.31 Hz, 1H), 2.28 (s, 3H). Isomer 2: δ 9.04-9.08 (m, 1H), 8.38 (s, 1H), 8.01-8.03 (m, 1H), 7.73 (d, J=1.95 Hz 1H), 7.49-7.63 (m, 2H), 7.15-7.29 (m, 5H), 5.96 (d, J=5.58 Hz, 1H), 4.66 (t, J=5.22 Hz, 1H), 4.34-4.41 (m, 1H), 4.17-4.24 (m, 1H), 3.77-3.87 (m, 2H), 3.53-3.64 (m, 2H), 2.29 (s, 3H), LC/MS [M+H]=702.2.

### Examples 39 & 40

### Synthesis of (S)-2-(((2R,3S,4R,5R)-5-(6-amino-2-chloro-9H-purin-9-yl)-3,4-dihydroxytetrahydrofuran-2-yl)methoxy)-3-(2′-(hydroxymethyl)-6′-methoxy-[1,1′-biphenyl]-4-yl)-2-(thiazol-4-yl)propanoic acid and (R)-2-(((2R,3S,4R,5R)-5-(6-amino-2-chloro-9H-purin-9-yl)-3,4-dihydroxytetrahydrofuran-2-yl)methoxy)-3-(2′-(hydroxymethyl)-6′-methoxy-[1,1′-biphenyl]-4-yl)-2-(thiazol-4-yl)propanoic acid

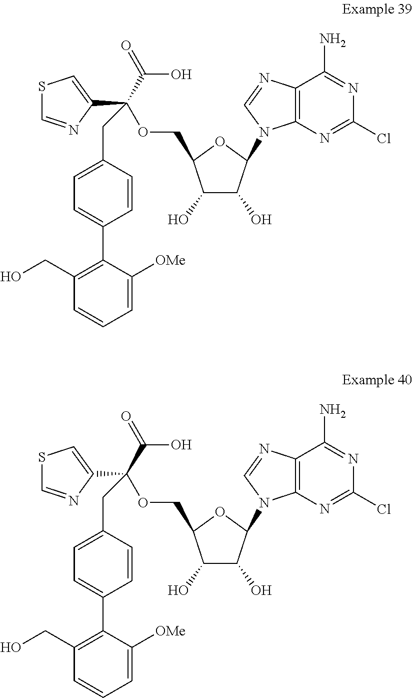 [SMILES: COc1cccc(CO)c1-c1ccc(C[C@@](OC[C@H]2O[C@@H](n3cnc4c(N)nc(Cl)nc43)[C@H](O)[C@@H]2O)(C(=O)O)c2cscn2)cc1 ; COc1cccc(CO)c1-c1ccc(C[C@](OC[C@H]2O[C@@H](n3cnc4c(N)nc(Cl)nc43)[C@H](O)[C@@H]2O)(C(=O)O)c2cscn2)cc1]

Proceeding as described in Examples 4 and 5 above but substituting 2-cyanomethylphenylboronic acid, pinacol ester with (2-(hydroxymethyl)-6-methoxyphenyl)boronic acid provided a pair of diastereomeric title products (ca. 1:1) which the stereo configuration was assigned arbitrarily. Both products were purified by preparative HPLC and isolated as off-white solids.

(S)-2-(((2R,3S,4R,5R)-5-(6-amino-2-chloro-9H-purin-9-yl)-3,4-dihydroxytetrahydrofuran-2-yl)methoxy)-3-(2′-(hydroxymethyl)-6′-methoxy-[1,1′-biphenyl]-4-yl)-2-(thiazol-4-yl)propanoic acid: ¹H NMR (CD₃OD, 300 MHz) δ 9.03 (d, J=1.92 Hz 1H), 8.47 (s, 1H), 7.68 (d, J=1.95 Hz, 1H), 7.11-7.34 (m, 4H), 7.03 (d, J=8.38 Hz, 2H), 6.92 (d, J=8.14 Hz, 1H), 5.98 (d, J=5.91 Hz, 1H), 4.73 (t, J=5.34 Hz, 1H), 4.29-4.33 (m, 1H), 4.18-4.24 (m, 3H), 3.81 (d, J=14.14 Hz, 1H), 3.73 (d, J=14.3 Hz, 1H), 3.71-3.78 (m, 1H), 3.55-3.63 (m, 1H), 3.60 (s, 3H); LC/MS [M+H]=669.2.

(R)-2-(((2R,3S,4R,5R)-5-(6-amino-2-chloro-9H-purin-9-yl)-3,4-dihydroxytetrahydrofuran-2-yl)methoxy)-3-(2′-(hydroxymethyl)-6′-methoxy-[1,1′-biphenyl]-4-yl)-2-(thiazol-4-yl)propanoic acid: ¹H NMR (CD₃OD, 300 MHz) δ 9.05 (d, J=1.83 Hz 1H), 8.54 (s, 1H), 7.72 (d, J=1.86 Hz, 1H), 7.27-7.34 (m, 1H), 7.13-7.24 (m, 3H), 6.88-7.03 (m, 3H), 6.01 (d, J=5.91 Hz, 1H), 4.81 (t, J=5.42 Hz, 1H), 4.33-4.37 (m, 1H), 4.32 (d, J=13.01 Hz, 1H), 4.24 (d, J=13.18 Hz, 1H), 4.19-4.24 (m, 1H), 3.82-3.88 (m, 1H), 3.81 (d, J=14.16 Hz, 1H), 3.69 (d, J=14.02 Hz, 1H), 3.60 (s, 3H), 3.57-3.63 (m, 1H); LC/MS [M+H]=669.2.

### Examples 41 & 42

### Synthesis of (S)-2-(((2R,3S,4R,5R)-5-(6-amino-2-chloro-9H-purin-9-yl)-3,4-dihydroxytetrahydrofuran-2-yl)methoxy)-3-(2′-(methylsulfonamido)-[1,1′-biphenyl]-4-yl)-2-(thiazol-4-yl)propanoic acid and (R)-2-(((2R,3S,4R,5R)-5-(6-amino-2-chloro-9H-purin-9-yl)-3,4-dihydroxytetrahydrofuran-2-yl)methoxy)-3-(2′-(methylsulfonamido)-[1,1′-biphenyl]-4-yl)-2-(thiazol-4-yl)propanoic acid

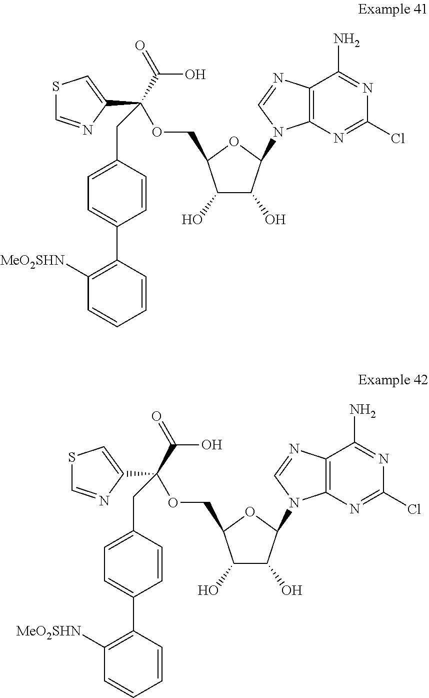

Proceeding as described in Examples 4 and 5 above but substituting 2-cyanomethylphenylboronic acid, pinacol ester with (2-(methylsulfonamido)phenyl)boronic acid provided a pair of diastereomeric title products (ca. 1:1) which the stereo configuration was assigned arbitrarily. Both products were purified by preparative HPLC and isolated as off-white solids.

(S)-2-(((2R,3S,4R,5R)-5-(6-amino-2-chloro-9H-purin-9-yl)-3,4-dihydroxytetrahydrofuran-2-yl)methoxy)-3-(2′-(methylsulfonamido)-[1,1′-biphenyl]-4-yl)-2-(thiazol-4-yl)propanoic acid: ¹H NMR (CD₃OD, 300 MHz) δ 9.06 (d, J=1.95 Hz 11H), 8.18 (s, 1H), 7.69 (d, J=1.95 Hz, 1H), 7.49 (dd, J=7.99, 1.32 Hz, 1H), 7.17-7.40 (m, 7H), 5.96 (d, J=5.58 Hz, 1H), 4.68 (t, J=5.27 Hz, 1H), 4.37-4.42 (m, 1H), 4.18-4.23 (m, 1H), 3.86 (dd, J=10.16, 2.96 Hz, 1H), 3.83 (d, J=14.14 Hz, 1H), 3.69 (d, J=14.08 Hz, 1H), 3.53 (dd, J=10.28, 2.80 Hz, 1H), 2.70 (s, 3H); LC/MS [M+H]=702.2.

(R)-2-(((2R,3S,4R,5R)-5-(6-amino-2-chloro-9H-purin-9-yl)-3,4-dihydroxytetrahydrofuran-2-yl)methoxy)-3-(2′-(methylsulfonamido)-[1,1′-biphenyl]-4-yl)-2-(thiazol-4-yl)propanoic acid: ¹H NMR (CD₃OD, 300 MHz) δ9.07 (d, J=1.45 Hz 1H), 8.39 (s, 1H), 7.77 (d, J=1.46 Hz, 1H), 7.50 (dd, J=7.88, 1.32 Hz, 1H), 7.17-7.39 (m, 7H), 6.01 (d, J=5.97 Hz, 1H), 4.80-4.84 (m, 1H), 4.34-4.39 (m, 1H), 4.19-4.24 (m, 1H), 3.85-3.92 (m, 1H), 3.71 (d, J=14.64 Hz, 1H), 3.84 (d, J=14.53 Hz, 1H), 3.48-3.55 (m, 1H), 2.72 (s, 3H); LC/MS [M+H]=702.2

### Examples 43 & 44

### Synthesis of (S)-2-(((2R,3S,4R,5R)-5-(6-amino-2-chloro-9H-purin-9-yl)-3,4-dihydroxytetrahydrofuran-2-yl)methoxy)-2-(thiazol-4-yl)-3-(2′-(trifluoromethoxy)-[1,1′-biphenyl]-4-yl)propanoic acid and (R)-2-(((2R,3S,4R,5R)-5-(6-amino-2-chloro-9H-purin-9-yl)-3,4-dihydroxytetrahydrofuran-2-yl)methoxy)-2-(thiazol-4-yl)-3-(2′-(trifluoromethoxy)-[1,1′-biphenyl]-4-yl)propanoic acid

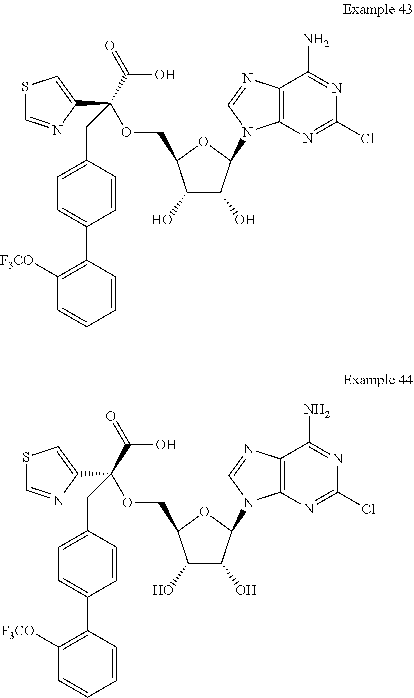 [SMILES: Nc1nc(Cl)nc2c1ncn2[C@@H]1O[C@H](CO[C@](Cc2ccc(-c3ccccc3OC(F)(F)F)cc2)(C(=O)O)c2cscn2)[C@@H](O)[C@H]1O ; Nc1nc(Cl)nc2c1ncn2[C@@H]1O[C@H](CO[C@@](Cc2ccc(-c3ccccc3OC(F)(F)F)cc2)(C(=O)O)c2cscn2)[C@@H](O)[C@H]1O]

Proceeding as described in Examples 4 and 5 above but substituting 2-cyanomethylphenylboronic acid, pinacol ester with (2-(trifluoromethoxy)phenyl)boronic acid provided a pair of diastereomeric title products (ca. 1:1) which the stereo configuration was assigned arbitrarily. Both products were purified by preparative HPLC and isolated as off-white solids.

(S)-2-(((2R,3S,4R,5R)-5-(6-amino-2-chloro-9H-purin-9-yl)-3,4-dihydroxytetrahydrofuran-2-yl)methoxy)-2-(thiazol-4-yl)-3-(2′-(trifluoromethoxy)-[1,1′-biphenyl]-4-yl)propanoic acid: ¹H NMR (CD₃OD, 300 MHz) δ 9.07 (s, 1H), 8.45 (s, 1H), 7.72 (s, 1H), 7.35-7.37 (m, 4H), 7.21-7.27 (m, 4H), 6.00-6.02 (d, J=5 Hz, 1H), 4.76 (bs, 1H), 4.40 (s, 1H), 4.23 (s, 1H), 3.72-3.89 (m, 3H), 3.60-3.64 (m, 1H); LC/MS [M+H]=693.1.

(R)-2-(((2R,3S,4R,5R)-5-(6-amino-2-chloro-9H-purin-9-yl)-3,4-dihydroxytetrahydrofuran-2-yl)methoxy)-2-(thiazol-4-yl)-3-(2′-(trifluoromethoxy)-[1,1′-biphenyl]-4-yl)propanoic acid: ¹H NMR (CD₃OD, 300 MHz) δ 9.06 (s, 1H), 8.25 (s, 1H), 7.68 (s, 1H), 7.32-7.37 (m, 4H), 7.26 (bs, 4H), 5.95-5.97 (d, J=6 Hz, 1H), 4.67 (bs, 1H), 4.40 (s, 1H), 4.21 (s, 1H), 3.70-3.89 (m, 3H), 3.56 (bs, 1H); LC/MS [M+H]=693.1.

### Example 45

### Synthesis of 2-(((2R,3S,4R,5R)-5-(6-amino-2-chloro-9H-purin-9-yl)-3,4-dihydroxytetrahydrofuran-2-yl)methoxy)-3-(2′-(diethylcarbamoyl)-[1,1′-biphenyl]-4-yl)-2-(thiazol-4-yl)propanoic acid

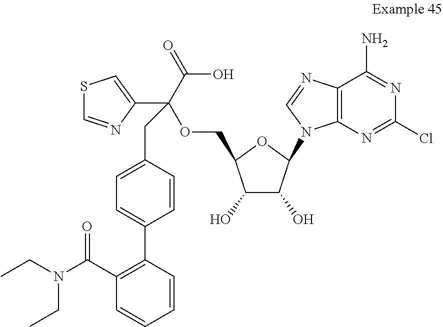 [SMILES: CCN(CC)C(=O)c1ccccc1-c1ccc(CC(OC[C@H]2O[C@@H](n3cnc4c(N)nc(Cl)nc43)[C@H](O)[C@@H]2O)(C(=O)O)c2cscn2)cc1]

Proceeding as described in Examples 4 and 5 above but substituting 2-cyanomethylphenylboronic acid, pinacol ester with (2-(diethylcarbamoyl)phenyl)boronic acid provided the title compound as a mixture of diastereomers (ca. 1:1) and isolated as a white solid.

¹H NMR (CD₃OD, 300 MHz) δ 9.07 (s, 1H), 8.53-8.57 (d, J=11 Hz, 1H), 8.09 (s, 1H), 7.74 (bs, 1H), 7.20-7.47 (m, 8H), 6.00-6.01 (m, 1H), 4.69-4.76 (m, 1H), 4.37 (s, 1H), 4.22 (s, 1H), 3.69-3.84 (m, 3H), 3.57-3.60 (m, 2H), 3.05-3.09 (m, 2H), 2.56-2.74 (m, 1H), 0.93 (bs, 3H), 0.71 (bs, 3H); LC/MS [M+H]=708.3.

### Examples 46, 47 & 48

### Synthesis of 2-(((2R,3S,4R,5R)-5-(6-amino-2-chloro-9H-purin-9-yl)-3,4-dihydroxytetrahydrofuran-2-yl)methoxy)-3-(2′-(methoxycarbonyl)-[1,1′-biphenyl]-4-yl)-2-phenylpropanoic acid and 4′-((S)-2-(((2R,3S,4R,5R)-5-(6-amino-2-chloro-9H-purin-9-yl)-3,4-dihydroxytetrahydrofuran-2-yl)methoxy)-2-carboxy-2-phenylethyl)-[1,1′-biphenyl]-2-carboxylic acid and 4′-((R)-2-(((2R,3S,4R,5R)-5-(6-amino-2-chloro-9H-purin-9-yl)-3,4-dihydroxytetrahydrofuran-2-yl)methoxy)-2-carboxy-2-phenylethyl)-[1,1′-biphenyl]-2-carboxylic acid

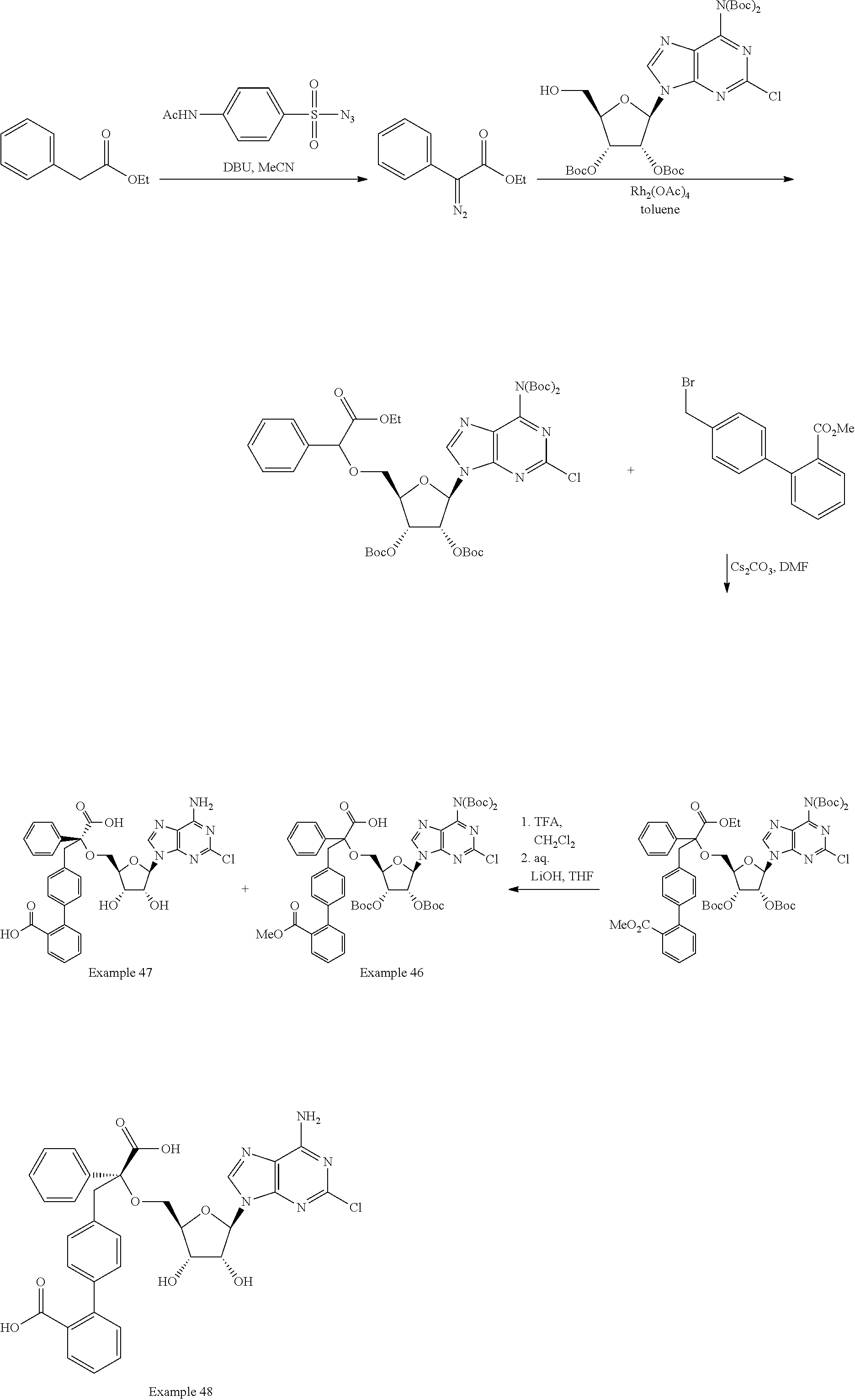

Proceeding as described in Example 1 above but substituting methyl 2-(thiazol-4-yl)acetate with ethyl 2-phenylacetate provided title compounds as 2-(((2R,3S,4R,5R)-5-(6-amino-2-chloro-9H-purin-9-yl)-3,4-dihydroxytetrahydrofuran-2-yl)methoxy)-3-(2′-(methoxy-carbonyl)-[1,1′-biphenyl]-4-yl)-2-phenylpropanoic acid and a pair of diastereomeric title products (ca. 1:1) which the stereo configuration was assigned arbitrarily: 4′-((S)-2-(((2R,3S,4R,5R)-5-(6-amino-2-chloro-9H-purin-9-yl)-3,4-dihydroxytetrahydrofuran-2-yl)methoxy)-2-carboxy-2-phenylethyl)-[1,1′-biphenyl]-2-carboxylic acid and 4′-((R)-2-(((2R,3S,4R,5R)-5-(6-amino-2-chloro-9H-purin-9-yl)-3,4-dihydroxytetrahydrofuran-2-yl)methoxy)-2-carboxy-2-phenylethyl)-[1,1′-biphenyl]-2-carboxylic acid. All title products were purified by preparative HPLC and isolated as white solids.

2-(((2R,3S,4R,5R)-5-(6-amino-2-chloro-9H-purin-9-yl)-3,4-dihydroxytetrahydrofuran-2-yl)methoxy)-3-(2′-(methoxy-carbonyl)-[1,1′-biphenyl]-4-yl)-2-phenylpropanoic acid: ¹H NMR (CD₃OD, 300 MHz) δ 8.51 (bs, 1H), 7.73 (d, J=7.6 Hz, 1H), 7.51-7.42 (m, 3H), 7.39-7.30, (m, 5H), 7.25-7.22 (m, 4H), 5.98 (d, J=5.6 Hz, 1H), 4.68 (t, 0.1=6.0 Hz, 1H), 4.18-4.15 (m, 2H), 3.86-3.67 (m, 6H), 3.58-3.53 (m, 1H); LC/MS [M+H]=660.1.

4′-((S)-2-(((2R,3S,4R,5R)-5-(6-amino-2-chloro-9H-purin-9-yl)-3,4-dihydroxytetrahydrofuran-2-yl)methoxy)-2-carboxy-2-phenylethyl)-[1,1′-biphenyl]-2-carboxylic acid: ¹H NMR (CD₃OD, 300 MHz) δ 8.60 (bs, 1H), 7.73 (d, J=7.0 Hz, 1H), 7.57-7.20 (m, 12H), 5.99 (d. J=6.1 Hz, 1H), 4.71 (t, J=5.8 Hz, 1H), 4.17 (d, J=2.2 Hz, 1H), 4.10-4.08 (m, 1H), 3.93-3.56 (m, 4H); LC/MS [M+H]=646.2.

4′-((R)-2-(((2R,3S,4R,5R)-5-(6-amino-2-chloro-9H-purin-9-yl)-3,4-dihydroxytetrahydrofuran-2-yl)methoxy)-2-carboxy-2-phenylethyl)-[1,1′-biphenyl]-2-carboxylic acid: ¹H NMR (CD₃OD, 300 MHz) δ 8.52 (bs, 1H), 7.76 (d, J=6.6 Hz, 1H), 7.59-7.11 (m, 12H), 6.06 (bs, 1H), 4.70 (1H, overlapping with water peak), 4.21 (bs, 2H), 3.95 (d, J=9.9 Hz, 1H), 3.73 (bs, 2H), 3.43 (d, J=10.8 Hz, 1H); LC/MS [M+H]=646.2

### Example 49

### Synthesis of 4′-(2-(((2R,3S,4R,5R)-5-(6-amino-2-chloro-9H-purin-9-yl)-3,4-dihydroxytetrahydrofuran-2-yl)methoxy)-2-carboxy-2-(thiophen-3-yl)ethyl)-[1,1′-biphenyl]-2-carboxylic acid

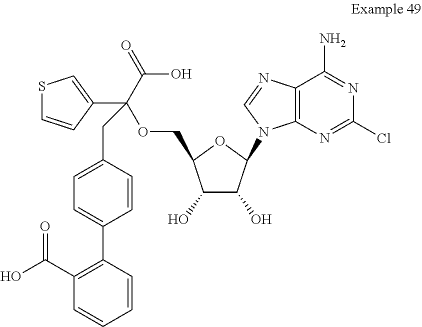 [SMILES: Nc1nc(Cl)nc2c1ncn2[C@@H]1O[C@H](COC(Cc2ccc(-c3ccccc3C(=O)O)cc2)(C(=O)O)c2ccsc2)[C@@H](O)[C@H]1O]

Proceeding as described in Example 1 above but substituting methyl 2-(thiazol-4-yl)acetate with ethyl 2-(thiophen-3-yl)acetate provided the title compound as a mixture of diastereomers (ca. 1:1) and isolated as a white solid.

¹H NMR (CD₃OD, 300 MHz) δ 8.52 (s, 1H), 7.75-7.77 (d, J=7.29 Hz, 1H), 7.16-7.53 (m, 10H), 6.00 (s, 1H), 4.65-4.68 (d, J=8.67 Hz, 1H), 4.17-4.23 (m, 2H), 3.54-3.80 (m, 4H); LC/MS [M+H]=652.0.

### Example 50

### Synthesis of 4′-(2-(((2S,4R,5R)-5-(6-amino-2-chloro-9H-purin-9-yl)-4-hydroxytetrahydrofuran-2-yl)methoxy)-2-carboxy-2-(thiazol-4-yl)ethyl)-[1,1′-biphenyl]-2-carboxylic acid

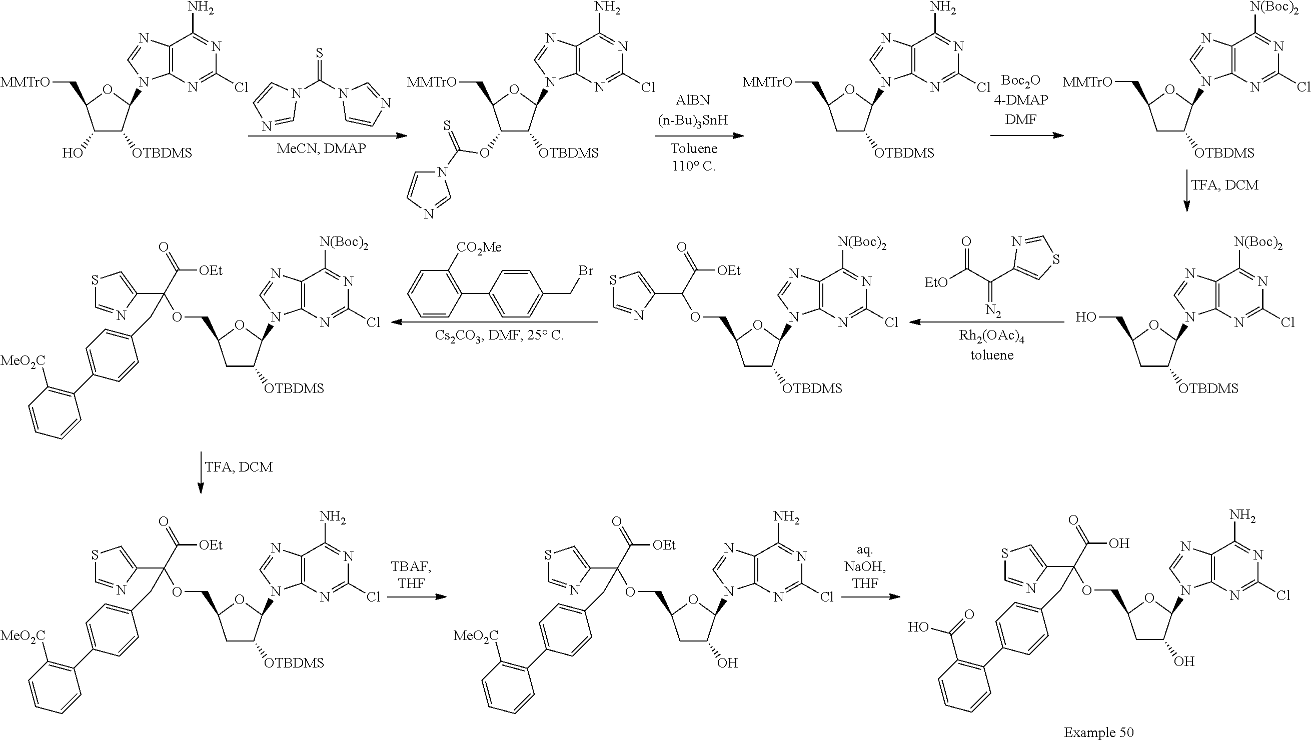

### Step 1.

To a solution of (2R,3R,4R,5R)-5-(6-amino-2-chloro-9H-purin-9-yl)-4-((tert-butyl-dimethylsilyl)oxy)-2-(((4-methoxyphenyl)diphenylmethoxy)methyl)tetrahydrofuran-3-ol (1.41 g, 2.05 mmol, 1 eq) in acetonitrile (35 mL) was added di-(imidazol-1-yl)methanethione (876 mg, 4.92 mmol, 2.4 eq). The resulting mixture was warmed to 70° C. and stirred for 5 h before it was concentrated to dryness. The residue was purified by flash column chromatography ion SiO₂ (40% EtOAc in petroleum ether) to provide 0-(((4-methoxyphenyl)-diphenylmethoxy)methyl) tetrahydrofuran-3-yl) 1H-imidazole-1-carbothioate (1.16 g, 71% yield) as a white solid.

### Step 2:

To a solution of 0-((2R,3R,4R,5R)-5-(6-amino-2-chloro-9H-purin-9-yl)-4-((tert-butyldimethylsilyl)oxy)-2-(((4-methoxyphenyl)diphenylmethoxy)methyl) tetrahydrofuran-3-yl) 1H-imidazole-1-carbothioate (1.16 g, 1.45 mmol, 1 eq.) in toluene (17 mL) was added AIBN (48 mg, 0.29 mmol, 0.2 eq.) under argon atmosphere. The reaction was heated at 110° C. and (n-Bu)₃SnH (468 μL, 1.74 mmol, 1.2 eq.) was added to the reaction carefully dropwise. The reaction was stirred at 110° C. for 1 h before it was quenched with sat. KF aq. (3 mL) and the reaction mixture was concentrated to dryness. The reaction was purified by flash column chromatography on SiO₂ (30% EtOAc in petroleum ether) to provide 9-((2R,3R,5S)-3-((tert-butyldimethylsilyl)oxy)-5-(((4-methoxyphenyl)diphenylmethoxy)-methyl)tetrahydrofuran-2-yl)-2-chloro-9H-purin-6-amine (610 mg, 63% yield) as a white solid.

### Step 3:

To a solution of 9-((2R,3R,5S)-3-((tert-butyldimethylsilyl)oxy)-5-(((4-methoxy-phenyl)diphenylmethoxy)methyl) tetrahydrofuran-2-yl)-2-chloro-9H-purin-6-amine (610 mg, 0.907 mmol, 1 eq.) in DMF (1.2 mL) was added 4-DMAP (28 mg, 0.227 mmol, 0.25 eq.) and Boc₂O (594 mg, 2.72 mmol, 3.0 eq.). The resulting mixture was stirred at 25° C. for 2 h before it was diluted with H₂O (50 mL), extracted with EtOAc (4×20 mL). The combined organic layers were washed with brine (2×30 mL), dried over magnesium sulfate, filtered and concentrated to dryness to provide crude tert-butyl (9-((2R,3R,5S)-3-((tert-butyldimethyl-silyl)oxy)-5-(((4-methoxyphenyl)diphenylmethoxy)methyl)tetrahydrofuran-2-yl)-6-((tert-butoxycarbonyl)amino)-2-chloro-9H-purin-6-yl)carbamate which was used in the next step without further purification.

### Step 4:

To a solution of the crude product from above in DCM (15 mL) was added a solution of TFA (337 μL, 4.54 mmol, 5.0 eq.) in DCM (15 mL) at 0° C. dropwise. The resulting mixture was stirred at 25° C. for 6 h before it was quenched with TEA (2 mL) and concentrated to dryness. The residue was purified by flash column chromatography on SiO₂ (20% EtOAc in petroleum ether) to provide tert-butyl (9-((2R,3R,5S)-3-((tert-butyldimethyl-silyl)oxy)-5-(hydroxymethyl)tetrahydrofuran-2-yl)-6-((tert-butoxycarbonyl)amino)-2-chloro-9H-purin-6-yl)carbamate (395 mg, 73% yield for 2 steps) as a white solid.

### Step 5:

To a solution of tert-butyl (9-((2R,3R,5S)-3-((tert-butyldimethyl-silyl)oxy)-5-(hydroxylmethyl)tetrahydrofuran-2-yl)-6-((tert-butoxycarbonyl)amino)-2-chloro-9H-purin-6-yl)carbamate (395 mg, 0.658 mmol, 1 eq.) and Rh₂(OAc)₄ (58 mg, 0.132 mmol, 0.2 eq.) in toluene (4 mL) was added a solution of ethyl 2-diazo-3-oxo-3-(thiazol-4-yl)propanoate (145 mg, 0.788 mmol, 1.2 eq.) in toluene (1 mL) dropwise at 95° C. under N₂ atmosphere. The resulting mixture was stirred at 95° C. for 8 h before it was concentrated to dryness. The residue was purified by flash column chromatography on SiO₂ (20% EtOAc in petroleum ether) to provide ethyl 2-(((2S,4R,5R)-5-(6-(N,N′-bis-(tert-butoxycarbonyl)amino)-2-chloro-9H-purin-9-yl)-4-((tert-butyldimethylsilyl)oxy)tetrahydrofuran-2-yl)methoxy)-2-(thiazol-4-yl)acetate (236 mg, 54% yield) as a light yellow gum.

### Step 6:

To a solution of ethyl 2-(((2S,4R,5R)-5-(6-(N,N′-bis-(tert-butoxycarbonyl)amino)-2-chloro-9H-purin-9-yl)-4-((tert-butyldimethylsilyl)oxy)tetrahydrofuran-2-yl)methoxy)-2-(thiazol-4-yl)acetate (236 mg, 0.312 mmol, 1 eq.) in DMF (3.5 mL) was added Cs₂CO₃ (204 mg, 0.625 mmol, 2 eq.) at 25° C. After stirring for 30 min, methyl 4′-(bromomethyl)-[1,1′-biphenyl]-2-carboxylate (191 mg, 0.625 mmol, 2 eq.) was added to the reaction mixture. The resulting mixture was stirred at 25° C. for 6 h before it was diluted with H₂O (10 mL) and extracted with EtOAc (3×5 mL). The combined organic layers were washed with brine (10 mL), dried over magnesium sulfate, filtered and concentrated to dryness. The residue was purified by flash column chromatography on SiO₂ (% EtOAc in petroleum ether) to provide methyl 4′-(2-(((2S,4R,5R)-5-(6-(N,N′-bis(tert-butoxycarbonyl)amino)-2-chloro-9H-purin-9-yl)-4-((tert-butyldimethylsilyl)oxy)tetrahydrofuran-2-yl)methoxy)-3-ethoxy-3-oxo-2-(thiazole-4-carbonyl)propyl)-[1,1′-biphenyl]-2-carboxylate (85 mg, 28%) as a white solid.

### Steps 7-9:

To a solution of methyl 4′-(2-(((2S,4R,5R)-5-(6-(N,N′-bis(tert-butoxycarbonyl)-amino)-2-chloro-9H-purin-9-yl)-4-((tert-butyldimethylsilyl)oxy)tetrahydrofuran-2-yl)methoxy)-3-ethoxy-3-oxo-2-(thiazole-4-carbonyl)propyl)-[1,1′-biphenyl]-2-carboxylate (85 mg, 0.087 mmol, 1.0 eq.) in DCM (1.7 mL) and cooled in a wet ice bath, followed by dropwise addition of TFA (100 μL). The reaction was allowed to warm to ambient temperature and stirred for 14 h before it was concentrated to dryness. The resulting oil was dissolved in THF (0.5 mL) at 0° C. and followed by addition of a solution of TBAF (173 μL, 0.173 mmol, 1 M in THF, 2.0 eq.) dropwise. The reaction mixture was stirred from 0° C. to ambient temperature over 4 h before it was evaporated to dryness. The reaction oil was slurried in water (1.0 mL) and cooled in a wet ice bath. 4M NaOH (200 μL, 0.86 mmol, 10.0 eq.) was slowly added. The reaction was allowed to warm to ambient temperature and was held for 10 h. The reaction mixture was adjusted the pH to 2-3 with 1M aq. HCl and then extracted with EtOAc (3×10 mL). The combined organic layer was dried over magnesium sulfate, filtered and concentrated under reduced pressure to give a crude product which was purified to provide the title compound as a mixture of diastereomers (ca. 1:1) and isolated as an off-white solid by preparative reversed-phase HPLC purification.

¹H NMR (CD₃OD, 300 MHz) δ 9.06 (s, 1H), 8.62 (s, 1H), 7.70-7.76 (m, 2H), 7.41-7.50 (m, 3H), 7.11-7.25 (m, 5H), 5.95 (s, 1H), 4.62-4.73 (m, 2H), 3.93 (bs, 2H), 3.54-3.84 (m, 3H), 2.47-2.48 (m, 1H), 2.01 (bs, 3H); LC/MS [M+H]=637.2.

### Example 51

### Assay 1: Inhibition of the CD₇₃ Enzyme In Vitro

For measurements of soluble CD73 enzyme activity, recombinant CD73 was obtained from R&D Systems, Cat. No. 5795-EN-010. Serial dilutions of test compounds were incubated with recombinant CD73 and AMP in reaction buffer (25 mM Tris HCl pH7.5, 5 mM MgCl2, 50 mM NaCl, 0.25 mM DTT, 0.005% Triton X-100). The final reaction volume was 25 μL and the final concentrations of recombinant CD73 and AMP were 0.5 nM and 50 μM, respectively. Reactions were allowed to proceed for 30 minutes at room temperature before the addition of 100 μL Malachite Green (Cell Signaling Technology, Cat. No. 12776). After 5 minutes at room temperature, absorbance at 630 nm was determined on a microplate spectrophotometer. The concentration of inorganic phosphate was determined using a phosphate standard curve.

The IC₅₀ data is given below in Table 2. ND indicates not determined.

TABLE 2

| | | Assay 1 |
|---|---|---|
| | | CD73 |
| Example | | IC50 |
| # | Compound | (nM) |
| 1 | 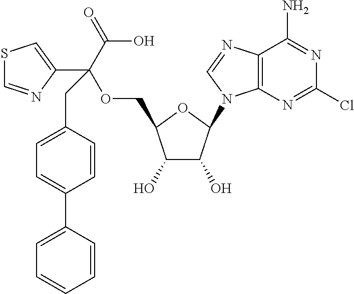 [SMILES: Nc1nc(Cl)nc2c1ncn2[C@@H]1O[C@H](COC(Cc2ccc(-c3ccccc3)cc2)(C(=O)O)c2cscn2)[C@@H](O)[C@H]1O] | 99 |
| 2 | 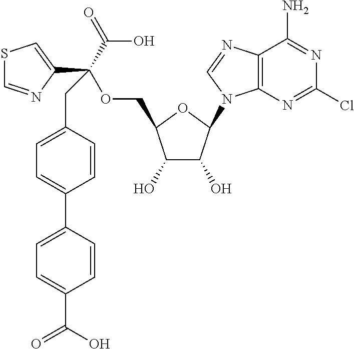 [SMILES: Nc1nc(Cl)nc2c1ncn2[C@@H]1O[C@H](CO[C@](Cc2ccc(-c3ccc(C(=O)O)cc3)cc2)(C(=O)O)c2cscn2)[C@@H](O)[C@H]1O] | 64 |
| 3 | 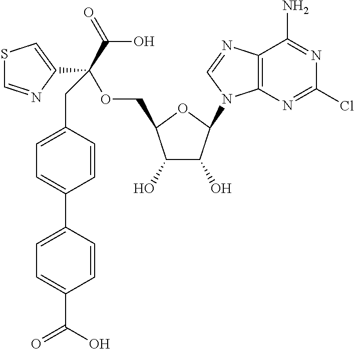 [SMILES: Nc1nc(Cl)nc2c1ncn2[C@@H]1O[C@H](CO[C@@](Cc2ccc(-c3ccc(C(=O)O)cc3)cc2)(C(=O)O)c2cscn2)[C@@H](O)[C@H]1O] | 1251 |
| 4 | 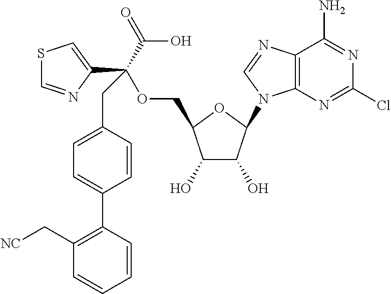 [SMILES: N#CCc1ccccc1-c1ccc(C[C@@](OC[C@H]2O[C@@H](n3cnc4c(N)nc(Cl)nc43)[C@H](O)[C@@H]2O)(C(=O)O)c2cscn2)cc1] | 35 |
| 5 | 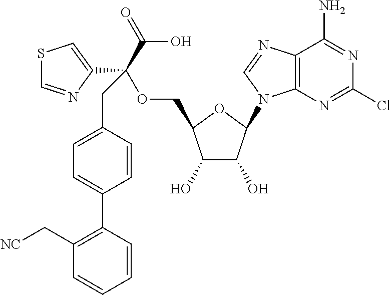 [SMILES: N#CCc1ccccc1-c1ccc(C[C@](OC[C@H]2O[C@@H](n3cnc4c(N)nc(Cl)nc43)[C@H](O)[C@@H]2O)(C(=O)O)c2cscn2)cc1] | 502 |
| 6 | 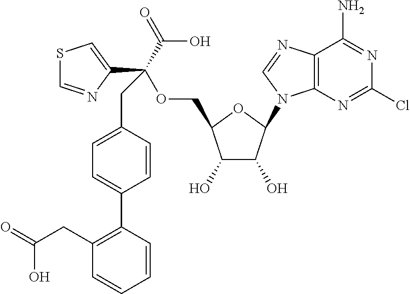 [SMILES: Nc1nc(Cl)nc2c1ncn2[C@@H]1O[C@H](CO[C@](Cc2ccc(-c3ccccc3CC(=O)O)cc2)(C(=O)O)c2cscn2)[C@@H](O)[C@H]1O] | 39 |
| 7 | 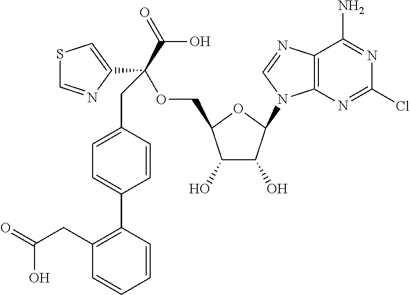 [SMILES: Nc1nc(Cl)nc2c1ncn2[C@@H]1O[C@H](CO[C@@](Cc2ccc(-c3ccccc3CC(=O)O)cc2)(C(=O)O)c2cscn2)[C@@H](O)[C@H]1O] | 1030 |
| 8 | 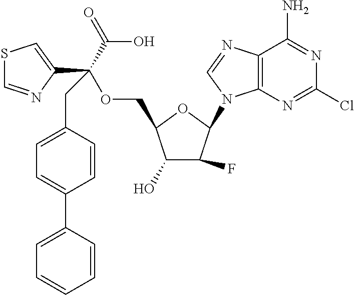 [SMILES: Nc1nc(Cl)nc2c1ncn2[C@@H]1O[C@H](CO[C@](Cc2ccc(-c3ccccc3)cc2)(C(=O)O)c2cscn2)[C@@H](O)[C@@H]1F] | 241 |
| 9 | 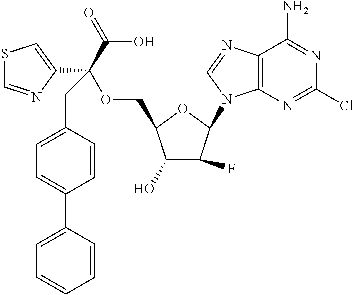 [SMILES: Nc1nc(Cl)nc2c1ncn2[C@@H]1O[C@H](CO[C@@](Cc2ccc(-c3ccccc3)cc2)(C(=O)O)c2cscn2)[C@@H](O)[C@@H]1F] | 2641 |
| 10 | 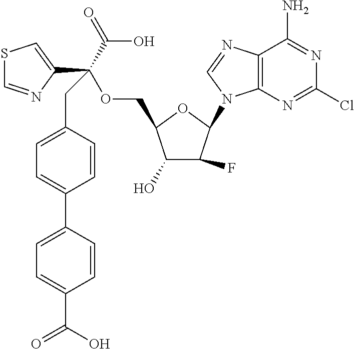 [SMILES: Nc1nc(Cl)nc2c1ncn2[C@@H]1O[C@H](CO[C@](Cc2ccc(-c3ccc(C(=O)O)cc3)cc2)(C(=O)O)c2cscn2)[C@@H](O)[C@@H]1F] | 227 |
| 11 | 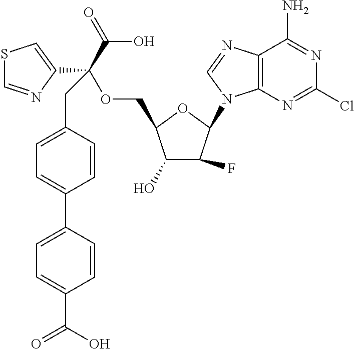 [SMILES: Nc1nc(Cl)nc2c1ncn2[C@@H]1O[C@H](CO[C@@](Cc2ccc(-c3ccc(C(=O)O)cc3)cc2)(C(=O)O)c2cscn2)[C@@H](O)[C@@H]1F] | 1534 |
| 12 | 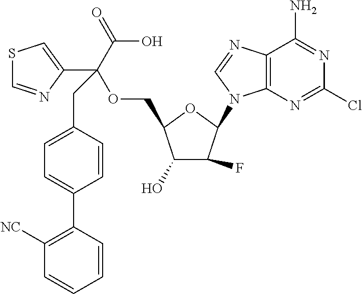 [SMILES: N#Cc1ccccc1-c1ccc(CC(OC[C@H]2O[C@@H](n3cnc4c(N)nc(Cl)nc43)[C@@H](F)[C@@H]2O)(C(=O)O)c2cscn2)cc1] | 433 |
| 13 | 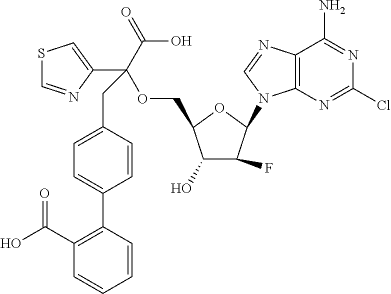 [SMILES: Nc1nc(Cl)nc2c1ncn2[C@@H]1O[C@H](COC(Cc2ccc(-c3ccccc3C(=O)O)cc2)(C(=O)O)c2cscn2)[C@@H](O)[C@@H]1F] | 218 |
| 14 | 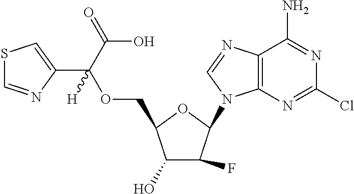 [SMILES: Nc1nc(Cl)nc2c1ncn2[C@@H]1O[C@H](COC(C(=O)O)c2cscn2)[C@@H](O)[C@@H]1F] | 22085 |
| 15 | 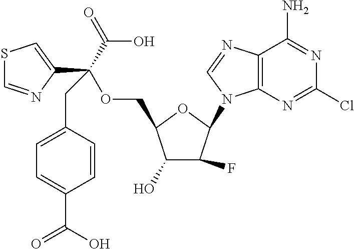 [SMILES: Nc1nc(Cl)nc2c1ncn2[C@@H]1O[C@H](CO[C@](Cc2ccc(C(=O)O)cc2)(C(=O)O)c2cscn2)[C@@H](O)[C@@H]1F] | 296 |
| 16 | 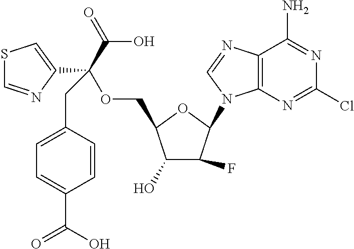 [SMILES: Nc1nc(Cl)nc2c1ncn2[C@@H]1O[C@H](CO[C@@](Cc2ccc(C(=O)O)cc2)(C(=O)O)c2cscn2)[C@@H](O)[C@@H]1F] | 4868 |
| 17 | 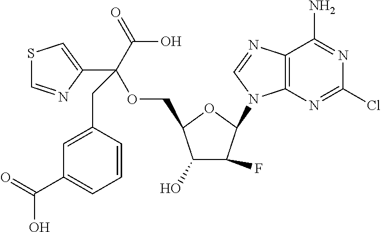 [SMILES: Nc1nc(Cl)nc2c1ncn2[C@@H]1O[C@H](COC(Cc2cccc(C(=O)O)c2)(C(=O)O)c2cscn2)[C@@H](O)[C@@H]1F] | 740 |
| 18 | 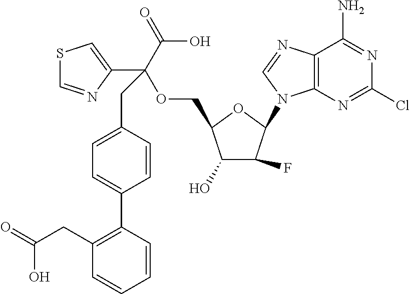 [SMILES: Nc1nc(Cl)nc2c1ncn2[C@@H]1O[C@H](COC(Cc2ccc(-c3ccccc3CC(=O)O)cc2)(C(=O)O)c2cscn2)[C@@H](O)[C@@H]1F] | 182 |
| 19 | 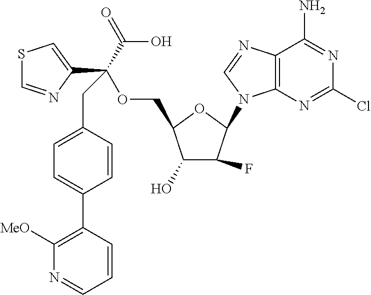 [SMILES: COc1ncccc1-c1ccc(C[C@@](OC[C@H]2O[C@@H](n3cnc4c(N)nc(Cl)nc43)[C@@H](F)[C@@H]2O)(C(=O)O)c2cscn2)cc1] | 170 |
| 20 | 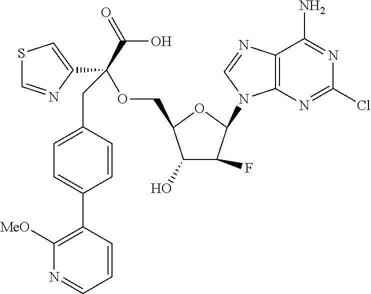 [SMILES: COc1ncccc1-c1ccc(C[C@](OC[C@H]2O[C@@H](n3cnc4c(N)nc(Cl)nc43)[C@@H](F)[C@@H]2O)(C(=O)O)c2cscn2)cc1] | 2754 |
| 21 | 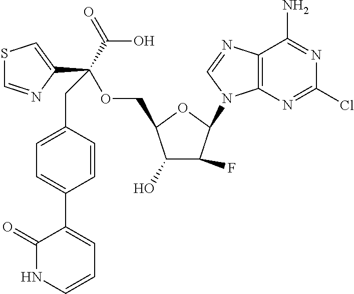 [SMILES: Nc1nc(Cl)nc2c1ncn2[C@@H]1O[C@H](CO[C@](Cc2ccc(-c3ccc[nH]c3=O)cc2)(C(=O)O)c2cscn2)[C@@H](O)[C@@H]1F] | 47 |
| 22 | 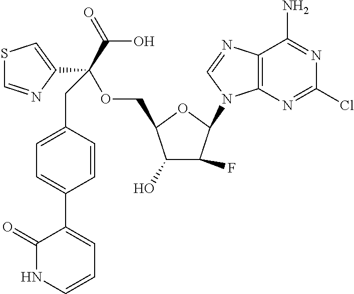 [SMILES: Nc1nc(Cl)nc2c1ncn2[C@@H]1O[C@H](CO[C@@](Cc2ccc(-c3ccc[nH]c3=O)cc2)(C(=O)O)c2cscn2)[C@@H](O)[C@@H]1F] | 2984 |
| 23 | 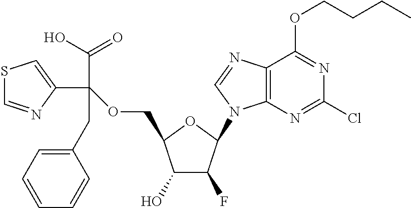 [SMILES: CCCCOc1nc(Cl)nc2c1ncn2[C@@H]1O[C@H](COC(Cc2ccccc2)(C(=O)O)c2cscn2)[C@@H](O)[C@@H]1F] | 3418 |
| 24 | 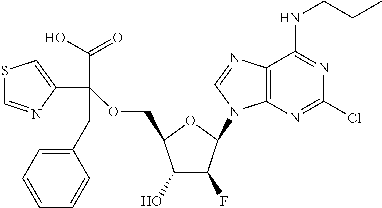 [SMILES: CCCNc1nc(Cl)nc2c1ncn2[C@@H]1O[C@H](COC(Cc2ccccc2)(C(=O)O)c2cscn2)[C@@H](O)[C@@H]1F] | 1241 |
| 25 | 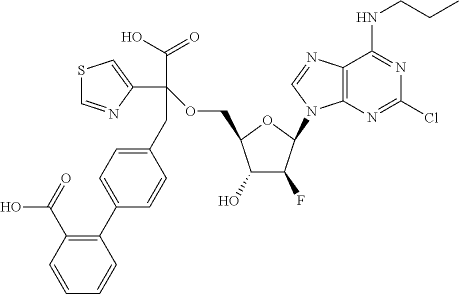 [SMILES: CCCNc1nc(Cl)nc2c1ncn2[C@@H]1O[C@H](COC(Cc2ccc(-c3ccccc3C(=O)O)cc2)(C(=O)O)c2cscn2)[C@@H](O)[C@@H]1F] | 382 |
| 26 | 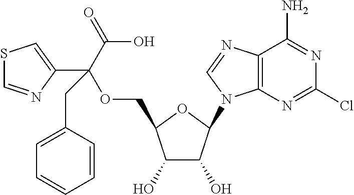 [SMILES: Nc1nc(Cl)nc2c1ncn2[C@@H]1O[C@H](COC(Cc2ccccc2)(C(=O)O)c2cscn2)[C@@H](O)[C@H]1O] | 400 |
| 27 | 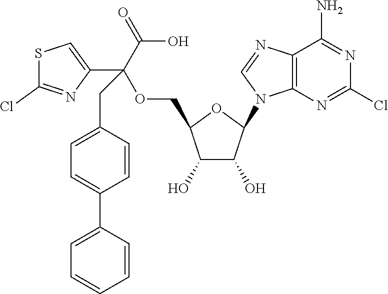 [SMILES: Nc1nc(Cl)nc2c1ncn2[C@@H]1O[C@H](COC(Cc2ccc(-c3ccccc3)cc2)(C(=O)O)c2csc(Cl)n2)[C@@H](O)[C@H]1O] | 106 |
| 28 | 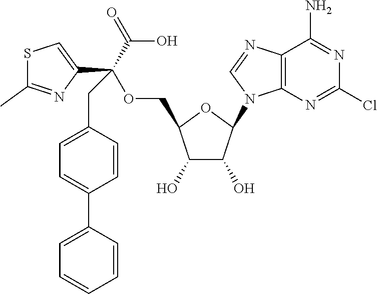 [SMILES: Cc1nc([C@](Cc2ccc(-c3ccccc3)cc2)(OC[C@H]2O[C@@H](n3cnc4c(N)nc(Cl)nc43)[C@H](O)[C@@H]2O)C(=O)O)cs1] | 90 |
| 29 | 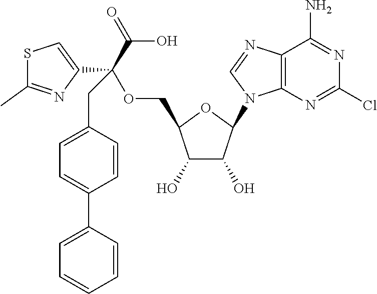 [SMILES: Cc1nc([C@@](Cc2ccc(-c3ccccc3)cc2)(OC[C@H]2O[C@@H](n3cnc4c(N)nc(Cl)nc43)[C@H](O)[C@@H]2O)C(=O)O)cs1] | 3222 |
| 30 | 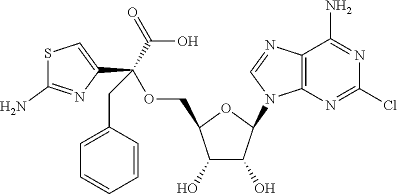 [SMILES: Nc1nc([C@](Cc2ccccc2)(OC[C@H]2O[C@@H](n3cnc4c(N)nc(Cl)nc43)[C@H](O)[C@@H]2O)C(=O)O)cs1] | 342 |
| 31 | 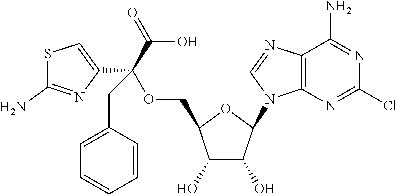 [SMILES: Nc1nc([C@@](Cc2ccccc2)(OC[C@H]2O[C@@H](n3cnc4c(N)nc(Cl)nc43)[C@H](O)[C@@H]2O)C(=O)O)cs1] | 1972 |
| 32 | 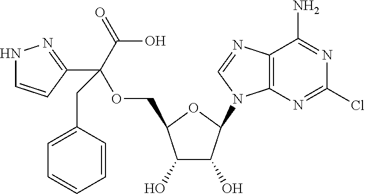 [SMILES: Nc1nc(Cl)nc2c1ncn2[C@@H]1O[C@H](COC(Cc2ccccc2)(C(=O)O)c2cc[nH]n2)[C@@H](O)[C@H]1O] | 2057 |
| 33 | 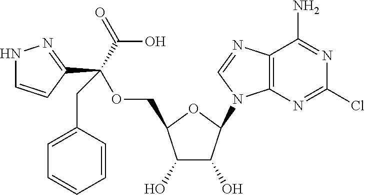 [SMILES: Nc1nc(Cl)nc2c1ncn2[C@@H]1O[C@H](CO[C@](Cc2ccccc2)(C(=O)O)c2cc[nH]n2)[C@@H](O)[C@H]1O] | 420 |
| 34 | 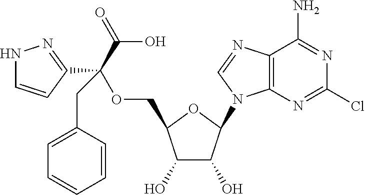 [SMILES: Nc1nc(Cl)nc2c1ncn2[C@@H]1O[C@H](CO[C@@](Cc2ccccc2)(C(=O)O)c2cc[nH]n2)[C@@H](O)[C@H]1O] | 5864 |
| 35 | 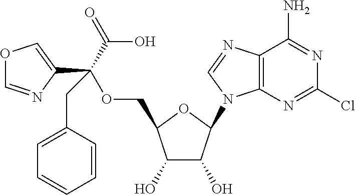 [SMILES: Nc1nc(Cl)nc2c1ncn2[C@@H]1O[C@H](CO[C@](Cc2ccccc2)(C(=O)O)c2cocn2)[C@@H](O)[C@H]1O] | 290 |
| 36 | 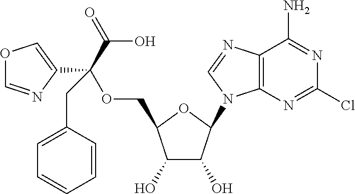 [SMILES: Nc1nc(Cl)nc2c1ncn2[C@@H]1O[C@H](CO[C@@](Cc2ccccc2)(C(=O)O)c2cocn2)[C@@H](O)[C@H]1O] | 6262 |
| 37 | 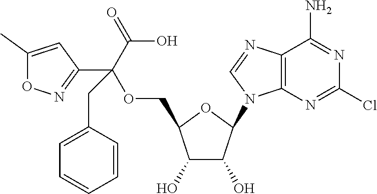 [SMILES: Cc1cc(C(Cc2ccccc2)(OC[C@H]2O[C@@H](n3cnc4c(N)nc(Cl)nc43)[C@H](O)[C@@H]2O)C(=O)O)no1] | 8734 |
| 38 | 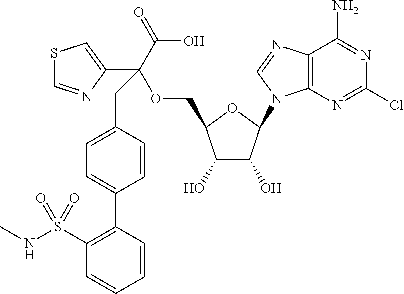 [SMILES: CNS(=O)(=O)c1ccccc1-c1ccc(CC(OC[C@H]2O[C@@H](n3cnc4c(N)nc(Cl)nc43)[C@H](O)[C@@H]2O)(C(=O)O)c2cscn2)cc1] | 42 |
| 39 | 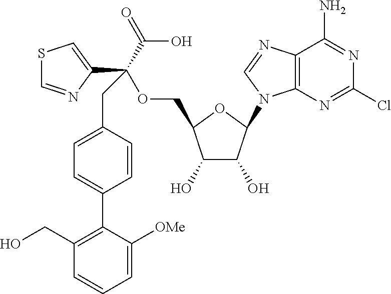 [SMILES: COc1cccc(CO)c1-c1ccc(C[C@@](OC[C@H]2O[C@@H](n3cnc4c(N)nc(Cl)nc43)[C@H](O)[C@@H]2O)(C(=O)O)c2cscn2)cc1] | 68 |
| 40 | 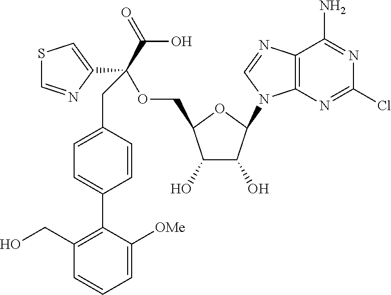 [SMILES: COc1cccc(CO)c1-c1ccc(C[C@](OC[C@H]2O[C@@H](n3cnc4c(N)nc(Cl)nc43)[C@H](O)[C@@H]2O)(C(=O)O)c2cscn2)cc1] | 2227 |
| 41 | 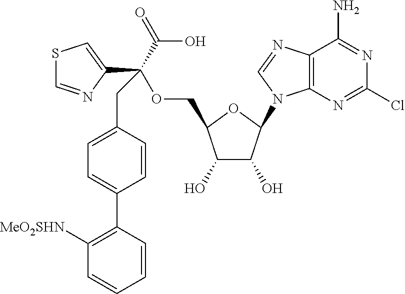 | 43 |
| 42 | 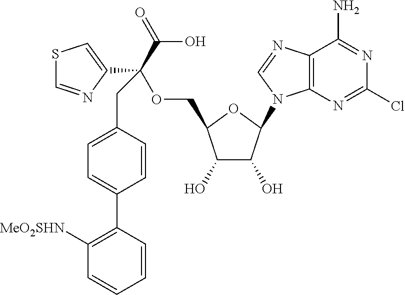 | 904 |
| 43 | 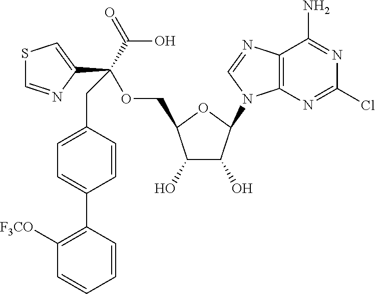 [SMILES: Nc1nc(Cl)nc2c1ncn2[C@@H]1O[C@H](CO[C@](Cc2ccc(-c3ccccc3OC(F)(F)F)cc2)(C(=O)O)c2cscn2)[C@@H](O)[C@H]1O] | 51 |
| 44 | 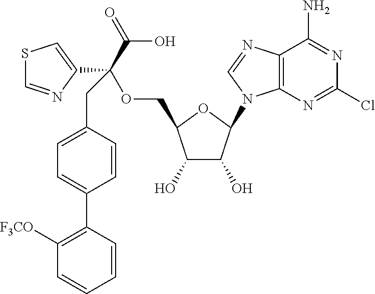 [SMILES: Nc1nc(Cl)nc2c1ncn2[C@@H]1O[C@H](CO[C@@](Cc2ccc(-c3ccccc3OC(F)(F)F)cc2)(C(=O)O)c2cscn2)[C@@H](O)[C@H]1O] | 365 |
| 45 | 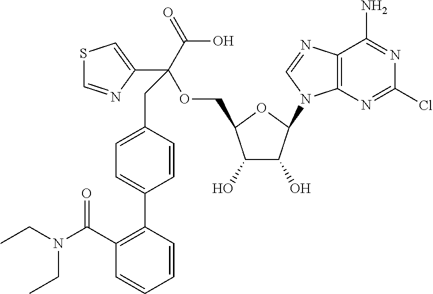 [SMILES: CCN(CC)C(=O)c1ccccc1-c1ccc(CC(OC[C@H]2O[C@@H](n3cnc4c(N)nc(Cl)nc43)[C@H](O)[C@@H]2O)(C(=O)O)c2cscn2)cc1] | 833 |
| 46 | 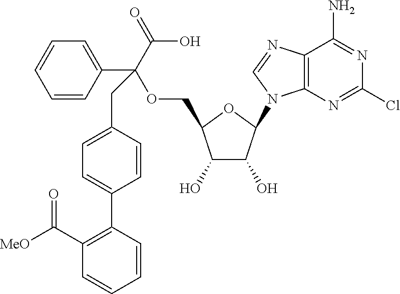 [SMILES: COC(=O)c1ccccc1-c1ccc(CC(OC[C@H]2O[C@@H](n3cnc4c(N)nc(Cl)nc43)[C@H](O)[C@@H]2O)(C(=O)O)c2ccccc2)cc1] | >50000 |
| 47 | 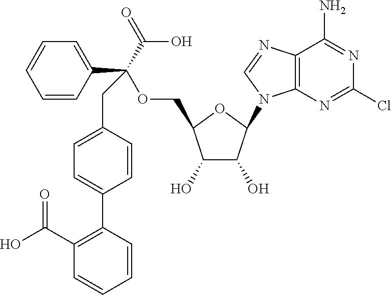 [SMILES: Nc1nc(Cl)nc2c1ncn2[C@@H]1O[C@H](CO[C@](Cc2ccc(-c3ccccc3C(=O)O)cc2)(C(=O)O)c2ccccc2)[C@@H](O)[C@H]1O] | 4220 |
| 48 | 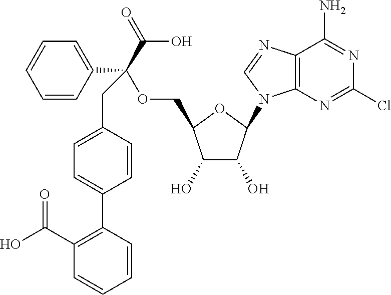 [SMILES: Nc1nc(Cl)nc2c1ncn2[C@@H]1O[C@H](CO[C@@](Cc2ccc(-c3ccccc3C(=O)O)cc2)(C(=O)O)c2ccccc2)[C@@H](O)[C@H]1O] | 14105 |
| 49 | 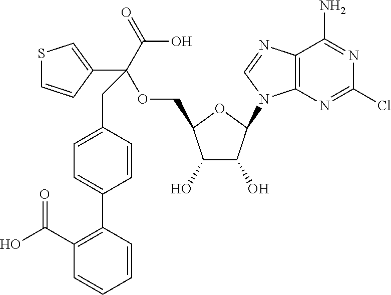 [SMILES: Nc1nc(Cl)nc2c1ncn2[C@@H]1O[C@H](COC(Cc2ccc(-c3ccccc3C(=O)O)cc2)(C(=O)O)c2ccsc2)[C@@H](O)[C@H]1O] | 2268 |
| 50 | 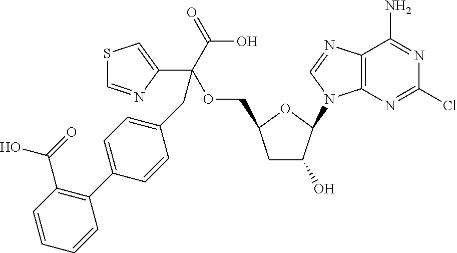 [SMILES: Nc1nc(Cl)nc2c1ncn2[C@@H]1O[C@H](COC(Cc2ccc(-c3ccccc3C(=O)O)cc2)(C(=O)O)c2cscn2)C[C@H]1O] | >1000 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below.

The full scope of the invention should be determined by reference to the claims, along with their full scope of equivalents, and the specification, along with such variations.

1. A compound of formula (I): 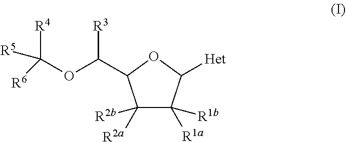
or a pharmaceutically acceptable salt and/or prodrug thereof, wherein
Het is heterocyclyl or heteroaryl;
R^{1a} is selected from H, halo, hydroxy, cyano, azido, amino, —O—C(O)—O—C₁₋₆alkyl, C₁₋₆acyloxy, and C₁₋₆alkoxy;
R^{1b} is selected from H and halo;
R^{2a} is selected from H, halo, hydroxy, cyano, azido, amino, C₁₋₆acyloxy, —O—C(O)—O—C₁₋₆alkyl, and C₁₋₆alkoxy;
R^{2b} is selected from H and halo;
R³ is selected from H and alkyl;
R⁴ is selected from aryl and heteroaryl;
R⁵ is selected from aralkyl and heteroaralkyl;
R⁶ is selected from —C(O)OR⁹, —C(O)NR¹³R¹⁴, —S(O)₂R¹⁰ and —P(O)(OR¹¹)(OR¹²);
R⁹ is independently selected from H, alkyl, cycloalkyl, cycloalkylalkyl, heterocyclyl, heterocyclylalkyl, aryl, aralkyl, heteroaryl, and heteroaralkyl;
R¹⁰ is independently selected from alkyl, alkenyl, alkynyl, amino, cycloalkyl, cycloalkylalkyl, heterocyclyl, heterocyclylalkyl, aryl, aralkyl, heteroaryl, and heteroaralkyl; and
R¹¹, R¹² and R¹⁴ are independently selected from H, alkyl, cycloalkyl, cycloalkylalkyl, heterocyclyl, heterocyclylalkyl, aryl, aralkyl, heteroaryl, heteroaralkyl; and
R¹³ is selected from H, hydroxy, alkyl, cycloalkyl, cycloalkylalkyl, heterocyclyl, heterocyclylalkyl, aryl, aralkyl, heteroaryl, heteroaralkyl;
provided that
if R⁴ is unsubstituted or substituted tetrazolyl, and
R⁶ is —C(O)OR⁹, then
R⁵ is not unsubstituted —CH₂-pyridyl, unsubstituted —CH₂-thienyl, —CH₂-thienyl substituted with a —C(O)OH group, unsubstituted benzyl, or benzyl substituted with a trifluoromethyl, trifluoromethoxy, methoxycarbonyl, —C(O)OH, benzyloxy, or phenyl group.

2. The compound of claim 1, wherein R^{1a} is H or hydroxy.

3. The compound of claim 1 or 2, wherein R^{1b} is H.

4. The compound of claim 1, wherein R^{1a} is H and R^{1b} is halo, preferably F.

5. The compound of any one of claims 1-4, wherein R^{2a} is H or hydroxy, preferably hydroxy.

6. The compound of any one of claims 1-5, wherein R^{2b} is H.

7. The compound of claim 1, wherein R^{1a} is hydroxy, R^{1b} is H, R^{2a} is hydroxy, and R^{2b} is H.

8. The compound of any preceding claim, having the structure:

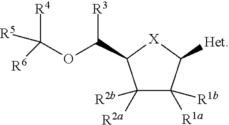

9. The compound of any preceding claim, wherein R^{1a} is in the α-configuration.

10. The compound of claim 9, wherein the compound of Formula (I) has the structure (IA):

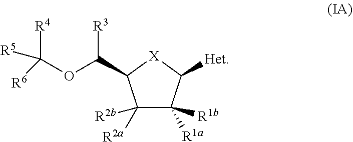

11. The compound of any one of claims 1-8, wherein R^{1a} is in the β-configuration.

12. The compound of claim 11, wherein the compound of Formula (I) has the structure (IB):

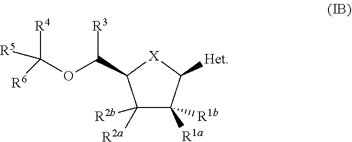

13. The compound of any preceding claim, wherein R^{2a} is in the α-configuration.

14. The compound of claim 13, wherein the compound of Formula (I) has the structure (IC):

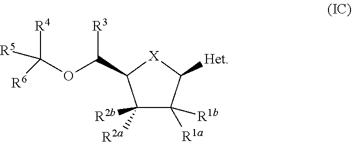

15. The compound of any one of claims 1-12, wherein R^{2a} is in the β-configuration.

16. The compound of claim 15, wherein the compound of Formula (I) has the structure (ID):

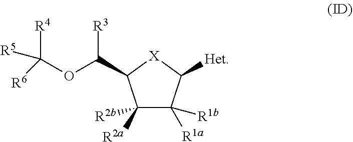

17. The compound of claim 8, wherein the compound of Formula (I) has the structure (IE):

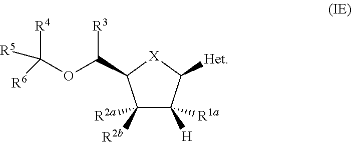

18. The compound of any preceding claim, wherein R³ is H.

19. The compound of claim 18, wherein R⁴ is thiazolyl, pyrazolyl, triazolyl, oxazolyl, or thienyl.

20. The compound of any preceding claim, wherein R⁵ is aralkyl, preferably benzyl.

21. The compound of claim 20, wherein R⁵ is aralkyl or heteroaralkyl unsubstituted or substituted with one or more substituents selected from carboxy, heteroaryl, and aryl, preferably aryl or heteroaryl.

22. The compound of claim 21, wherein R⁵ is aralkyl substituted on the aryl ring (e.g., a benzyl substituted at a para-position of the phenyl ring) with a second aryl or heteroaryl ring (preferably a phenyl ring) unsubstituted or substituted with one or more substituents, e.g., selected from hydroxyl, cyano, alkyl, alkoxy, amido, carboxy, alkoxycarbonyl, heterocyclyl, heteroaryl, and sulfonamido.

23. The compound of claim 22, wherein R⁵ is benzyl substituted on the phenyl ring (e.g., at the 4-position) with

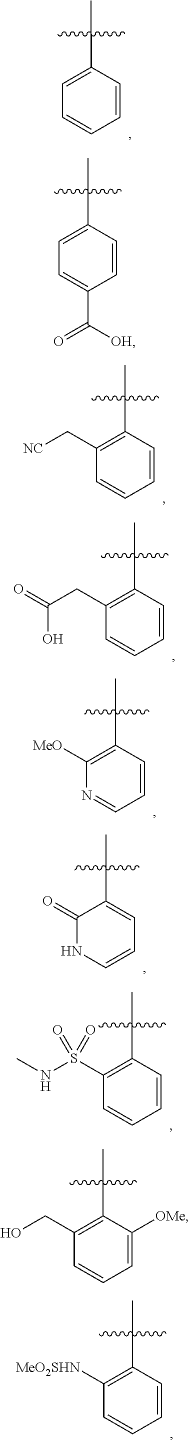

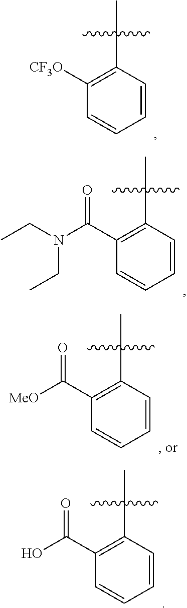

24. The compound of any preceding claim, wherein R⁶ is —C(O)OR⁹ and R⁹ is H or alkyl.

25. The compound of any one of claims 1-16, wherein

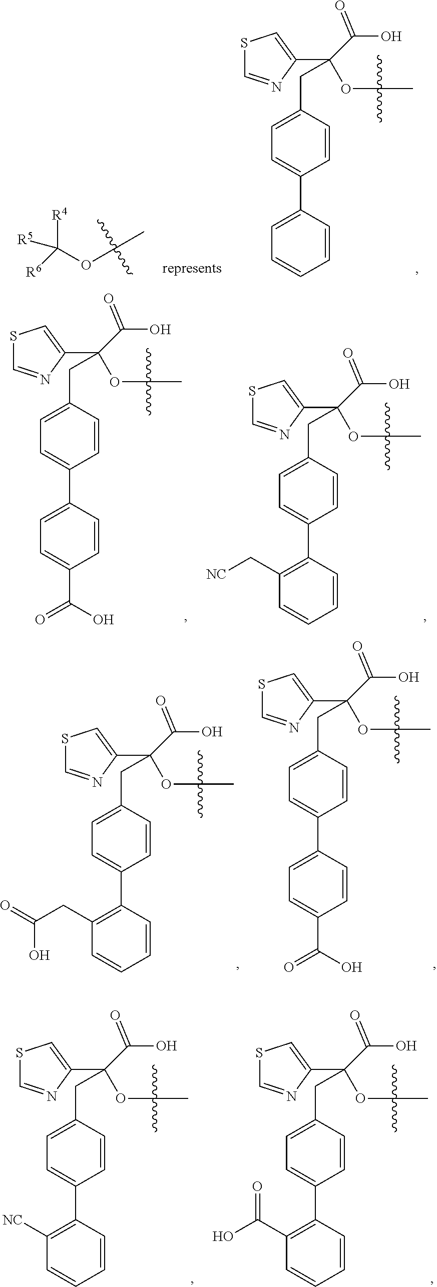

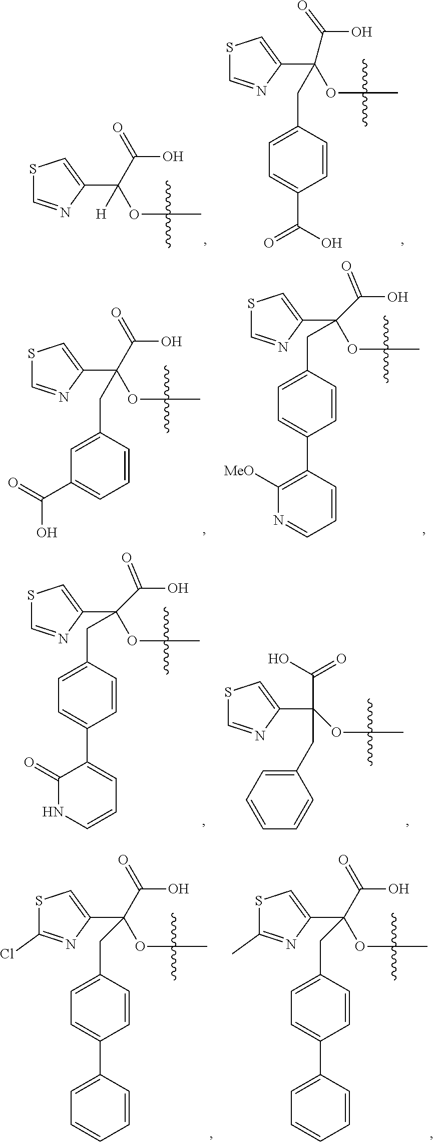

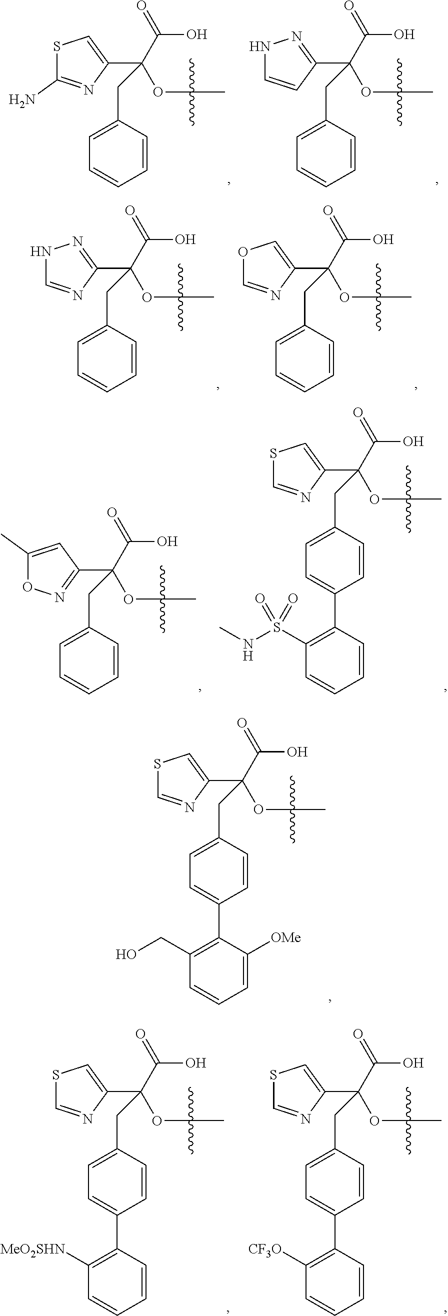

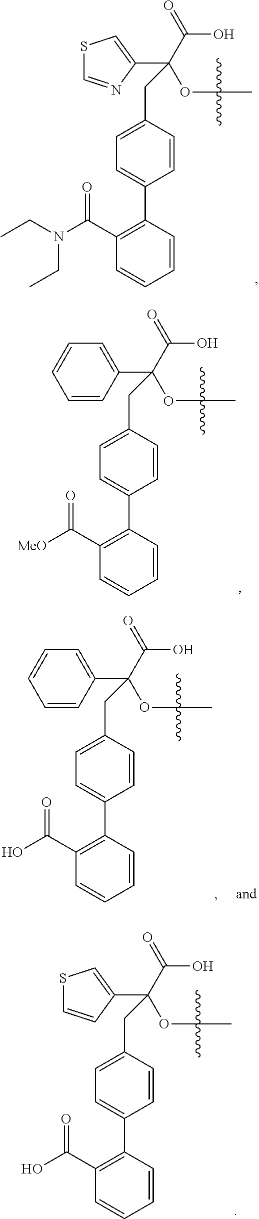

26. The compound of any preceding claim, wherein R⁹ is H or C₁₋₆alkyl.

27. The compound of any preceding claim, wherein Het is selected from a 6- to 10-membered aryl, a 5- to 8-membered heterocyclyl, a 5- to 8-membered monocyclic or 5- to 10-membered bicyclic heteroaryl and is unsubstituted or substituted with one or more substituents selected from halo, alkoxy, and amino.

28. The compound of claim 27, wherein the Het substituents are selected from halo and amino.

29. The compound of claim 27, wherein Het is a nitrogen-containing heterocyclyl or heteroaryl.

30. The compound of claim 27, wherein, Het is 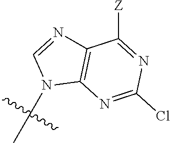
wherein
Z is OR⁷ or NR⁷R⁸
R⁷ is selected from H, alkyl, aralkyl, heteroaralkyl, cycloalkyl, and heterocyclyl; and
R⁸ is H or alkyl.

31. The compound of claim 30, wherein R⁷ is alkyl and R⁸ is H.

32. A compound selected from:

| Example # | Compound |
|---|---|
| 1 | 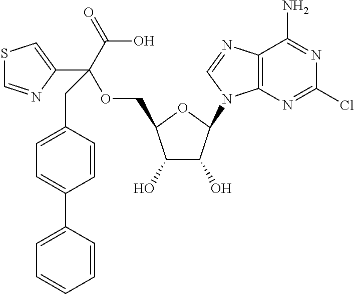 [SMILES: Nc1nc(Cl)nc2c1ncn2[C@@H]1O[C@H](COC(Cc2ccc(-c3ccccc3)cc2)(C(=O)O)c2cscn2)[C@@H](O)[C@H]1O] |
| 2 | 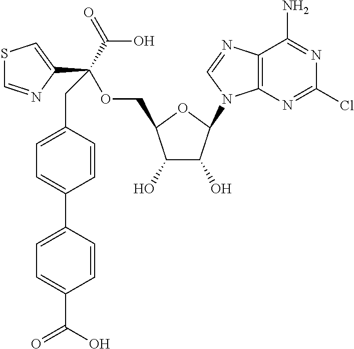 [SMILES: Nc1nc(Cl)nc2c1ncn2[C@@H]1O[C@H](CO[C@](Cc2ccc(-c3ccc(C(=O)O)cc3)cc2)(C(=O)O)c2cscn2)[C@@H](O)[C@H]1O] |
| 3 | 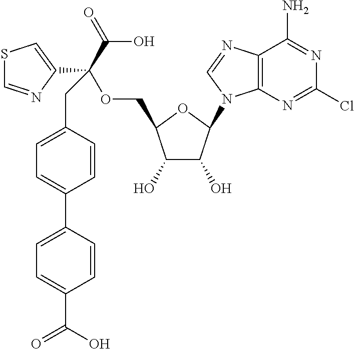 [SMILES: Nc1nc(Cl)nc2c1ncn2[C@@H]1O[C@H](CO[C@@](Cc2ccc(-c3ccc(C(=O)O)cc3)cc2)(C(=O)O)c2cscn2)[C@@H](O)[C@H]1O] |
| 4 | 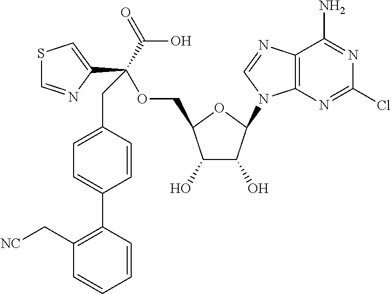 [SMILES: N#CCc1ccccc1-c1ccc(C[C@@](OC[C@H]2O[C@@H](n3cnc4c(N)nc(Cl)nc43)[C@H](O)[C@@H]2O)(C(=O)O)c2cscn2)cc1] |
| 5 | 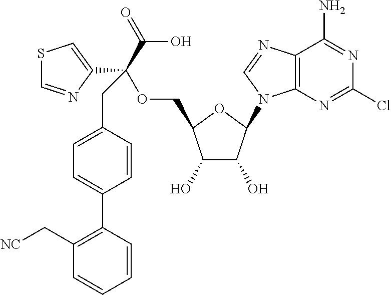 [SMILES: N#CCc1ccccc1-c1ccc(C[C@](OC[C@H]2O[C@@H](n3cnc4c(N)nc(Cl)nc43)[C@H](O)[C@@H]2O)(C(=O)O)c2cscn2)cc1] |
| 6 | 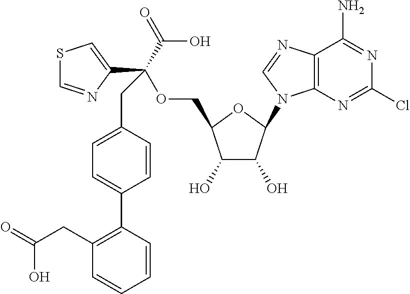 [SMILES: Nc1nc(Cl)nc2c1ncn2[C@@H]1O[C@H](CO[C@](Cc2ccc(-c3ccccc3CC(=O)O)cc2)(C(=O)O)c2cscn2)[C@@H](O)[C@H]1O] |
| 7 | 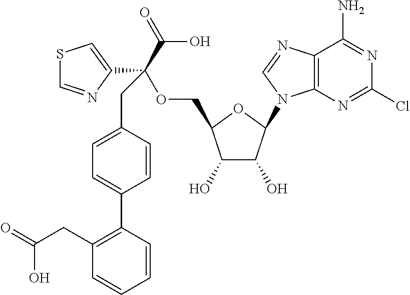 [SMILES: Nc1nc(Cl)nc2c1ncn2[C@@H]1O[C@H](CO[C@@](Cc2ccc(-c3ccccc3CC(=O)O)cc2)(C(=O)O)c2cscn2)[C@@H](O)[C@H]1O] |
| 8 | 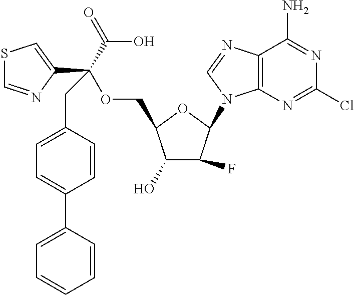 [SMILES: Nc1nc(Cl)nc2c1ncn2[C@@H]1O[C@H](CO[C@](Cc2ccc(-c3ccccc3)cc2)(C(=O)O)c2cscn2)[C@@H](O)[C@@H]1F] |
| 9 | 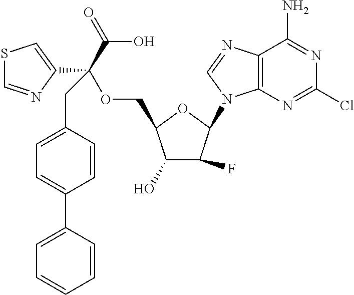 [SMILES: Nc1nc(Cl)nc2c1ncn2[C@@H]1O[C@H](CO[C@@](Cc2ccc(-c3ccccc3)cc2)(C(=O)O)c2cscn2)[C@@H](O)[C@@H]1F] |
| 10 | 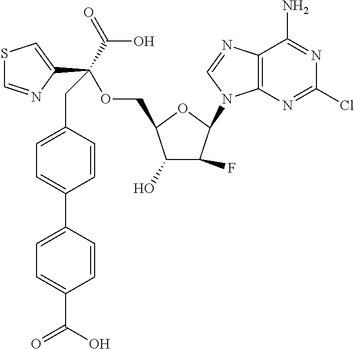 [SMILES: Nc1nc(Cl)nc2c1ncn2[C@@H]1O[C@H](CO[C@](Cc2ccc(-c3ccc(C(=O)O)cc3)cc2)(C(=O)O)c2cscn2)[C@@H](O)[C@@H]1F] |
| 11 | 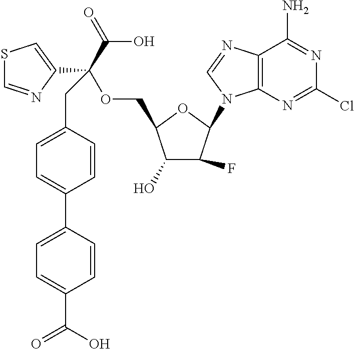 [SMILES: Nc1nc(Cl)nc2c1ncn2[C@@H]1O[C@H](CO[C@@](Cc2ccc(-c3ccc(C(=O)O)cc3)cc2)(C(=O)O)c2cscn2)[C@@H](O)[C@@H]1F] |
| 12 | 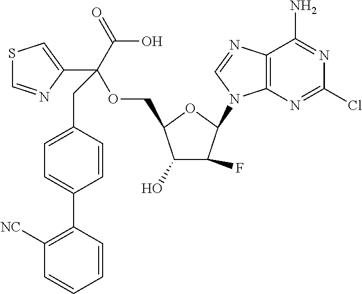 [SMILES: N#Cc1ccccc1-c1ccc(CC(OC[C@H]2O[C@@H](n3cnc4c(N)nc(Cl)nc43)[C@@H](F)[C@@H]2O)(C(=O)O)c2cscn2)cc1] |
| 13 | 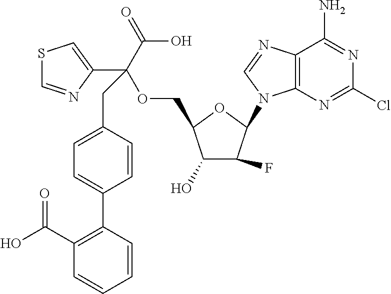 [SMILES: Nc1nc(Cl)nc2c1ncn2[C@@H]1O[C@H](COC(Cc2ccc(-c3ccccc3C(=O)O)cc2)(C(=O)O)c2cscn2)[C@@H](O)[C@@H]1F] |
| 14 | 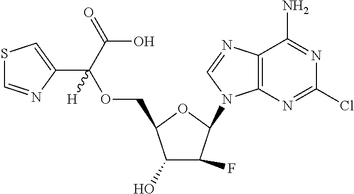 [SMILES: Nc1nc(Cl)nc2c1ncn2[C@@H]1O[C@H](COC(C(=O)O)c2cscn2)[C@@H](O)[C@@H]1F] |
| 15 | 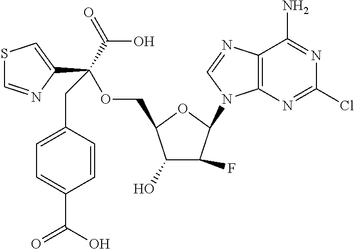 [SMILES: Nc1nc(Cl)nc2c1ncn2[C@@H]1O[C@H](CO[C@](Cc2ccc(C(=O)O)cc2)(C(=O)O)c2cscn2)[C@@H](O)[C@@H]1F] |
| 16 | 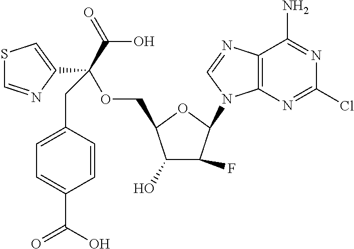 [SMILES: Nc1nc(Cl)nc2c1ncn2[C@@H]1O[C@H](CO[C@@](Cc2ccc(C(=O)O)cc2)(C(=O)O)c2cscn2)[C@@H](O)[C@@H]1F] |
| 17 | 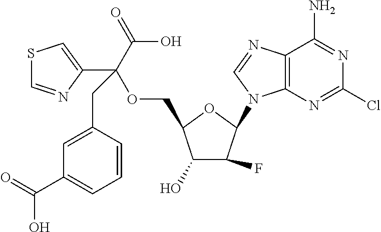 [SMILES: Nc1nc(Cl)nc2c1ncn2[C@@H]1O[C@H](COC(Cc2cccc(C(=O)O)c2)(C(=O)O)c2cscn2)[C@@H](O)[C@@H]1F] |
| 18 | 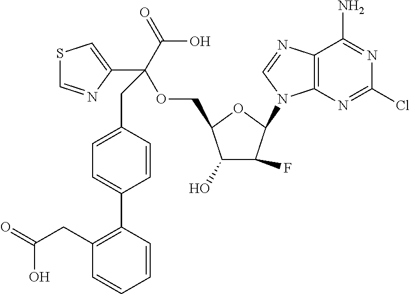 [SMILES: Nc1nc(Cl)nc2c1ncn2[C@@H]1O[C@H](COC(Cc2ccc(-c3ccccc3CC(=O)O)cc2)(C(=O)O)c2cscn2)[C@@H](O)[C@@H]1F] |
| 19 | 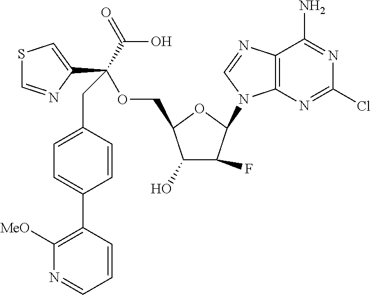 [SMILES: COc1ncccc1-c1ccc(C[C@@](OC[C@H]2O[C@@H](n3cnc4c(N)nc(Cl)nc43)[C@@H](F)[C@@H]2O)(C(=O)O)c2cscn2)cc1] |
| 20 | 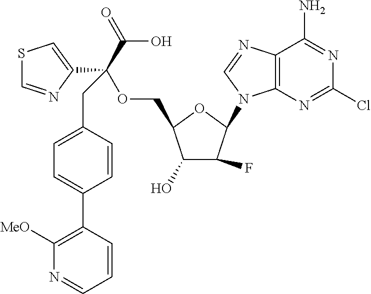 [SMILES: COc1ncccc1-c1ccc(C[C@](OC[C@H]2O[C@@H](n3cnc4c(N)nc(Cl)nc43)[C@@H](F)[C@@H]2O)(C(=O)O)c2cscn2)cc1] |
| 21 | 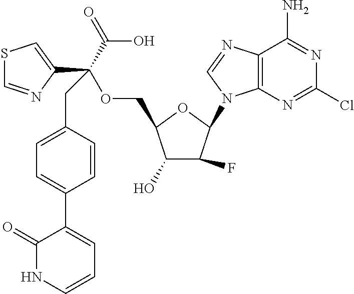 [SMILES: Nc1nc(Cl)nc2c1ncn2[C@@H]1O[C@H](CO[C@](Cc2ccc(-c3ccc[nH]c3=O)cc2)(C(=O)O)c2cscn2)[C@@H](O)[C@@H]1F] |
| 22 | 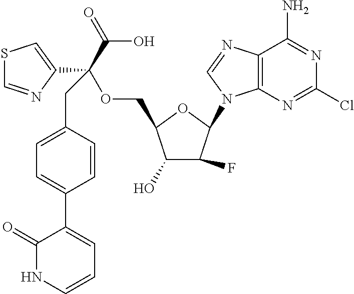 [SMILES: Nc1nc(Cl)nc2c1ncn2[C@@H]1O[C@H](CO[C@@](Cc2ccc(-c3ccc[nH]c3=O)cc2)(C(=O)O)c2cscn2)[C@@H](O)[C@@H]1F] |
| 23 | 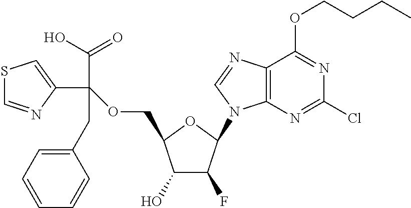 [SMILES: CCCCOc1nc(Cl)nc2c1ncn2[C@@H]1O[C@H](COC(Cc2ccccc2)(C(=O)O)c2cscn2)[C@@H](O)[C@@H]1F] |
| 24 | 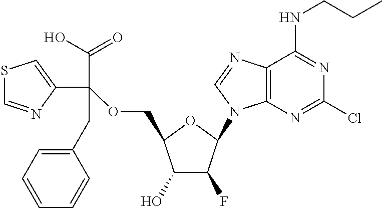 [SMILES: CCCNc1nc(Cl)nc2c1ncn2[C@@H]1O[C@H](COC(Cc2ccccc2)(C(=O)O)c2cscn2)[C@@H](O)[C@@H]1F] |
| 25 | 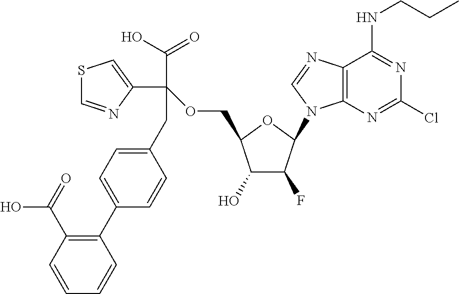 [SMILES: CCCNc1nc(Cl)nc2c1ncn2[C@@H]1O[C@H](COC(Cc2ccc(-c3ccccc3C(=O)O)cc2)(C(=O)O)c2cscn2)[C@@H](O)[C@@H]1F] |
| 26 | 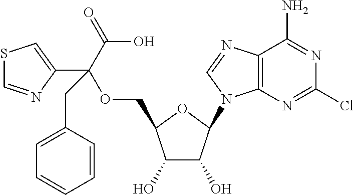 [SMILES: Nc1nc(Cl)nc2c1ncn2[C@@H]1O[C@H](COC(Cc2ccccc2)(C(=O)O)c2cscn2)[C@@H](O)[C@H]1O] |
| 27 | 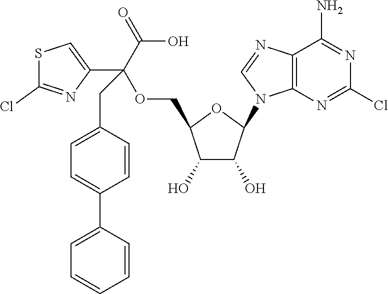 [SMILES: Nc1nc(Cl)nc2c1ncn2[C@@H]1O[C@H](COC(Cc2ccc(-c3ccccc3)cc2)(C(=O)O)c2csc(Cl)n2)[C@@H](O)[C@H]1O] |
| 28 | 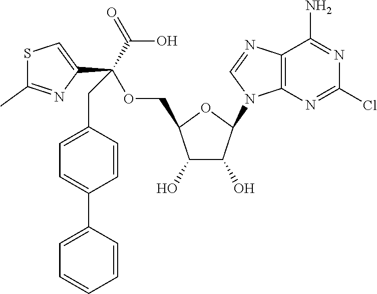 [SMILES: Cc1nc([C@](Cc2ccc(-c3ccccc3)cc2)(OC[C@H]2O[C@@H](n3cnc4c(N)nc(Cl)nc43)[C@H](O)[C@@H]2O)C(=O)O)cs1] |
| 29 | 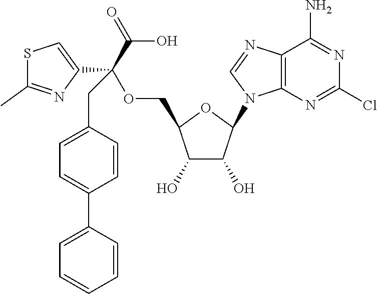 [SMILES: Cc1nc([C@@](Cc2ccc(-c3ccccc3)cc2)(OC[C@H]2O[C@@H](n3cnc4c(N)nc(Cl)nc43)[C@H](O)[C@@H]2O)C(=O)O)cs1] |
| 30 | 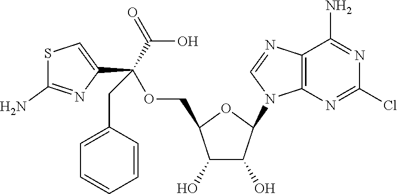 [SMILES: Nc1nc([C@](Cc2ccccc2)(OC[C@H]2O[C@@H](n3cnc4c(N)nc(Cl)nc43)[C@H](O)[C@@H]2O)C(=O)O)cs1] |
| 31 | 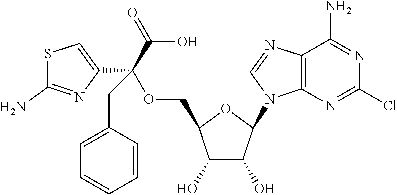 [SMILES: Nc1nc([C@@](Cc2ccccc2)(OC[C@H]2O[C@@H](n3cnc4c(N)nc(Cl)nc43)[C@H](O)[C@@H]2O)C(=O)O)cs1] |
| 32 | 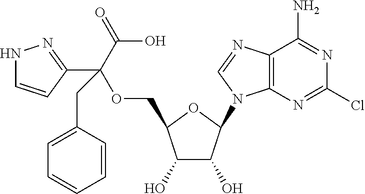 [SMILES: Nc1nc(Cl)nc2c1ncn2[C@@H]1O[C@H](COC(Cc2ccccc2)(C(=O)O)c2cc[nH]n2)[C@@H](O)[C@H]1O] |
| 33 | 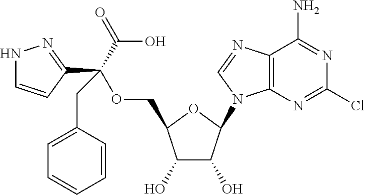 [SMILES: Nc1nc(Cl)nc2c1ncn2[C@@H]1O[C@H](CO[C@](Cc2ccccc2)(C(=O)O)c2cc[nH]n2)[C@@H](O)[C@H]1O] |
| 34 | 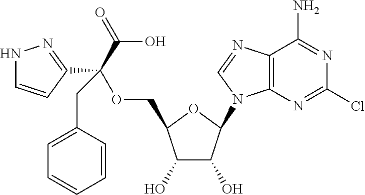 [SMILES: Nc1nc(Cl)nc2c1ncn2[C@@H]1O[C@H](CO[C@@](Cc2ccccc2)(C(=O)O)c2cc[nH]n2)[C@@H](O)[C@H]1O] |
| 35 | 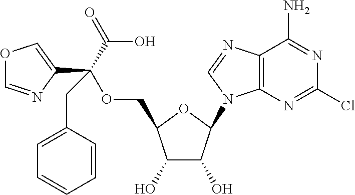 [SMILES: Nc1nc(Cl)nc2c1ncn2[C@@H]1O[C@H](CO[C@](Cc2ccccc2)(C(=O)O)c2cocn2)[C@@H](O)[C@H]1O] |
| 36 | 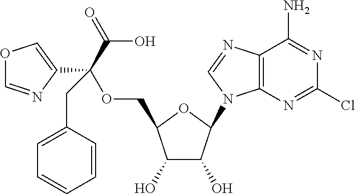 [SMILES: Nc1nc(Cl)nc2c1ncn2[C@@H]1O[C@H](CO[C@@](Cc2ccccc2)(C(=O)O)c2cocn2)[C@@H](O)[C@H]1O] |
| 37 | 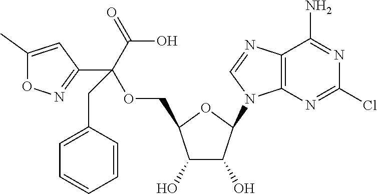 [SMILES: Cc1cc(C(Cc2ccccc2)(OC[C@H]2O[C@@H](n3cnc4c(N)nc(Cl)nc43)[C@H](O)[C@@H]2O)C(=O)O)no1] |
| 38 | 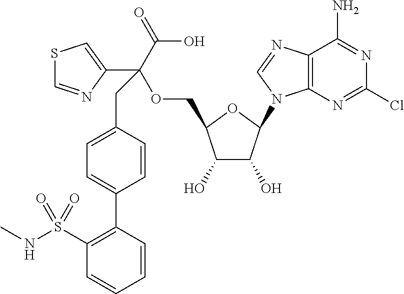 [SMILES: CNS(=O)(=O)c1ccccc1-c1ccc(CC(OC[C@H]2O[C@@H](n3cnc4c(N)nc(Cl)nc43)[C@H](O)[C@@H]2O)(C(=O)O)c2cscn2)cc1] |
| 39 | 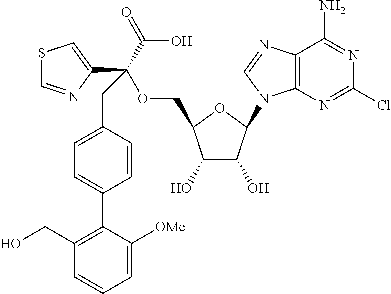 [SMILES: COc1cccc(CO)c1-c1ccc(C[C@@](OC[C@H]2O[C@@H](n3cnc4c(N)nc(Cl)nc43)[C@H](O)[C@@H]2O)(C(=O)O)c2cscn2)cc1] |
| 40 | 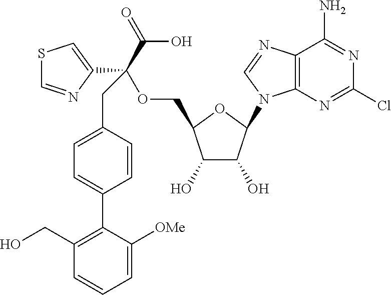 [SMILES: COc1cccc(CO)c1-c1ccc(C[C@](OC[C@H]2O[C@@H](n3cnc4c(N)nc(Cl)nc43)[C@H](O)[C@@H]2O)(C(=O)O)c2cscn2)cc1] |
| 41 | 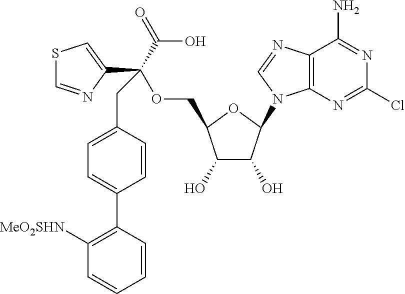 |
| 42 | 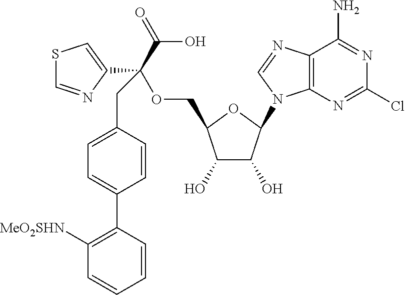 |
| 43 | 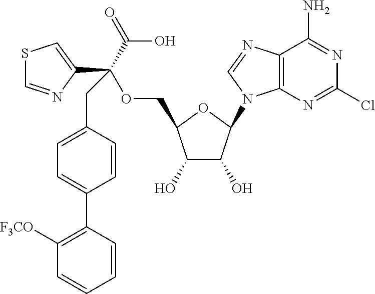 [SMILES: Nc1nc(Cl)nc2c1ncn2[C@@H]1O[C@H](CO[C@](Cc2ccc(-c3ccccc3OC(F)(F)F)cc2)(C(=O)O)c2cscn2)[C@@H](O)[C@H]1O] |
| 44 | 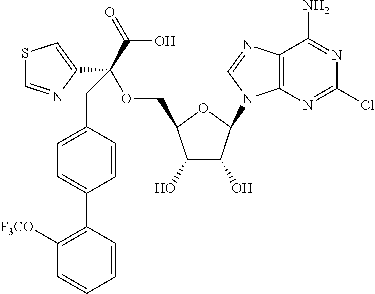 [SMILES: Nc1nc(Cl)nc2c1ncn2[C@@H]1O[C@H](CO[C@@](Cc2ccc(-c3ccccc3OC(F)(F)F)cc2)(C(=O)O)c2cscn2)[C@@H](O)[C@H]1O] |
| 45 | 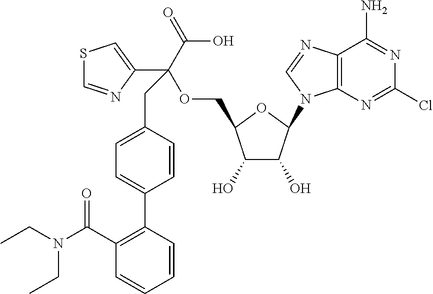 [SMILES: CCN(CC)C(=O)c1ccccc1-c1ccc(CC(OC[C@H]2O[C@@H](n3cnc4c(N)nc(Cl)nc43)[C@H](O)[C@@H]2O)(C(=O)O)c2cscn2)cc1] |
| 46 | 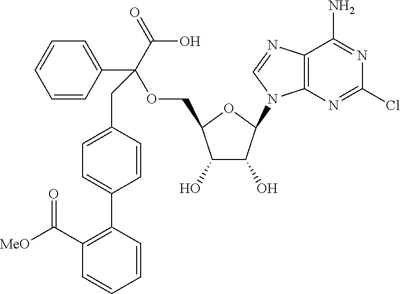 [SMILES: COC(=O)c1ccccc1-c1ccc(CC(OC[C@H]2O[C@@H](n3cnc4c(N)nc(Cl)nc43)[C@H](O)[C@@H]2O)(C(=O)O)c2ccccc2)cc1] |
| 47 | 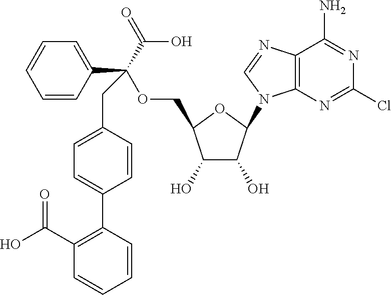 [SMILES: Nc1nc(Cl)nc2c1ncn2[C@@H]1O[C@H](CO[C@](Cc2ccc(-c3ccccc3C(=O)O)cc2)(C(=O)O)c2ccccc2)[C@@H](O)[C@H]1O] |
| 48 | 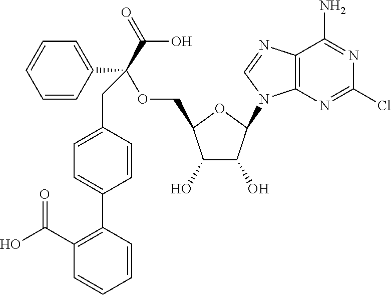 [SMILES: Nc1nc(Cl)nc2c1ncn2[C@@H]1O[C@H](CO[C@@](Cc2ccc(-c3ccccc3C(=O)O)cc2)(C(=O)O)c2ccccc2)[C@@H](O)[C@H]1O] |
| 49 | 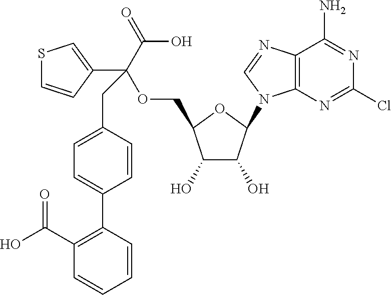 [SMILES: Nc1nc(Cl)nc2c1ncn2[C@@H]1O[C@H](COC(Cc2ccc(-c3ccccc3C(=O)O)cc2)(C(=O)O)c2ccsc2)[C@@H](O)[C@H]1O] |
| 50 | 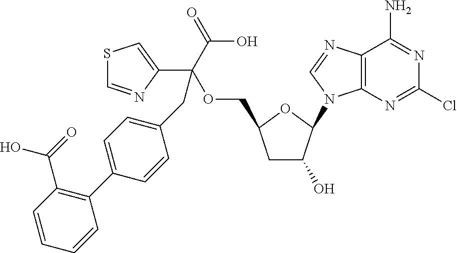 [SMILES: Nc1nc(Cl)nc2c1ncn2[C@@H]1O[C@H](COC(Cc2ccc(-c3ccccc3C(=O)O)cc2)(C(=O)O)c2cscn2)C[C@H]1O] |

or a pharmaceutically acceptable salt thereof.

33. A pharmaceutical composition comprising a compound according to any one of claims 1-32, or a pharmaceutically acceptable salt thereof, and one or more pharmaceutically acceptable excipients.

34. A method of inhibiting CD73 in a cell, comprising contacting the cell with a compound according to any one of claims 1-32, or a pharmaceutically acceptable salt thereof.

35. A method of treating a disease or disorder selected from cancer, cerebral and cardiac ischemic diseases, fibrosis, immune and inflammatory disorders, inflammatory gut motility disorder, neurological, neurodegenerative and CNS disorders and diseases, depression, Parkinson's disease, and sleep disorders, comprising administering a compound according to any one of claims 1-32, or a pharmaceutically acceptable salt thereof.

36. The method of claim 35, wherein the cancer is selected from bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head & neck cancer, Kaposi's sarcoma, kidney cancer, leukemia, liver cancer, lung cancer, lymphoma, melanoma, myeloma, ovarian cancer, pancreatic cancer, penile cancer, prostate cancer, testicular germcell cancer, thymoma and thymic carcinoma.

37. The method of claim 35, wherein the cancer is selected from breast cancer, brain cancer, colon cancer, fibrosarcoma, kidney cancer, lung cancer, melanoma, ovarian cancer, and prostate cancer.

38. The method of any one of claims 35-37, wherein the cancer is breast cancer.

39. The method of any one of claims 35-38, further comprising conjointly administering one or more additional chemotherapeutic agents.

40. The method of claim 39, wherein the one or more additional chemotherapeutic agents are selected from 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, I-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, ABT-263, afatinib dimaleate, axitinib, aminoglutethimide, amsacrine, anastrozole, APCP, asparaginase, AZD5363, Bacillus Calmette-Guérin vaccine (beg), bicalutamide, bleomycin, bortezomib, β-methylene-ADP (AOPCP), buserelin, busulfan, cabazitaxel, cabozantinib, campothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, N-(4-sulfamoylphenylcarbamothioyl) pivalamide, NF279, NF449, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, PPADS, procarbazine, quercetin, raltitrexed, ramucirumab, reactive blue 2, rituximab, rolofylline, romidepsin, rucaparib, selumetinib, sirolimus, sodium 2,4-dinitrobenzenesulfonate, sorafenib, streptozocin, sunitinib, suramin, talazoparib, tamoxifen, temozolomide, temsirolimus, teniposide, testosterone, thalidomide, thioguanine, thiotepa, titanocene dichloride, tonapofylline, topotecan, trametinib, trastuzumab, tretinoin, veliparib, vinblastine, vincristine, vindesine, vinorelbine, and vorinostat (SAHA).

41. The method of claim 39, wherein the one or more additional chemotherapeutic agents are selected from 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, I-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, APCP, β-methylene-ADP (AOPCP), capecitabine, cladribine, cytarabine, fludarabine, doxorubicin, gemcitabine, N-(4-sulfamoylphenylcarbamothioyl) pivalamide, NF279, NF449, PPADS, quercetin, reactive blue 2, rolofylline sodium 2,4-dinitrobenzenesulfonate, sumarin, and tonapofylline.

42. The method of claim 39, wherein the additional chemotherapeutic agent is an immuno-oncology agent.
